(19) United States
(12) Patent Application Publication
(10) Pub. No.: US 2011/0030074 A1
(43) Pub. Date: Feb. 3, 2011

(54) COMPOSITIONS AND METHODS FOR CANCER GENE DISCOVERY

(75) Inventors: Ronald A. Depinho, Brookline, MA (US); Lynda Chin, Brookline, MA (US); Richard Maser, Brookline, MA (US)

Correspondence Address: ROPES & GRAY LLP, PATENT DOCKETING 39/361, 1211 AVENUE OF THE AMERICAS, NEW YORK, NY, 10036-8704, US

(73) Assignee: DANA FARBER CANCER INSTITUTE, INC.

(21) Appl. No.: 12/601,052

(22) PCT Filed: May 21, 2008
(86) PCT No.: PCT/US08/06583

§ 371 Date: Jun. 11, 2010

Related U.S. Application Data
(60) Provisional application No. 60/931,294, filed on May 21, 2007.

(51) Int. Cl. A01K 67/027; C12Q 1/68; C12Q 1/02; A61K 39/395; A61P 35/00

(52) U.S. Cl. 800/10; 435/6; 435/29; 424/174.1

(57) ABSTRACT

The present invention features transgenic non-human mammalian animals being genetically modified to develop cancer. The invention also relates to methods for identifying genes or genetic elements that are potentially related to human cancers using an chromosomally unstable animal model. Information on such genetic alterations can be used to predict cancer therapeutic outcomes and to stratify patient populations to maximize therapeutic efficacy.

### CROSS REFERENCE TO RELATED APPLICATIONS

This application claims the benefit of and priority to U.S. Provisional Application No. 60/931,294, filed on May 21, 2007, the contents of which is hereby incorporated by reference in its entirety.

### GOVERNMENT SUPPORT

The work described herein was funded, in whole or in part, by Grant Number CA84628 (RO1) and CA84313 (UO1). The United States government may have certain rights in the invention.

### FIELD OF THE INVENTION

The present invention relates generally to the use of a genome unstable animal cancer model for cancer gene discovery.

### BACKGROUND INFORMATION

Cancer is a genetic disease driven by the stochastic acquisition of mutations and shaped by natural selection. Genomic instability, a hallmark of many human cancers, propagates these mutations, allowing cells to overcome critical barriers to unregulated growth, and may therefore herald a defining event in malignant transformation. Genomic instability is manifested by chromosomal aberrations, such as translocations and amplifications. How and when during the course of tumor progression significant genomic instability arises, and whether a cancer can be cured or even contained after that point, represent pivotal and largely unanswered questions.

Animal models for human carcinomas are valuable tools for the investigation and development of cancer therapies. Murine models having oncogenes incorporated into its genome, or tumor suppressor genes suppressed have been widely used for human cancer research. However, an impediment towards maximal utilization of murine models for guiding human cancer gene discovery efforts is the relatively benign cytogenetic profiles of most standard genetically engineered mouse models of cancer (see, e.g., N. Bardeesy, et al., Proc Natl Acad Sci USA 103 (15), 5947 (2006); M. Kim, et al., Cell 125 (7), 1269 (2006); L. Zender, et al., Cell 125 (7), 1253 (2006); A. Sweet-Cordero, et al., Genes Chromosomes Cancer 45 (4), 338 (2006)). These models do not reflect the global chromosomal aberrations associated with many types of human cancers.

Several cancer-prone murine models have recently been developed that more closely simulate the rampant chromosomal instability of human cancers. For example, Artandi et al. describe the development of epithelial cancers in a telomerase-definition p53-mutant mouse model (Nature 406 (6796), 641 (2000)); Zhu et. al describe oncogene translocation and amplification in a mouse model that is deficient in both p53 and nonhomologous end-joining (NHEJ) (Cell 109 (7), 811 (2002)); Olive et. al describe a Li-Fraumeni Syndrome mouse model having dominant p53 mutant alleles (Cell 119 (6), 847 (2004)); Lang et. al describe a Li-Fraumeni Syndrome mouse model having p53 missense mutations (Cell 119 (6), 861 (2004)); and Hingorani et. al describe a mouse model of pancreatic ductal adenocarcinoma, expressing mutant forms of TP53 and KRAS2 (Cancer Cell 7 (5), 469 (2005)). However, the frequency of chromosomal aberrations in these mouse models are relatively low, and the transgenic mice do not necessarily develop malignant cancer. To facilitate oncogenomic anlayses, there is a need to create new mammal models that are genetically modified to develop cancer, having chromosomal aberrations at a frequency that is comparable to human cancers.

### SUMMARY OF THE INVENTION

Highly rearranged and mutated cancer genomes present major challenges in the identification of pathogenetic events driving the cancer process. Here, we engineered lymphoma-prone mice with chromosomal instability to assess the utility of animal models in cancer gene discovery and the extent of cross-species overlap in cancer-associated copy number alterations. Integrating with targeted re-sequencing, our comparative oncogenomic studies identified FBXW7 and PTEN as commonly deleted or mutated tumor suppressors in human T-cell acute lymphoblastic leukemia/lymphoma (T-ALL). More generally, the murine cancers acquire widespread recurrent clonal amplifications and deletions targeting loci syntenic to alterations present in not only human T-ALL but also diverse tumors of hematopoietic, mesenchymal and epithelial types. These results thus support the view that murine and human tumors experience common biological processes driven by orthologous genetic events as they evolve towards a malignant phenotype. The highly concordant nature of genomic events encourages the use of genome unstable animal cancer models in the discovery of biologically relevant driver events in human cancer.

In one aspect, the invention provides a non-human transgenic mammal that is genetically modified to develop cancer, such that the genome of a cancer cell from the mammal comprises chromosomal structural aberrations at a frequency that is at least 5-fold higher than the frequency of chromosomal structural aberrations in such mammal without the genetic modification. In certain embodiments, the mammal is a rodent. In certain embodiments, the mammal is a mouse.

In certain embodiments, the mammal comprises engineered inactivation of: at least one allele of one or more genes encoding a protein involved in DNA repair function (such as a protein involved in non-homologous end joining (NHEJ), a protein involved in homologous recombination, or a DNA repair helicase), and at least one allele of one or more genes encoding a component that synthesizes and maintains telomere length. Alternatively, the mammal may comprise engineered inactivation of: at least one allele of one or more genes encoding a protein involved in DNA repair function and at least one allele of one or more genes encoding a DNA damage checkpoint protein. Alternatively, the mammal may comprise engineered inactivation of: at least one allele of one or more genes encoding a DNA damage checkpoint protein and at least one allele of one or more genes encoding a component that synthesizes and maintains telomere length.

In certain embodiments, the genome of the mammal further comprises at least one additional cancer-promoting modification, such as an activated oncogene, an inactivated tumor suppressor gene, or both.

In another aspect, the invention provides a method of identifying a chromosomal region of interest for the identification of a gene or genetic element that is potentially related to human cancer, comprising the step of: identifying a DNA copy number alteration in a population of cancer cells from a non-human mammal that is engineered to produce chromosomal instability. The chromosomal region of the DNA copy number alteration is a chromosomal region of interest for identifying a gene or genetic element that is potentially related to human cancer.

In certain embodiments, the DNA copy number alteration is recurrent in two or more cancer cells from the non-human mammal. The DNA copy number alteration can be a DNA gain or a DNA loss.

In another aspect, the invention provides a method of identifying a chromosomal region of interest for the identification of a gene or genetic element that is potentially related to human cancer, comprising the step of: identifying a chromosomal structural aberration in a population of cancer cells from a non-human mammal that is engineered to produce genome instability. A chromosomal region containing the chromosomal structural aberration is a chromosomal region of interest for identifying a gene or genetic element that is potentially related to human cancer.

In certain embodiments, the method further comprises the steps of: (1) identifying a DNA copy number alteration in the population of cancer cells from the non-human mammal, and (2) identifying a chromosomal region in the genome of the cancer cell of the non-human mammal that contains a chromosomal structural aberration and a DNA copy number alteration. The chromosomal region containing a chromosomal structural aberration and a DNA copy number alteration is a chromosomal region of interest for identifying a gene and genetic element that is potentially related to human cancer. In certain embodiments, the method further comprises the step of determining the uniform copy number segment boundary of the DNA copy number alteration.

In another aspect, the invention provides a method for identifying a potential human cancer-related gene, comprising the steps of: (a) identifying a chromosomal region of interest (e.g., comprising a gene or genetic element that is potentially related to human cancer); (b) identifying a gene or genetic element within the chromosomal region of interest in the non-human mammal, and (c) identifying a human gene or genetic element that corresponds to the gene or genetic element identified in step (b). The human gene or genetic element is a potential human cancer-related gene or genetic element. In certain embodiments, the human gene is orthologous, paralogous, or homologous to the gene or genetic element identified in step (b). In certain embodiments, the method further comprises the step of detecting a mutation in the non-human mammalian gene or genetic element identified in step (b), the human gene or genetic element identified in step (c), or both.

In another aspect, the invention provides a method of identifying a potential human cancer-related gene or genetic element, comprising the steps of: (a) detecting a DNA copy number alteration in a population of cancer cells from a non-human mammal that is engineered to produce genome instability, (b) identifying a gene or genetic element located within the boundaries of the DNA copy number alteration detected in step (a), and (c) identifying a human gene or genetic element that corresponds to the gene or genetic element identified in step (b) and that is located within the boundaries of a DNA copy number alteration or of a chromosomal structural aberration in a human cancer cell. The human gene or genetic element identified in step (c) is a gene or genetic element potentially related to human cancer.

In another aspect, the invention provides a method of identifying a potential human cancer-related gene or genetic element, comprising the steps of (a) detecting a chromosomal structural aberration in a population of cancer cells from a non-human mammal that is engineered to produce genome instability, (b) identifying a gene or genetic element located at the site of the chromosomal structural aberration detected in step (a), and (c) identifying a human gene or genetic element that corresponds to the gene or genetic element identified in step (b) and that is located within the boundaries of a DNA copy number alteration or at the site of a chromosomal structural aberration in a human cancer cell. The human gene or genetic element identified in step (c) is a gene or genetic element potentially related to human cancer. In certain embodiments, the method further comprises the step of detecting a mutation in the non-human mammalian gene or genetic element identified in step (b), the human gene or genetic element identified in step (c), or both.

In certain embodiments, the method further comprises the step of defining the minimum common region (MCR) of a recurrent gene copy number alteration. In certain embodiments, the MCR is defined by boundaries of overlap between two or more samples. In certain embodiments, the MCR is defined by the boundaries of a single tumor against a background of larger alteration in at least one other tumor.

In another aspect, the invention provides a method for identifying subjects with T-cell acute lymphoblastic leukemia (T-ALL) who may have a decreased response to γ-secretase inhibitor therapy, comprising detecting the expression or activity of FBXW7 in a tumor cell from the subject. A decreased expression or activity of FBXW7, as compared to a control, is indicative that the subject may have a decreased response to γ-secretase inhibitor therapy.

In certain embodiments, the method further comprises detecting the expression or activity of NOTCH1 in a tumor cell from the subject. An increased expression or activity of NOTCH1, as compared to a control, is indicative that the subject may have a decreased response to γ-secretase inhibitor therapy.

In another aspect, the invention provides a method for identifying subjects with T-ALL that may benefit from treatment with a PI3K pathway inhibitor, comprising detecting the expression or activity of PTEN in a tumor cell from the subject. A decreased expression or activity of PTEN, as compared to a control, is indicative that the subject may benefit from a treatment with a PI3K inhibitor. In certain embodiments, the method further comprises treating the subject with a PI3K inhibitor.

In another aspect, the invention provides a method of assessing whether a subject is afflicted with cancer or at risk for developing cancer, comprising: determining the expression or activity level of at least one cancer gene or candidate cancer gene located in an amplified MCR in Table 1 in a biological sample from the subject. An increase in the expression or activity the gene, as compared to a control, indicates that the subject is afflicted with cancer or at risk for developing cancer. Alternatively, if there is a decrease in the expression or activity of a cancer gene or candidate cancer gene located in a deleted MCR in Table 1, as compared to a control, the decreased expression or activity level also indicates that the subject is afflicted with cancer or at risk for developing cancer.

In another aspect, the invention provides a method of assessing whether a subject is afflicted with cancer or at risk for developing cancer, the method comprising: determining the copy number of at least one amplified minimal common region (MCR) listed in Table 1 in a biological sample from the subject. An increased copy number of the MCR in the sample, as compared to the normal copy number of the MCR, indicates that the subject is afflicted with cancer or at risk for developing cancer. Alternatively, a decreased copy number of a deleted MCR (also listed in Table 1) in the sample, as compared to the normal copy number of the MCR, also indicates that the subject is afflicted with cancer or at risk for developing cancer. The normal copy number of an MCR is typically one per chromosome.

In another aspect, the invention provides a method for monitoring the progression of cancer in a subject, the method comprising: a) determining in a biological sample from the subject at a first point in time, the expression or activity level of a cancer gene or a candidate cancer gene listed in Table 1; b) repeating step a) at a subsequent point in time; and c) comparing the expression or activity of the gene in steps a) and b), and therefrom monitoring the progression of cancer in the subject.

In another aspect, the invention provides a method of assessing the efficacy of a test agent for treating a cancer in a subject, comprising: a) determining the expression or activity level of at least one cancer gene or a candidate cancer gene located in an amplified MCR in Table 1 in a biological sample from the subject in the presence of the test agent; and b) determining the expression or activity level of the gene in a biological sample from the subject in the absence of the test agent. A decreased expression or activity of the gene in step (a), as compared to that of (b), is indicative of the test agent's potential efficacy for treating the cancer in the subject. Alternatively, if the test agent increases the expression or activity of at least one cancer gene or a candidate cancer gene located in a deleted MCR in Table 1, the test agent is also potentially effective for treating the cancer in a subject.

In another aspect, the invention provides a method of assessing the efficacy of a therapy for treating cancer in a subject, the method comprising: a) determining the expression or activity level of at least one cancer gene or a candidate cancer gene located in an amplified MCR in Table 1 in a biological sample from the subject prior to providing at least a portion of the therapy to the subject; and b) determining the expression or activity level of the gene in a biological sample from the subject following provision of the portion of the therapy. A decreased expression or activity of the gene in step (a), as compared to that of (b), is indicative of the therapy's efficacy for treating the cancer in the subject. Alternatively, if the therapy increases the expression or activity of at least one cancer gene or a candidate cancer gene located in a deleted MCR in Table 1, the therapy is also potentially effective for treating the cancer in a subject.

In another aspect, the invention provides a method of treating a subject afflicted with cancer comprising administering to the subject an agent that decreases the expression or activity level of at least one cancer gene or candidate cancer gene located in am amplified MCR in Table 1. Alternatively, the invention provides a method of treating a subject afflicted with cancer comprising administering to the subject an agent that increases the expression or activity level of at least one cancer gene or candidate cancer gene located in a deleted MCR in Table 1.

In certain embodiments, the agent is an antibody, or its antigen-binding fragment thereof, that specifically binds to a cancer gene or candidate cancer gene listed in Table 1.

In another aspect, the invention provides a method of assessing whether a subject is afflicted with cancer or at risk for developing cancer, the method comprising: determining the copy number of at least one minimal common region (MCR) listed in Table 5 in a biological sample from the subject. A change of copy number of the MCR in the sample, as compared to the normal copy number of the MCR, indicates that the subject is afflicted with cancer or at risk for developing cancer. The normal copy number of an MCR is typically one per chromosome.

In certain embodiments, the cancer is lymphoma. In certain embodiments, the lymphoma is T-ALL.

In another aspect, the invention provides a method of assessing whether a subject is afflicted with cancer or at risk for developing cancer, by comparing the copy number of an MCR, identified using a genome-unstable non-human mammal model (including a genome-unstable mouse model of the invention), with the normal copy number of the MCR. The normal copy number of an MCR is typically one per chromosome.

### BRIEF DESCRIPTION OF THE DRAWINGS

FIG. 1: Spectral Karyotype (SKY) profiles of TKO tumors. G-band and SKY images of representative metaphases for selected TKO tumors with and without telomere dysfunction. FIG. 1A represents G0 (mTerc +/+ or +/−) and FIG. 1B represents G1-G4 (mTerc−/−) TKO tumors. The pictures show an overall increase in frequency of chromosome structural aberrations in TKO tumors with telomere dysfunction. Nonreciprocal translocations and chromosomal fragments are marked by arrows. FIG. 1C shows representative array-CGH Log 2 ratio plots of syntenic murine TKO (left; A689) and human (right; HPB-ALL) TCRB deletions. Y axis, log 2 ratio of copy number (normal set at log 2=0); amplifications are above and deletions are below this axis; X axis, chromosome position.

FIG. 2. Characterization of the TKO model. FIG. 2A is a graph showing Kaplan-Meier curve of thymic lymphoma-free survival for G3-G4 TKO mice on p53 wildtype, heterozygous and null background. FIG. 2B shows the loss of heterozygosity for p53 using PCR; N, normal; T, tumor. FIG. 2C is a representative FACS profile of TKO tumor, using antibodies against cell surface markers CD4 and CD8. FIG. 2D is a representative SKY images from metaphase spreads from G0 (top) and G1-G4 (bottom) thymic lymphomas. Of equal number of metaphase spreads (90), 410 aberrations per 4533 chromosomes (9%) were found among G0 versus 1257 per 3659 (34%) among G1-G4 TKO tumors. No significant differences in ploidy level were observed. FIG. 2E is a plot showing quantification of total number of cytogenetic aberrations detected by SKY in G0 (blue) and G1-G4 (red) thymic lymphomas. Darker color indicates proportion of events representing non-reciprocal translocations and lighter color indicates proportion representing dicentric/Robertsonian-like rearrangements. FIG. 2F is a recurrence plot of CNAs defined by array-CGH for 35 TKO lymphomas. X axis represents physical location of each chromosomes, and Y axis represents % of tumors exhibiting copy number alterations. The percentage of tumors harboring gains, amplifications, losses and deletions for each locus is depicted according to the following scheme: dark red (gains with a log 2 ratio=>0.3) and green (loss with a log 2 ratio<=−0.3) are plotted along with bright red (Amplifications with a log₂ ratio=>0.6) and bright green (deletions with log₂ ratio<=−0.6). Location of physiologically-relevant CNAs at Tcrβ, Tcrα/δ, and Tcrγ is indicated with arrows, and other loci discussed in the text (Notch1, Pten) are indicated by asterisks.

FIG. 3: Notch1 array-CGH and SKY. FIG. 3A shows a representative array-CGH Log 2 ratio plot from murine TKO lymphoma A1052 showing focal amplification targeting the 3′-end of Notch1 and its location relative to other genes in the region (http://genome.ucsc.edu/), NBCI mouse build 34. Y axis, log 2 ratio of copy number (normal set at log 2=0); amplifications are above and deletions are below this axis; X axis, chromosome position. FIG. 3B are SKY analyses of murine TKO tumors A1052 and A895 cells that harbor chromosome 2 amplifications which target the 3′ end of Notch1. Upper panels: metaphase spreads from the indicated tumors showing non-reciprocal translocations involving murine chromosome 2, marked by arrows; the asterisk indicates an abnormal band chr2A3. Lower panels: representative SKY images of individual rearranged chromosomes involving chromosome 2 and other chromosomes, as indicated. Each panel is a composite of raw spectral image (left), DAPI image (middle), and computer-interpreted spectral image (right) for the indicated rearranged chromosome. FIG. 3C shows breakpoint separating two contiguous BAC probes overlapping at Notch1, using FISH. Red signal, BAC probe RP24-369L23; green signal, BAC probe RP23-412O13.

FIG. 4. NOTCH1 alterations in both murine and human T-ALLs. FIG. 4A is a graphic illustration of Location of sequence alterations affecting Notch1 in murine TKO and human T-ALL tumors. Each marker is indicative of an individual cell line/patient. FIG. 4B shows Western blotting analysis of murine full-length Notch1 (FL; top), cleaved active Notch1 (V1744; middle), and tubulin loading control (bottom). High levels of activated Notch1 protein were expressed in many TKO tumors, including those harboring 3′ translocations (in blue: A577, A1052, A1252) and truncating deletion mutations (in red: A494, A1040), in which faster migrating V1744 forms are apparent. Human ALL-SIL (left) and normal mouse thymus (right) samples were loaded for controls. FIG. 4C shows that high levels of Notch1 mRNA correlate with high mRNA levels of known downstream targets of Notch1 protein, as assessed by expression profiling of TKO tumors. Each bar represents an individual probe set. Samples in blue lettering harbor 3′ translocations near Notch1; samples in red lettering harbor truncating deletion mutations, as indicated for FIG. 4B.

FIG. 5. FBXW7 alterations are common in human T-ALL and conserved in the murine TKO tumors. FIG. 5A are a group of Log 2 ratio array-CGH plots showing conservation of CNAs resulting in deletion of FBXW7 in both mouse TKO and human T-ALL cell lines; the genomic location of Fbxw7 is indicated in green. Y axis, log 2 ratio of copy number (normal set at log 2=0); amplifications are above and deletions are below this axis; X axis, chromosome position. FIG. 5B shows relative expression level of mouse Fbxw7 mRNA, as assessed by real-time qPCR in the indicated murine TKO tumors. FIG. 5C is a graphic illustration of location of mutations in human FBXW7 identified in a panel of human T-ALL patients and cell lines. Each marker represents an individual cell line/patient.

FIG. 6: Focal deletion of Pten in TKO tumors. FIG. 6A is a representative array-CGH Log 2 ratio plot from a TKO lymphoma showing focal deletion encompassing Pten, and its location relative to other genes in the region (http://genome.ucsc.edu/, NBCI mouse build 34). Y axis, log 2 ratio of copy number (normal set at log 2=0); amplifications are above and deletions are below this axis; X axis, chromosome position. FIG. 6B summarizes the result of real-time qPCR (showing deletion in several tumors), with a graphic illustration of real-time qPCR with primer sets to the indicated regions (arrows) and the location of array-CGH 60-mer oligo probes (Agilent 44K array). A494 is shown as a control without evidence of deletion.

FIG. 7. Conservation of PTEN genetic alterations in human and mouse T-ALLs. FIG. 7A are a group of Log 2 ratio array-CGH plots demonstrating conservation of CNAs resulting in deletion of PTEN in both mouse TKO and human T-ALL cell lines; the genomic location of Pten is indicated in green. Y axis, log 2 ratio of copy number (normal set at log 2=0); amplifications are above and deletions are below this axis; X axis, chromosome position. FIG. 7B is a Western blotting analysis, showing the expression level of PTEN, phospho-Akt, and Akt in a panel of murine TKO and human T-ALL cell lines. BE13 and PEER are synonymous lines. Tubulin was probed simultaneously as a loading control. Samples in red harbor confirmed sequence mutations; samples in blue harbor aCGH-detected deletions. FIG. 7C are a group of Log 2 ratio array-CGH plots showing the effects of CNAs on other members of the Pten-Akt axis in murine TKO tumors. The location of each gene (Akt1, Tsc1) is shown in green.

FIG. 8: TKO cells with Pten mutation/deletion are sensitive to inhibition of phospho-Akt by the drug triciribine. Cells were plated in triplicate and exposed to the indicated doses of triciribine or vehicle alone for 48 hours and then quantified by MTS assay for viable cells. The fraction of surviving cells is plotted relative to survival in vehicle alone (set at 1). Tumor A1040 retains wildtype Pten expression and A1005 harbors a point mutation in one copy of Pten, whereas cell lines A577, A1240, A1252, and A494 are deficient for Pten expression.

FIG. 9. Substantial overlap between genomic alterations of murine TKO lymphomas and human tumors of diverse origins. FIG. 9A summarizes the result of statistical analysis of the cross-species overlap. We obtained Human array-CGH profiles from the indicated tumor types. We further defined MCRs as described in the Examples section (in particular, Example 4). Characteristics of each set are listed on the left portion of the panel. The number of TKO MCRs (amp, amplifications; del, deletions) with syntenic overlap with corresponding human CGH dataset is indicated on the right side of the panel, with p value for each based on 10,000 permutations. FIG. 9B are a group of Pie-chart representation of numbers of TKO MCRs (indicated within each segment) with syntenic overlap identified in one or multiple human tumor types (indicated by different colors of the segments); left, amplifications; right, deletions. For example, 21 of the 61 syntenic amplifications in FIG. 9A were observed in 2 different human tumor CGH datasets. FIG. 9C are a group of Venn diagram representation of the degree of overlap between murine TKO MCRs and MCRs from human cancers of T-ALL, multiple myeloma, or solid tumors (encompassing glioblastoma, melanoma, and pancreatic, lung, and colon adenocarcinoma).

### DETAILED DESCRIPTION OF THE INVENTION

In vivo cancer models used for the discovery of cancer-related genes and therapeutic cancer targets typically produce cancer cells with benign chromosomal profiles, i.e., nearly normal chromosomal stability. In contrast, in naturally occurring human cancer, cancer cell genomes display widespread instability as evidenced by chromosomal structural aberrations. Accordingly, the present invention provides an in vivo cancer model with a destabilized genome (“genome unstable”).

The genomes of cancer cells from the genome unstable model of the invention simulate the chromosomal instability displayed by human cancer cell genomes The genome unstable cancer model of the invention, thus, provides significant advantages for the discovery of genes and genetic elements involved in human cancer initiation, maintenance and progression. The chromosomal aberrations in cancer cells from the model, particularly recurrent aberrations, permit investigation of chromosomal events in cancer that is not possible in cancer models with “benign” chromosomal profiles. Such chromosomal aberrations also focus attention on particular regions of the genome more likely to harbor cancer-related elements. The validation herein of a genome unstable mouse cancer model that generates chromosomal and genetic events that mirror those in multiple types of human cancers provides an important new tool for the discovery of cancer-related genes and therapeutic targets of relevance to human cancer. Although useful by itself to discover genes and genetic elements relevant to human cancer, the genome unstable model of the invention also can be used as a background for establishing other cancer models, including known cancer models. Layering genetic modifications in known oncogenes and/or tumor suppressors onto the genome unstable model of the invention provides improved models that more closely replicate naturally occurring cancer. Even more importantly, the genome unstable model of the invention permits cross-species comparison with human cancer genomes to identify shared chromosomal and genetic events. Such shared events provide a powerful guide for the discovery of cancer-related genes and therapeutic targets.

### 1. DEFINITIONS

Throughout this specification and embodiments, the word “comprise” or variations such as “comprises” or “comprising” will be understood to imply the inclusion of a stated integer or group of integers but not the exclusion of any other integer or group of integers.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, cell and cancer biology, virology, immunology, microbiology, genetics and protein and nucleic acid chemistry described herein are those well known and commonly used in the art.

### 2. ANIMAL MODELS

Most standard genetically engineered mouse models of cancer have relatively benign cytogenetic profiles. These genomically stable models do not reflect the widespread chromosomal instability that is typical of human genomes in cancer. It has been reported that in most “genome-stable” murine tumor models, about 20 to 40 chromosomal aberrations were detected per genome, or, less than 0.1 chromosomal rearrangements per chromosome.

Accordingly, in one aspect, the invention provides a non-human animal that is genetically modified to develop cancer, wherein the genomes of cancer cells from the animal display enhanced chromosomal instability as evidenced by a frequency of chromosomal structural aberration that approaches or matches that seen in human cancer cells. In various embodiments, the frequency of chromosomal structural aberrations in a population of cancer cells from the non-human animal model is at least 1.5-fold, 2-fold, 3-fold, 4-fold, 5-fold or 10-fold higher than the frequency of chromosomal structural aberrations in such mammal without the genetic modification, whether defined on a per-genome or per-chromosome basis.

The frequency of chromosomal abnormalities can be based on the average number of such abnormalities per genome or per chromosome, or the average number of a particular type of chromosomal abnormality per genome, or the average number of aberrations in a particular chromosome. Methods of measuring chromosomal alterations are known in the art (see, e.g., R. C. O'Hagan, et al., Cancer Res 63 (17), 5352 (2003); N. Bardeesy, et al., Proc Natl Acad Sci USA 103 (15), 5947 (2006); M. Kim, et al., Cell 125 (7), 1269 (2006); L. Zender, et al., Cell 125 (7), 1253 (2006)), and are further disclosed below. Cancer cells from the genome unstable non-human animal model of the invention will have an enhanced frequency of chromosomal aberrations compared to cells derived from comparable non-human animal models lacking the genome destabilizing mechanisms described above, by at least one of the aforementioned parameters.

A chromosomal structural aberration may be any chromosomal abnormality resulting from DNA gains or losses, DNA amplification, DNA deletion, and DNA translocation. Exemplary chromosomal structural aberrations include, for example, sister chromatid exchanges, multi-centric chromosomes, inversions, gains, losses, reciprocal and non-reciprocal translocations (NRTs), p-p robertsonian-like translocations of homologous and/or non-homologous chromosomes, p-q chromosome arm fusions, and q-q chromosome arm fusions.

The genetic modifications in the genome unstable animal model of the invention can be in any gene or genetic element that renders the animal cancer-prone and affects genome structure or genome stability, so that the modifications destabilize the genome, as evidenced by an increased frequency of chromosomal structural aberrations in the genomes and/or chromosomes of cancer that develops in the animal compared to genomes and/or chromosomes in comparable animal models lacking such genome destabilizing mechanisms. Genetic elements include [DNA that is not translated to produce a protein product such as micro RNA, expression control sequences including DNA transcription factor binding sites, RNA transcription initiation sites, promoters, enhancers, response elements and the like. In some embodiments the genetic modifications inactivate a gene or genetic element involved in chromosomal structural stability or integrity. Inactivation may be by directly inactivating the gene or genetic element, by suppressing the expression, or by inactivating or inhibiting the activity of a gene product, which can be a nucleic acid product including RNA or a protein gene product

In some embodiments, the genetic modifications comprise inactivation of at least one allele of one or more genes or genetic elements involved in DNA repair and inactivation of at least one allele of one or more genes or genetic elements involved in a DNA damage checkpoint. In some embodiments, the genetic modifications further comprise inactivation of at least one allele of a gene or genetic element involved in telomere maintenance. In any of the foregoing embodiments, both alleles of the DNA repair related, DNA damage checkpoint related and/or telomere maintenance related genes or genetic elements may be inactivated.

Any gene or genetic element involved in DNA repair or in a DNA damage checkpoint can be inactivated in the genome unstable model of the invention. Many such genes and genetic elements in humans an other mammals will be known to those of skill in the art. See, for example, R. D. Wood et al., Human DNA Repair Genes, Science, 291: 1284-1289 (February 2001); R A Bulman, S D Bouffler, R Cox and T A Dragani, Locations of DNA Damage Response and Repair Genes in the Mouse and Correlation with Cancer Risk Modifiers, National Radiological Protection Board Report, October 2004 (ISBN 0-85951-544-3). The mouse DNA repair gene database is available at the UK Health Protection Agency website.

They include, for example, genes encoding base excision repair (BER) proteins such as ung, smug1, mbd4, tdg, off1, myh, nth1, mpg, ape1, ape2, lig3, xrcc1, adprt, adprtl2 and adprtl3 or species homologs thereof; mismatch excision repair proteins such as msh2, msh3, msh4, msh5, msh6, pms1, pms3, mlh1, mlh3, pms2l3 and pms2l4 or species homologs thereof; nucleotide excision repair (NER) proteins, non-homologous end joining (NHEJ) proteins, homologous recombination proteins, DNA polymerases, editing and processing nucleases and DNA repair helicases, among others. Wood et al., supra.

Exemplary NHEJ proteins include Ligase4, XRCC4, H2AX, DNAPKcs, Ku70, Ku80, Artemis, Cernunnos/XLF, MRE11, NBS1, and RAD50. Exemplary homologous recombination proteins include RAD51, RAD52, RAD54, XRCC3, RAD51C, BRCA1, BRCA2 (FANCD1), FANCA, FANCB, FANCC, FANCD2; FANCE, FANCF, FANCG, FANCJ (BRIP1/BACH1), FANCL, and FANCM. Exemplary DNA repair helicases include BLM and WRN.

Any gene or genetic element involved in a DNA damage checkpoint can be used in the genome unstable model of the invention. Information about many such genes and genetic elements is readily available and will be well-known those of skill in the art. Exemplary DNA checkpoint proteins include sensor proteins such as RAD1, RAD9, RAD17, HUS1, MRE11, Rad50, and NBS1; mediators such as ATRIP; phosphoinositide 3-kinase related kinase (PIKK) family proteins such as ATM, ATR, SMG-1 and DNA-PK; checkpoint kinases such as Chk1 and Chk2; and effector proteins such as p53, p63, p73, CDC25A, B and C, p21 and 14-3-3β,γ,ξ,σ,ε,η,τ APC; BRCA1, MDM2, MDM4, NBS1, RAD24, RAD 25, RAD50, MDC1, SMC1, and claspin.

In one embodiment of the genome unstable model of the invention, the non-human transgenic animal further comprises engineered inaction of at least one allele of one or more genes or genetic elements involved in synthesizing or maintaining telomere length. In some embodiments, the non-human transgenic mammal is engineered for decreased telomerase activity, for example by inactivation of telomerase reverse transcriptase, Tert, or telomerase RNA (Terc). In some embodiments the genetic modification decreases the activity of a protein affecting telomere structure such as capping function. Exemplary proteins that affect telomere structure include TRF1, TRF2, POT1a, POT1b, RAP1, TIN2, and TPP1.

The non-human genome unstable model of the invention may be any animal, including, fish, birds, mammals, reptiles, amphibians. Preferably, the animal is a mammal, including rodents, primates, cats, dogs, goats, horses, sheep, pigs, cows. In preferred embodiments, the mammal is a mouse.

The genome unstable animal models of the invention include animals in which all or only some portion of cells comprise the genetic modifications that create genome instability. In some embodiments, the germ cells of the animal comprise the genetic modifications.

In some embodiments, the genome unstable model comprises inactivation of one or both alleles of atm, terc or p53 or any combination of those genes. In a particular embodiment, one or both alleles of all three genes are inactivated. In some embodiments both alleles of atm are inactivated. In a particular embodiment, both alleles of all three genes are inactivated.

Also within the invention are tissues and cells from the genome unstable model of the invention, including somatic cells, germ cells, stem cells including embryonic stem cells, differentiated cells and undifferentiated cells. The cells may be cancer cells, non-cancer cells, or pre-cancer cells.

Inactivation of a gene or a genetic element in the genome unstable animal model of the invention can be achieved by any means, many of which are well-known to those of skill in the art. Such means include deletion of all or part of the gene or genetic element or introducing an inactivating mutation (lesion) in the gene or genetic element. Deletion of all or a portion of a gene or genetic element may be by knock-out such as by homologous recombination or techniques using Cre recombinase (e.g., a Cre-Lox system). Deletions including knock-outs can be conditional knock-outs, where alteration of a nucleic acid sequences can occur upon, for example, exposure of the animal to a substance that promotes gene alteration, introduction of an enzyme that promotes recombination at the gene site (e.g., Cre in the Cre-lox system), or other method for directing the gene alteration. Conditional or constitutive knock-outs can be tissue-specific, temporally-specific (e.g., occurring during a particular developmental stage) or both.

Inactivating mutations may be introduced using any means, many of which are well known. Such methods include site directed mutagenesis for example using homologous recombination or PCR. Such mutations may be introduced in the 5′ untranslated region (UTR) of a gene, including in an expression control region, in a coding region (intron or exon) or in the 3′ UTR.

The expression or activity of a gene or genetic element also may be accomplished by any means including but not limited to RNA interference, antisense including triple helix formation and ribozymes including RNaseP, leadzymes, hairpin ribozymes and hammerhead ribozymes.

In some embodiments, the genome unstable animal model of the invention further comprises one or more additional cancer-promoting genetic modifications including but not limited to the introduction of one or more activated oncogenes, modifications to increase the expression of one or more oncogenes, targeted inactivation of one or more tumor-suppressors, or combinations of the foregoing. Such additional cancer-promoting modifications may be inducible, tissue specific, temporally specific or any combination of the three. For example, an oncogene can be introduced into the genome using an expression cassette that includes in the 5′-3′ direction of transcription, a transcriptional and translational initiation region that is associated with gene expression in a specific tissue type, an oncogene, and a transcriptional and translational termination region functional in the host animal. One or more introns may also be present. In addition to the oncogene of interest, a detectable marker, such as GFP (and its variants), luciferase, and lacZ may be optionally operably linked to the oncogene and co-expressed. Similarly, a tumor-suppressor-gene may be inactivated using, for example, gene targeting technology.

Introducing additional cancer-promoting modifications into a genome-unstable animal model described herein creates a powerful tool for cancer gene discovery. For example, Kras activation and p53 mutation in pancreas are known to cause pancreas cancer in human. A genome-unstable model having pancreas-specific Kras activation, p53 inactivation (and optionally, a decreased telomere function) would greatly facilitate the discovery of pancreas cancer gene in human.

The cancer in the genome unstable model any type of cancer, including carcinoma, sarcoma, myeloma, leukemia, lymphoma or mixed cancer types. The cancer can arise from any tissue type including epithelial tissue, mesenchymal tissue, nervous tissue and hematopoietic tissue and be located in any organ or tissue of the body. The frequency of chromosomal aberrations can be determined in cells from any of the aforementioned cancers and can be from a primary tumor, a secondary tumor, a metastatic tumor, a tumor recurrence perhaps normal cells derived from said genomically unstable model that were genetically manipulated in vitro, through additional oncogene activation and tumor suppressor gene inactivation introduced by those knowledgeable in the art, to become cancerous

The genome unstable mouse model of the invention may develop any cancer including but not limited to acral lentiginous melanoma, actinic keratoses, adenocarcinoma, adenoid cystic carcinoma, adenomas, adenosarcoma, adenosquamous carcinoma, adrenocortical carcinoma, AIDS-related lymphoma, anal cancer, anaplastic glioma, astrocytic tumors, astrocytomas, bartholin gland carcinoma, basal cell carcinoma, biliary tract cancer, bone cancer, bile duct cancer, bladder cancer, brain stem glioma, brain tumors, breast cancer, bronchial gland carcinomas, capillary carcinoma, carcinoids, carcinoma, carcinosarcoma, cavernous, central nervous system lymphoma, cerebral astrocytoma, cervical cancer, connective tissue cancer, cholangiocarcinoma, chondosarcoma, choroid plexus papilloma/carcinoma, clear cell carcinoma, colon cancer, colorectal cancer, cutaneous T-cell lymphoma, cystadenoma, endodermal sinus tumor, endometrial hyperplasia, endometrial stromal sarcoma, endometrioid adenocarcinoma, ependymal, ependymoma, epitheloid, esophageal cancer, Ewing's sarcoma, extragonadal germ cell tumor, eye cancer, fibrolamellar, focal nodular hyperplasia, gallbladder cancer, gangliogliomas, gastric cancer, gastrinoma, germ cell tumors, gestational trophoblastic tumor, glioblastoma multiforme, glioma, glucagonoma, head and neck cancer, hemangiblastomas, hemangioendothelioma, hemangiomas, hepatic adenoma, hepatic adenomatosis, hepatocellular carcinoma, Hodgkin's lymphoma, hypopharyngeal cancer, hypothalamic and visual pathway glioma, childhood, insulinoma, intaepithelial neoplasia, interepithelial squamous cell neoplasia, intraocular melanoma, intra-epithelial neoplasm, invasive squamous cell carcinoma, large cell carcinoma, islet cell carcinoma, Kaposi's sarcoma, kidney cancer, laryngeal cancer, leiomyosarcoma, lentigo maligna melanomas, leukemia-related disorders, lip and oral cavity cancer, liver cancer, lung cancer, lymphoma, malignant mesothelial tumors, malignant thymoma, medulloblastoma, medulloepithelioma, melanoma, meningeal, merkel cell carcinoma, mesothelial, metastatic carcinoma, mucoepidermoid carcinoma, multiple myeloma/plasma cell neoplasm, mycosis fungoides, myelodysplastic syndrome, myeloproliferative disorders, nasal cavity and paranasal sinus cancer, nasopharyngeal cancer, neuroblastoma, neurofibromatosis, neuroepithelial adenocarcinoma nodular melanoma, non-Hodgkin's lymphoma, non-small cell lung cancer, oat cell carcinoma, oligodendroglial, oligoastrocytomas, oral cancer, oropharyngeal cancer, osteosarcoma, pancreatic polypeptide, ovarian cancer, ovarian germ cell tumor, pancreatic cancer, papillary serous adenocarcinoma, pineal cell, pituitary tumors, plasmacytoma, pseudosarcoma, pulmonary blastoma, parathyroid cancer, penile cancer, pheochromocytoma, pineal and supratentorial primitive neuroectodermal tumors, pituitary tumor, plasma cell neoplasm, pleuropulmonary blastoma, prostate cancer, rectal cancer, renal cell carcinoma, cancer of the respiratory system, retinoblastoma, rhabdomyosarcoma, sarcoma, serous carcinoma, skin cancer, small cell carcinoma, small intestine cancer, soft tissue carcinomas, somatostatin-secreting tumor, squamous carcinoma, squamous cell carcinoma, stomach cancer, stromal tumors, submesothelial, superficial spreading melanoma, supratentorial primitive neuroectodermal tumors, testicular cancer, thyroid cancer, undifferentiatied carcinoma, urethral cancer, uterine sarcoma, uveal melanoma, verrucous carcinoma, vaginal cancer, vipoma, vulvar cancer, Waldenstrom's macroglobulinemia, well differentiated carcinoma, and Wilm's tumor.

The animal models described herein are typically obtained using transgenic technologies. Transgenic technologies are well known in the art. For example, transgenic mouse can be prepared in a number of ways. A exemplary method for making the subject transgenic animals is by zygote injection. This method is described, for example in U.S. Pat. No. 4,736,866. The method involves injecting DNA into a fertilized egg, or zygote, and then allowing the egg to develop in a pseudo-pregnant mother. The zygote can be obtained using male and female animals of the same strain or from male and female animals of different strains. The transgenic animal that is born is called a founder, and it is bred to produce more animals with the same DNA insertion. In this method of making transgenic animals, the exogenous DNA typically randomly integrates into the genome by a non-homologous recombination event. One to many thousands of copies of the DNA may integrate at one site in the genome.

### 3. METHODS OF IDENTIFYING CANCER-RELATED GENES

In another aspect, the invention provides methods for identifying genes and genetic elements involved in cancer initiation, maintenance and/or progression in humans utilizing the genome unstable model of the invention. The gene discovery and identification methods are based on the surprising discovery described herein that chromosomal structural aberrations, copy number alterations and mutations in cancer cells in a genome unstable mouse model have syntenic counterparts (i.e., occurring in evolutionarily related chromosomal regions) in human cancer cells.

Accordingly, in one embodiment, the invention provides a method of identifying a chromosomal region of interest for the identification of a gene that is potentially related to human cancer, comprising the step of identifying a DNA copy number alteration in a population of cancer cells from a non-human, genome-unstable mammal described above. The chromosomal region where the DNA copy number alteration occurred is a chromosomal region of interest for the identification of a gene or genetic element (such as microRNAs) that is potentially related to human cancer.

A DNA copy number alteration may be a DNA gain (such as amplification of a genomic region) or a DNA loss (such as deletion of a genomic region). Methods of evaluating the copy number of a particular genomic region are well known in the art, and include, hybridization and amplification based assays. According to the methods of the invention, DNA copy number alterations may be identified using copy number profiling, such as comparative genomic hybridization (CGH) (including both dual channel hybridization profiling and single channel hybridization profiling (e.g. SNP-CGH)). Other suitable methods including fluorescent in situ hybridization (FISH), PCR, nucleic acid sequencing, and loss of heterozygosity (LOH) analysis may be used in accordance with the invention.

In one embodiment of the invention, the DNA copy number alterations in a genome are determined by copy number profiling.

In some embodiments of the invention, the DNA copy number alterations are identified using CGH. In comparative genomic hybridization methods, a “test” collection of nucleic acids (e.g. from a tumor or cancerous cells) is labeled with a first label, while a second collection (e.g. from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the gene copy number, corresponding to the specific probe used. A cytogenetic representation of DNA copy-number variation can be generated by CGH, which provides fluorescence ratios along the length of chromosomes from differentially labeled test and reference genomic DNAs.

In some embodiments of the present invention, the DNA copy number alterations are analyzed by microarray-based CGH (array-CGH). Microarray technology offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6, (1999), Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211 and others.

The DNA used to prepare the CGH arrays is not critical. For example, the arrays can include genomic DNA, e.g. overlapping clones that provide a high resolution scan of a portion of the genome containing the desired gene or of the gene itself. Genomic nucleic acids can be obtained from, e.g., HACs, MACs, YACs, BACs, PACs, PIs, cosmids, plasmids, inter-Alu PCR products of genomic clones, restriction digests of genomic clones, cDNA clones, amplification (e.g., PCR) products, and the like. Arrays can also be obtained using oligonucleotide synthesis technology. For example, see, e.g., light-directed combinatorial synthesis of high density oligonucleotide arrays U.S. Pat. No. 5,143,854 and PCT Patent Publication Nos. WO 90/15070 and WO 92/10092.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other suitable methods include are the nucleic acid sequence based amplification (NASBAO, Cangene, Mississauga, Ontario) and Q Beta Replicase systems.

In one embodiment of the invention, the DNA copy number alterations in a genome are determined by single channel profiling, such as single nucleotide polymorphism (SNP)-CGH. Traditional CGH data consists of two channel intensity data corresponding to the two alleles. The comparison of normalized intensities between a reference and subject sample is the foundation of traditional array-CGH. Single channel profiling (such as SNP-CGH) is different in that a combination of two genotyping parameters are analyzed: normalized intensity measurement and allelic ratio. Collectively, these parameters provide a more sensitive and precise profile of chromosomal aberrations. SNP-CGH also provides genetic information (haplotypes) of the locus undergoing aberration. Importantly, SNP-CGH has the capability of identifying copy-neutral LOH events, such as gene conversion, which cannot be detected with array-CGH.

In another embodiment, FISH is used to determine the DNA copy number alterations in a genome. Fluorescence in situ hybridization (FISH) is known to those of skill in the art (see Angerer, 1987 Meth. Enzymol., 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, or Cot-1 DNA is used to block non-specific hybridization.

In another embodiment, Southern blotting is used to determine the DNA copy number alterations in a genome. Methods for doing Southern blotting are known to those of skill in the art (see Current Protocols in Molecular Biology, Chapter 19, Ausubel, et al., Eds., Greene Publishing and Wiley-Interscience, New York, 1995, or Sambrook et al., Molecular Cloning: A Laboratory Manual, 2d Ed. vol. 1-3, Cold Spring Harbor Press, NY, 1989). In such an assay, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., genomic DNA from the same or related cell, tissue, organ, etc.) provides an estimate of the relative copy number of the target nucleic acid.

In one embodiment, amplification-based assays, such as PCR, are used to determine the DNA copy number alterations in a genome. In such amplification-based assays, the genomic region where a copy number alteration occurred serves as a template in an amplification reaction. In a quantitative amplification, the amount of amplification product will be proportional to the amount of template in the original sample. Comparison to appropriate controls provides a measure of the copy number of the genomic region.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y.

Real time PCR can be used in the methods of the invention to determine DNA copy number alterations. (See, e.g., Gibson et al., Genome Research 6:995-1001, 1996; Heid et al., Genome Research 6:986-994, 1996). Real-time PCR evaluates the level of PCR product accumulation during amplification. To measure DNA copy number, total genomic DNA is isolated from a sample. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A TaqMan-based assay also can be used to quantify a particular genomic region for DNA copy number alterations. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

In one embodiment, DNA sequencing is used to determine the DNA copy number alterations in a genome. Methods for DNA sequencing are known to those of skill in the art.

In one embodiment, karyotyping (such as spectral karyotyping, SKY) is used to determine the chromosomal structural aberrations in a genome. Methods for karyotyping are known to those of skill in the art. For example, for SKY, a collection of DNA probes, each complementary to a unique region of one chromosome, may be prepared and labeled with a fluorescent color that is designated for a specific chromosome. DNA amplification, deletion, translocations or other structural abnormalities may be determined based on fluorescence emission of the probes.

In certain embodiments, tumor samples from two or more genome-unstable animal models of the invention are analyzed for DNA copy number alterations, and the common genomic regions where the copy number alterations occurred in at least two of the samples are identified. Such recurrent DNA copy number alterations are of particular interest.

A minimum common region (MCR) of the recurrent DNA copy number alteration may be defined when copy number alterations of two or more samples are compared. In one embodiment, the MCR is defined by the boundaries of overlap between two samples, or by boundaries of a single tumor against a background of larger alterations in at least one other tumor.

Methods for determining MCRs is known in the art (see, e.g., D. R. Carrasco, et al., Cancer Cell 9 (4), 313 (2006); A. J. Aguirre, et al., Proc Natl Acad Sci USA 101 (24), 9067 (2004)). Briefly, a “segmented” dataset was generated by determining uniform copy number segment boundaries and then replacing raw log 2 ratio for each probe by the mean log 2 ratio of the segment containing the probe. A threshold representing minimal copy number alterations (CNAs) is then chosen to filter out noise. For example, the median log 2 ratio of a two-fold change for the platform may be chosen as a threshold. In an exemplary embodiment, the thresholds representing CNAs are +/−0.6 (Agilent 22K a-CGH platform) and +/−0.8 (Agilent 44K/244K a-CGH platform), and the width of MCR is less than 10 Mb.

The boundaries of MCRs can be mapped by any method that is known in the art, such as southern blotting, or PCR.

Genes and genetic elements located within an MCR are potentially related to human cancer and such genes and genetic elements can be subject to additional analyses to further characterize them. For example, a gene that is initially identified by array-CGH may be quantitatively amplified. Quantitative amplification of either the identified genomic DNA or the corresponding RNA can confirm DNA gain or loss. Alternatively, if the sequence encodes a protein, the mRNA level, protein level, or activity level of the encoded protein may be measured. An increase in RNA/protein/activity level, as compared to a control, confirms DNA amplification; a decrease in RNA/protein/activity level, as compared to a control, confirms DNA deletion.

The gene or genetic element identified through initial screening may also be re-sequenced to confirm amplification or deletion. Further, DNA sequencing and protein expression profiling may also be used to identify genetic mutations that may be associated with tumorigenesis.

In another aspect, the invention provides a method of identifying a chromosomal region of interest for the identification of a gene or genetic element that is potentially related to human cancer, comprising the step of identifying a chromosomal structural aberration in a population of cancer cells from a genome-unstable animal models of the invention. A chromosomal region containing the chromosomal structural aberration is a chromosomal region of interest for the identification of a gene or genetic element that is potentially related to human cancer.

In some embodiments, the chromosomal structural aberration is detected using karyotyping, such as SKY. In some embodiments, the method further comprises determining the DNA copy number alteration, as described above. A chromosomal region containing the both chromosomal structural aberration and a DNA copy number alteration is a chromosomal region of interest for the identification of a gene or genetic element that is potentially related to human cancer.

In another aspect, the invention provides a method of identifying a potential human cancer-related gene or genetic element, comprising the steps of (a) identifying a chromosomal region of interest as described herein; (b) identifying a gene or a genetic element within the chromosomal region of interest in the non-human animal, and (c) identifying a human gene or genetic element that corresponds to the gene or genetic element identified in step (b).

Additionally, many public and private databases provide cancer gene information (for example, Sanger's Cancer Gene Census, at http://www.sanger.ac.uk/genetics/CGP/Census), and the information may be used to map known cancer genes to a particular chromosomal region.

If a gene or a genetic element is found to be potentially relevant to human cancer, the corresponding human gene may be identified by homolog mapping, ortholog mapping, paralog mapping, among other methods. As used herein, a homolog is a gene related to a second gene by descent from a common ancestral DNA sequence, an ortholog is a gene in a different species that evolved from a common ancestral gene by speciation, and a paralogs is a gene related by duplication within a genome.

In one embodiment, human homologs are identified by using, for example, the NCBI homologene website, http://www.ncbi.nlm.nih.gov/entrez/query.fcgi?db=homologene.

In some embodiments, the method further comprises detecting a mutation in the identified non-human gene or genetic element. In another embodiment, a mutation in the corresponding human gene or genetic element is identified. In another embodiment, mutations in the both the non-human gene or genetic element and the human gene or genetic element are identified, and the mutations are compared.

In another aspect, the invention provides a method of identifying a potential human cancer-related gene or genetic element, comprising the steps of (a) detecting a DNA copy number alteration in a population of cancer cells from a non-human mammal, wherein the genome of the non-human mammal is engineered to produce genome instability, (b) identifying a gene or genetic element located within the boundaries of the copy number alteration detected in step (a), (c) identifying a human gene or genetic element that corresponds to the gene or genetic element identified in step (b) and that is located within the boundaries of a copy number alteration or of a chromosomal structural aberration in a human cancer cell. The human gene or genetic element identified in step (c) is a gene potentially related to human cancer.

Methods for detecting a copy number alteration or a chromosomal structural aberration have been described above in detail. Methods for identifying a gene or genetic element located within the boundaries of the copy number alteration are also described above in detail.

In one embodiment, a copy number alteration or a chromosomal structure aberration in the non-human animal model of the invention is compared with a copy number alteration or a chromosomal structural aberration in human cancer cell. A potentially relevant human cancer related gene or genetic element is identified based on synteny. Synteny describes the preserved order and orientation of genes between related species. Comparisons of non-human animal model and human cancer syntenic chromosomal regions may reveal the conserved nature of certain genetic modification in tumorgenesis.

The cross-species comparison based on synteny has several advantages. First is the ability to narrow the chromosomal regions of interest—certain genomic modification is more focal in one species than the other, and a cross-species comparison may eliminate such species-specific event. Second, a minimal common region (MCR) typically contains a number of genes; a cross-species comparison of syntenic regions allows an efficient way to reduce the gene numbers because the syntenic regions of the genome between non-human mammals (in particular, mice) and humans may be in relatively small portions. Genes located within syntenic MCRs may be highly relevant to human cancers.

In another aspect, the invention provides a method of identifying a potential human cancer-related gene or genetic element, comprising the steps of (a) detecting a chromosomal structural aberration in a population of cancer cells from a non-human mammal, wherein the genome of the non-human mammal is engineered to produce genome instability, (b) identifying a gene or genetic element located within the boundaries of the copy number alteration detected in step (a), (c) identifying a human gene or genetic element that corresponds to the gene or genetic element identified in step (b) and that is located within the boundaries of a copy number alteration or of a chromosomal structural aberration in a human cancer cell. The human gene or genetic element identified in step (c) is a gene potentially related to human cancer.

### 4. DIAGNOSIS AND METHODS OF TREATMENT

In one aspect, the present invention provides a method for identifying subjects with T-cell acute lymphoblastic leukemia (T-ALL) who may have a decreased or increased response to γ-secretase inhibitor therapy, based on the discovery that inactivation of FBXW7 is associated with human T-cell malignancy.

In one embodiment, the method for identifying subjects with T-ALL who may have a decreased response to a γ-secretase inhibitor therapy comprises: detecting in a cancer cell from the subject the expression level or activity level of FBXW7; a decreased expression/activity of FBXW7, as compared to a control, indicates that the subject may have a decreased response to a γ-secretase inhibitor therapy. The expression or activity level of NOTCH1 in the cancer cell may also be determined simultaneously; an increased expression/activity of NOTCH1, as compared to a control, further indicates that the subject may have a decreased response to a γ-secretase inhibitor therapy. Conversely, an increased expression/activity of FBXW7 (together with a decreased expression/activity of NOTCH1, optionally), as compared to a control, indicates that the subject may be sensitive to a γ-secretase inhibitor therapy.

γ-Secretase is a complex composed of at least four proteins, namely presenilins (presenilin 1 or -2), nicastrin, PEN-2, and APH-1. Several proteins have been identified as substrates for γ-secretase cleavage, include Notch and the Notch ligands Delta1 and Jagged2, ErbB4, CD44, and E-cadherin (Wong, G. T. et. al, J. Biol. Chem., Vol. 279, Issue 13, 12876-12882, Mar. 26, 2004). The cleavage of Notch by γ-secretase has been studied most extensively. Notch plays an evolutionarily conserved role in regulating cell growth and lineage specification particularly during embryonic development. Notch is activated by several ligands (Delta, Jagged, and Serrate) and is then proteolytically processed by a series of ligand-dependent and -independent cleavages. γ-Secretase catalyzes the terminal cleavage event (S3 cleavage), which releases a fragment known as the Notch intracellular domain (NICD). The NICD fragment then translocates to the nucleus where it acts as a nuclear transcription factor. As expected from its role in Notch S3 cleavage, γ-secretase inhibitors have been shown to block NICD production in vitro. In vivo, Notch function appears to be critical for the proper differentiation of T and B lymphocytes, and γ-secretase inhibitors reduce the thymocyte number and block thymocyte differentiation at an early stage in fetal thymic organ cultures.

The FBXW7 gene (also called hCDC4) encodes a key component of the E3 ubiquitin ligase that is implicated in the control of chromosome stability (Mao J. et. al, Nature 432, 775-779 (2004)). FBXW7 is responsible for binding the PEST domain of intracellular NOTCH1, leading to ubiquitination and degradation by the proteasome. Because there exists a statistically significant anti-correlation between PEST domain mutations in NOTCH1 and FBXW7 mutation in human T-ALL, T-ALL cells having a reduced expression/activity of FBXW7 will less likely to respond to γ-secretase inhibitors.

One of the recurring problems of cancer therapy is that a patient in remission (after the initial treatment by surgery, chemotherapy, radiotherapy, or combination thereof) may experience relapse. The recurring cancer in those patients is frequently resistant to the apparently successful initial treatment. In fact, certain cancers in patients initially diagnosed with the disease may be already resistant to conventional cancer therapy even without first being exposed to such treatment. γ-secretase inhibitor therapy can be physically exhausting for the patient. Side effects of secretase inhibitors include weight loss, changes in gastrointestinal tract architecture, accumulation of necrotic cell debris, dilation of crypts and infiltration of inflammatory cells, nausea, vomiting, weakness, diarrhea elevation in white blood cell count, and esophageal failure (Siemers E. et al, 2005 May-June; 28(3):126-32; Wong, G T. et al, J Biol Chem. 2004 Mar. 26; 279 (13):12876-82). Thus there is a need to determine whether a cancer patient may benefit from a chemotherapeutic treatment prior to the commencement of the treatment.

In one embodiment, a cancer patient is screened based on the expression level of FBXW7 and optionally, NOTCH1, in a cancer cell sample.

The expression level of FBXW7 or NOTCH1 may be measured by DNA level, mRNA level, protein level, activity level, or other quantity reflected in or derivable from the gene or protein expression data. For example, a genetic alteration may result in a decreased expression of FBXW7. Common genetic alterations include deletion of at lease one FBXW7 gene from the genome, or a mutation in at least one allele of an FBXW7 gene. The mutation may be a mis-sense mutation; a non-sense mutation; an insertion, deletion, or substitution of one or more nucleotides; a truncation from the 5′ terminal (either untranslated region or coding region), 3′ terminal (either untranslated region or coding region), or both; a substitution of one or more nucleotides in the 5′ untranslated region, 3′ untranslated region, coding region (which results in an amino acid change), or combinations of the three. Exemplary genetic alterations include a mutation in the third WD40 domain or the fourth WD40 domain of the FBXW7, G423V, R465C, R465H, R479L. R479Q, R505C and D527G mutations. A genetic alteration may also result in an increased expression of NOTCH1, such as translocation or copy number amplification of NOTCH1 gene.

The mRNA level of FBXW7 or NOTCH 1 may be measured using any art-known method, such as PCR, northern blotting, RNase Protection Assay, or microarray hybridization. For example, Real-time polymerase chain reaction, also called quantitative real time PCR (QRT-PCR) or kinetic polymerase chain reaction, is widely used in the art to measure mRNA level of a target gene. The QRT-PCR procedure follows the general pattern of polymerase chain reaction, but the DNA is quantified after each round of amplification. Two common methods of quantification are the use of fluorescent dyes that intercalate with double-strand DNA, and modified DNA oligonucleotide probes that fluoresce when hybridized with a complementary DNA. QRT-PCR can be combined with reverse transcription polymerase chain reaction to quantify low abundance messenger RNA (mRNA), enabling one to quantify relative gene expression at a particular time, or in a particular cell or tissue type.

The expression level of FBXW7 or NOTCH1 may also be measured by protein level using any art-known method. Traditional methodologies for protein quantification include 2-D gel electrophoresis, mass spectrometry and antibody binding. Frequently used methods for assaying target protein levels in a biological sample include antibody-based techniques, such as immunoblotting (western blotting), immunohistological assay, enzyme linked immunosorbent assay (ELISA), radioimmunoassay (RIA), or protein chips. Gel electrophoresis, immunoprecipitation and mass spectrometry may be carried out using standard techniques. Additionally, NOTCH1 expression may be measured by detection of cleaved, intranuclear (ICN) form of NOTCH1 protein in cells.

The expression level of FBXW7 or NOTCH1 may also be measured by the activity level of the gene product using any art-known method, such as transcriptional activity of NOTCH1 or ligase activity of FBXW7. For example, NOTCH1 activity may be measured by a increased binding of ICN of NOTCH1. Alternatively, the expression level of a transcriptional downstream target of NOTCH1 may be measured as an indicator of NOTCH1 activity, such as c-Myc, PTCRA, Hes1, etc.

In certain embodiments, it is useful to compare the expression/activity level of FBXW7 or NOTCH1 to a control. The control may be a measure of the expression level of FBXW7 or NOTCH1 in a quantitative form (e.g., a number, ratio, percentage, graph, etc.) or a qualitative form (e.g., band intensity on a gel or blot, etc.). A variety of controls may be used. Levels of FBXW7 or NOTCH1 expression from a non-cancer cell of the same cell type from the subject may be used as a control. Levels of FBXW7 or NOTCH1 expression from the same cell type from a healthy individual may also be used as a control. Alternatively, the control may be expression levels of FBXW7 or NOTCH1 from the individual being treated at a time prior to treatment or at a time period earlier during the course of treatment. Still other controls may include expression levels present in a database (e.g., a table, electronic database, spreadsheet, etc.) or a pre-determined threshold.

The present invention further discloses methods of treating a T-ALL subject who will likely be sensitive a treatment with γ-secretase inhibitors (identified using the methods described above), comprising administering to the patients a γ-secretase inhibitor. γ-secretase inhibitors are known in the art, exemplary γ-secretase inhibitors include LY450139 Dihydrate and LY411575.

The present invention further discloses methods of treating a T-ALL subject who will has a decreased expression/activity of FBXW7 (identified using the methods described above) with an agent that increases the expression/activity of FBXW7. The agent may be a recombinant FBXW7 protein or a functionally active fragment or derivative thereof, a nuclei acid that encodes FBXW7 protein or a functionally active fragment or derivative thereof, or an agent that activates FBXW7. A “functionally active” PBXW7 fragment or derivative exhibits one or more functional activities associated with a full-length, wild-type FBXW7 protein, such as antigenic or immunogenic activity, ability to bind natural cellular substrates, etc. The functional activity of FBXW7 proteins, derivatives and fragments can be assayed by various methods known to one skilled in the art (Current Protocols in Protein Science, Coligan et al., eds., John Wiley & Sons, Inc., Somerset, N.J. (1998)).

In another aspect, the present invention provides a method for identifying subject with T-ALL who may benefit from treatment with a phosphatidylinositol 3-kinase (PI3K) pathway inhibitor, based on the discovery that PTEN inactivation is associated with human T-cell malignancy.

PTEN has been characterized as a tumor suppressor gene that regulates cell cycle. PTEN functions as a phosphodiesterase and an inhibitor of the PI3K/AKT pathway, by removing the 3′ phosphate group of phosphatidylinositol (3,4,5)-trisphosphate (PIP₃). When PTEN is inactivated, increased production of PIP₃ activates AKT (protein kinase B). The AKT pathway promotes tumor progression by enhancing cell proliferation, growth, survival, and motility, and by suppressing apoptosis. AKT is activated by two phosphorylation events catalyzed by the phosphoinositide dependent kinase PDK1, an enzyme that is activated by PI3K.

In one embodiment, the method for identifying subject with T-ALL who may benefit from treatment with a PI3K pathway inhibitor comprises: detecting in a tumor cell from the subject the expression level or activity level of PTEN. A decreased expression/activity of FBXW7, as compared to a control, indicates that the subject may benefit from a PI3K inhibitor therapy.

The phospho-AKT level in the cancer cell from the subject may also be determined simultaneously; an increased phospho-AKT level, as compared to a control, further indicates that the subject may benefit from a PI3K inhibitor therapy.

The expression level of PTEN may be measured by DNA level, mRNA level, protein level, activity level, or other quantity reflected in or derivable from the gene or protein expression data. For example, a genetic alteration may result in a decreased expression of PTEN. Common genetic alterations include deletion of at least one PTEN gene from the genome, or a mutation in at least one allele of a PTEN gene. The mutation may be a mis-sense mutation; a non-sense mutation; an insertion, deletion, or substitution of one or more nucleotides; a truncation from the 5′ terminal (either untranslated region or coding region), 3′ terminal (either untranslated region or coding region), or both; a substitution of one or more nucleotides in the 5′ untranslated region, 3′ untranslated region, coding region (which results in an amino acid change), or combinations of the three.

The expression level of PTEN may also be measured by mRNA level using any method known in the art, such as PCR, Northern blotting, RNase Protection Assay, and microarray hybridization.

The expression level of PTEN may also be measured by protein level using any method known in the art, such as 2-D gel electrophoresis, mass spectrometry and antibody binding

The expression level of PTEN may also be measured by the activity level of PTEN using any art-known method, such as measuring the phosphatase activity. Additionally, the expression or activity of other proteins involved in the PI3K/AKT pathway may also be measured as a proxy for PTEN activity. For example, the phospho-AKT level in a cell generally reflects the PTEN activity, therefore may be measured as a marker for PTEN activity.

In certain embodiments, a control may be used to compare the expression/activity level of PTEN. As described in detail above, a control may be derived from a non-cancer cell of the same type from the subject, same cell type from a healthy individual, a predetermined value, etc.

The present invention further discloses methods of treating a T-ALL subject who may benefit from a treatment with PI3K inhibitors (identified using the methods described above), comprising administering to the patients a PI3K inhibitor. PI3K inhibitors are well know in the art (e.g., Pinna, L A and Cohen, P T W (eds.) Inhibitors of Protein Kinases and Protein Phosphates, Springer (2004) and Abelson, J N, Simon, M I, Hunter, T, Sefton, B M (eds.) Methods in Enzymology, Volume 201: Protein Phosphorylation, Part B: Analysis of Protein Phosphorylation, Protein Kinase Inhibitors, and Protein Academic Press (2007)).

The present invention further discloses methods of treating a T-ALL subject who will has a decreased expression/activity of PTEN (identified using the methods described above) with an agent that increases the expression/activity of PTEN. The agent may be a recombinant PTEN protein or a functionally active fragment or derivative thereof, a nuclei acid that encodes PTEN protein or a functionally active fragment or derivative thereof, or an agent that activates PTEN.

In another aspect, the invention provides a method of assessing whether a subject is afflicted with cancer or at risk for developing cancer, comprising: determining the expression or activity level of at least one cancer gene or candidate cancer gene located in an amplified MCR in Table 1 in a biological sample from the subject. An increase in the expression or activity the gene, as compared to a control, indicates that the subject is afflicted with cancer or at risk for developing cancer. Alternatively, if there is a decrease in the expression or activity of a cancer gene or candidate cancer gene located in a deleted MCR in Table 1, as compared to a control, the decreased expression or activity level also indicates that the subject is afflicted with cancer or at risk for developing cancer.

In another aspect, the invention provides a method of assessing whether a subject is afflicted with cancer or at risk for developing cancer, the method comprising: determining the copy number of at least one amplified minimal common region (MCR) listed in Table 1 in a biological sample from the subject. An increased copy number of the MCR in the sample, as compared to the normal copy number of the MCR, indicates that the subject is afflicted with cancer or at risk for developing cancer. Alternatively, a decreased copy number of a deleted MCR (also listed in Table 1) in the sample, as compared to the normal copy number of the MCR, also indicates that the subject is afflicted with cancer or at risk for developing cancer. The normal copy number of an MCR is typically one per chromosome.

In another aspect, the invention provides a method for monitoring the progression of cancer in a subject, the method comprising: a) determining in a biological sample from the subject at a first point in time, the expression or activity level of a cancer gene or a candidate cancer gene listed in Table 1; b) repeating step a) at a subsequent point in time; and c) comparing the expression or activity of the gene in steps a) and b), and therefrom monitoring the progression of cancer in the subject.

In another aspect, the invention provides a method of assessing the efficacy of a test agent for treating a cancer in a subject, comprising: a) determining the expression or activity level of at least one cancer gene or a candidate cancer gene located in an amplified MCR in Table 1 in a biological sample from the subject in the presence of the test agent; and b) determining the expression or activity level of the gene in a biological sample from the subject in the absence of the test agent. A decreased expression or activity of the gene in step (a), as compared to that of (b), is indicative of the test agent's potential efficacy for treating the cancer in the subject. Alternatively, if the test agent increases the expression or activity of at least one cancer gene or a candidate cancer gene located in a deleted MCR in Table 1, the test agent is also potentially effective for treating the cancer in a subject.

In another aspect, the invention provides a method of assessing the efficacy of a therapy for treating cancer in a subject, the method comprising: a) determining the expression or activity level of at least one cancer gene or a candidate cancer gene located in an amplified MCR in Table 1 in a biological sample from the subject prior to providing at least a portion of the therapy to the subject; and b) determining the expression or activity level of the gene in a biological sample from the subject following provision of the portion of the therapy. A decreased expression or activity of the gene in step (a), as compared to that of (b), is indicative of the therapy's efficacy for treating the cancer in the subject. Alternatively, if the therapy increases the expression or activity of at least one cancer gene or a candidate cancer gene located in a deleted MCR in Table 1, the therapy is also potentially effective for treating the cancer in a subject.

In another aspect, the invention provides a method of treating a subject afflicted with cancer comprising administering to the subject an agent that decreases the expression or activity level of at least one cancer gene or candidate cancer gene located in am amplified MCR in Table 1. Alternatively, the invention provides a method of treating a subject afflicted with cancer comprising administering to the subject an agent that increases the expression or activity level of at least one cancer gene or candidate cancer gene located in a deleted MCR in Table 1.

In certain embodiments, the agent is an antibody, or its antigen-binding fragment thereof, that specifically binds to a cancer gene or candidate cancer gene listed in Table 1. Optionally, the antibody may be conjugated to a toxin, or a chemotherapeutic agent.

Alternatively, the agent may be an RNA interfering molecule (such as an shRNA or siRNA molecule) that inhibits expression of a cancer gene or candidate cancer gene in an amplified MCR in Table 1, or an antisense RNA molecule complementary to a cancer gene or candidate cancer gene in an amplified MCR in Table 1.

Alternatively, the agent may be a peptide or peptidomimetic, a small organic molecule, or an aptamer.

Preferrably, the agent is administered in a pharmaceutically acceptable formulation.

In another aspect, the invention provides a method of assessing whether a subject is afflicted with cancer or at risk for developing cancer, the method comprising: determining the copy number of at least one minimal common region (MCR) listed in Table 5 in a biological sample from the subject. A change of copy number of the MCR in the sample, as compared to the normal copy number of the MCR, indicates that the subject is afflicted with cancer or at risk for developing cancer. The normal copy number of an MCR is typically one per chromosome.

In certain embodiments, the cancer is lymphoma. In certain embodiments, the lymphoma is T-ALL.

In another aspect, the invention provides a method of assessing whether a subject is afflicted with cancer or at risk for developing cancer, by comparing the copy number of an MCR, identified using a genome-unstable non-human mammal model (including a genome-unstable mouse model of the invention), with the normal copy number of the MCR. The normal copy number of an MCR is typically one per chromosome.

### EXAMPLES

### Example 1

### Generation and Characterization of Murine T Cell Lymphomas with Highly Complex Genomes

In this example, we created a murine lymphoma model system that combines the genome-destabilizing impact of Atm deficiency and telomere dysfunction to effect T lymphomagenesis in a p53-dependent manner.

We interbred mTerc Atm p. 53 heterozygous mice and maintained them in pathogen-free conditions. We intercrossed the null alleles of mTerc, Atm and p53 to generate various genotypic combinations from this “triple”-mutant colony (for simplicity, hereafter designated as “TKO” for all genotypes from this colony).

We monitored animals for signs of ill-health every other day. Moribund animals were euthanized and subjected to complete autopsy; mice found dead were subject to necropsy specifically for signs of lymphoma. We performed all animal uses and manipulations according to approved IACUC protocol. Tumors were harvested from TKO mice and partitioned in the following manner. One section was snap-frozen for DNA and RNA extraction, a second portion was processed for histology, and the remaining portion was disaggregated for in vitro culture. Suspensions of tumor cells were maintained in RPMI supplemented with 50 μM beta-mercaptoethanol, 10% Cosmic Calf serum (HyClone), 0.5 ng/ml recombinant IL-2, and 4 ng/ml recombinant IL-7 (both from Peprotech). Tumor cells were immunostained with antibodies against CD4, CD8, CD3, and B220/CD45R (eBioscience) and subjected to FACS analysis.

We prepared DNA frozen tumors with the PureGene kit according to manufacturer's instructions (Gentra Systems). We prepared RNA by an initial extraction with Trizol (Invitrogen) according to the manufacturer's instructions. Pelleted total RNA was then digested with RQ1 DNase (Promega) and subsequently purified through RNA purification columns (Gentra). Proteins were obtained either from cell lines or tumor pieces by dis-aggregation in lysis buffer (according to Cell Signaling Technology) followed by sonication in a bath sonicator for 30 s. Lysates were clarified by centrifugation prior to quantification according to manufacturer's instructions (BioRad Protein Assay) and separation on 4-12% NuPage gels (Invitrogen).

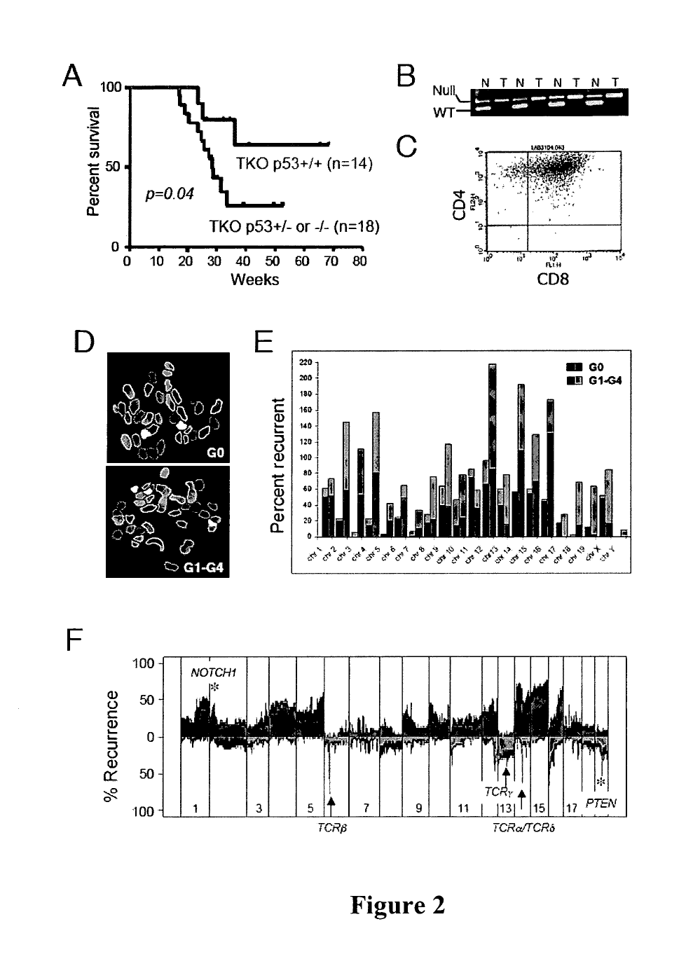

We found that TKO mice which are p53^{+/-} or p53^{-/-} succumbed to lethal lymphoma with shorter latency and higher penetrance relative to TKO animals wildtype for p53 (FIG. 2A). Moreover, lymphomas from TKO mice heterozygous for p53 showed reduction to homozygosity in 14 specimens (out of 15 specimens examined) (FIG. 2B), indicating strong genetic pressure to inactivate p53 during lymphomagenesis in this context. Phenotypically, these TKO tumors resembled lymphomas in the conventional Atm−/− mouse model with effacement of thymic architecture by CD4+/CD8+ (less commonly CD4−/CD8− or mixed single/double positive) lymphoma cells (FIG. 2C). Taken together, the genetic and molecular observations strongly suggest that an Atm-independent p53-dependent telomere checkpoint is operative to constrain lymphoma development.

To quantify chromosomal rearrangements, we used Spectral Karyotype (SKY) analyses according to the following protocol. Metaphase preparations were typically obtained within 48 hours of establishment, although in a few instances establishment of the cell line was required to obtain good quality metaphases. Harvested cells were incubated in 105 mM KCl hypotonic buffer for 15 min prior to fixation in 3:1 methanol-acetic acid. Spectral karyotyping was done using the SkyPaint Kit and SkyView analytical software (Applied Spectral Imaging, Carlsbad, Calif.) according to manufacturer's protocols. Chromosome aberrations were defined using the rules from the Committee on Standard Genetic Nomenclature for Mice. T-test comparison between G0 and G1-G4 cytogenetics is based on 90 SKY profiles each set (ten metaphase spreads for each of TKO lymphomas).

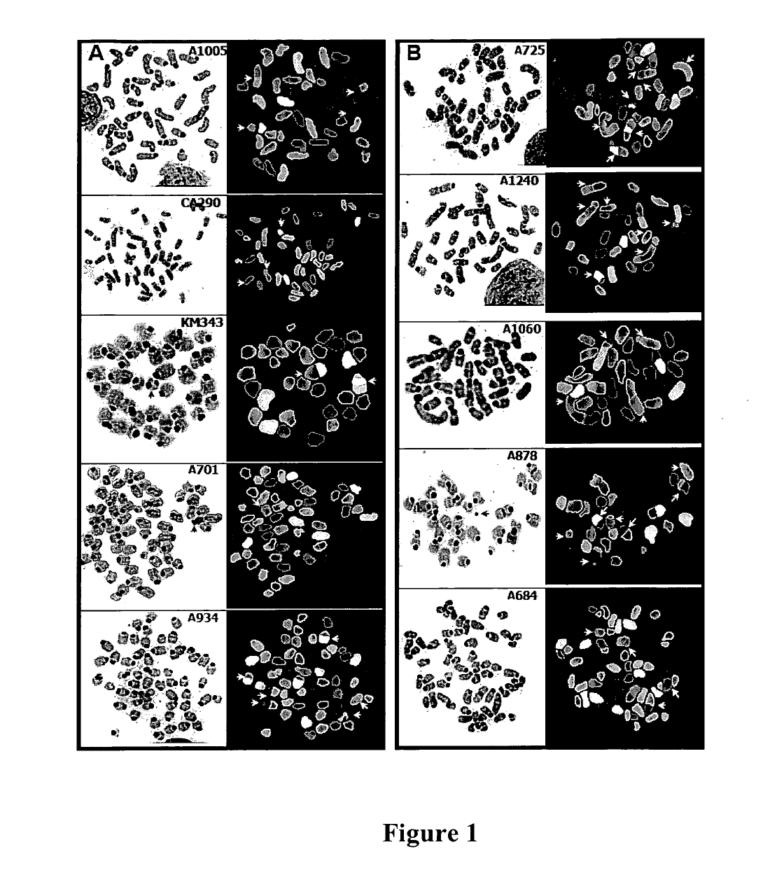

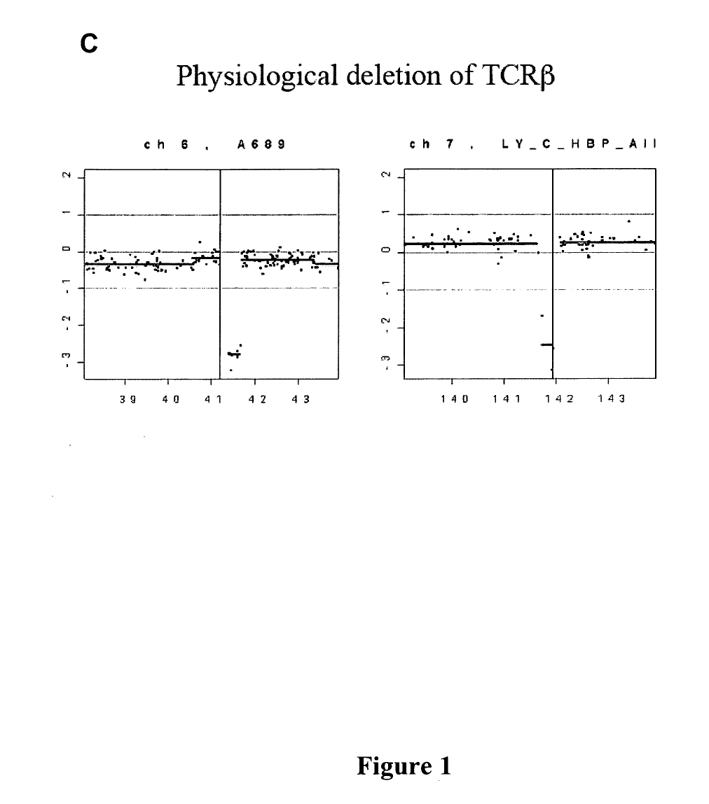

FIG. 1, FIG. 2D, and Table 3 summarize the SKY analyses of chromosomal rearrangement in 9 telomere deficient (G1-G4 mTerc^{-/-}) TKO lymphomas and 9 telomere intact (G0 mTerc^{+/+} or mTerc^{+/-}) TKO lymphomas. Relative to G0 tumors, G1-G4 TKO lymphomas displayed an overall greater frequency of chromosome structural aberrations of various types (0.34 versus 0.09 per chromosome, respectively, p<0.0001, t test) including a multitude of multi-centric chromosomes, non-reciprocal translocations (NRTs), p-p robertsonian-like translocations of homologous and/or non-homologous chromosomes, p-q fusions, and q-q fusions. When examined on a chromosome-by-chromosome basis, several chromosomes (specifically, 2, 6, 8, 14, 15, 16, 17, and 19) were involved in significantly more dicentric and robertsonian-like rearrangement events in G1-G4 relative to G0 TKO tumors (p<0.05; t test; FIG. 2E). Without being bound by a particular theory, the recurrent non-random nature of these chromosomal rearrangements in the TKO model may provide adaptive mechanisms to tolerate telomere dysfunction and/or play causal roles in lymphoma development (e.g., chromosome 2, see below).

### Example 2

### TKO Lymphomas Harbor Genomic Alterations Syntenic to Those in Human T Cell Malignancy

To assess the degree of syntenic overlap in the murine lymphoma-prone TKO instability model and in human T-ALL and other cancers, we applied and integrated multiple genome analysis technologies to survey cancer-associated alterations for comparison with T-ALL and a diverse set of major human cancers.

Synteny describes the preserved order and orientation of genes between species. Disruption of synteny, caused by chromosome rearrangement, is an indication of divergent evolution. Comparisons of TKO mouse model and human T-ALL syntenic chromosomal regions may reveal the conserved nature of certain genetic modification in tumorigenesis.

Because TKO lymphomas harbored a large number of complex nonreciprocal translocations (NRTs), we sought to determine whether these genome-unstable tumors possess increased numbers of recurrent amplifications and deletions. To this end, we compiled high-resolution genome-wide array-CGH profiles for 35 TKO tumors (Table 3) and 26 human T-ALL cell lines and tumors (Tables 4A and 4B) for comparison.

T-ALL cell lines used in this example, and in Examples 3-7 are listed in Table 4A. A subset was subjected to both array-CGH (described in detail below) and re-sequencing, as indicated.

We used two cohorts of clinical human T-ALL samples in this example. A cohort of 8 samples (Table 4B) comprised of cryopreserved lymphoblasts or lymphoblast cell lysates, obtained with informed consent and IRB approval at the time of diagnosis from pediatric patients with T-ALL treated on Dana-Farber Cancer Institute study 00-001. We subjected these samples to genome-wide array-CGH profiling.

For genome-wide array-CGH profiling, we used the following protocol. Genomic DNA processing, labeling and hybridization to Agilent CGH arrays were performed as per manufacturer's protocol (http://www.home.agilent.com/agilent/home.jspx). Murine tumors were profiled against individual matched normal DNA (e.g., non-tumor cell of the same cell type from the same individual) or, when not available, pooled DNA of matching strain background. Labeled DNAs were hybridized onto 44K or 244K microarrays for mouse, and 22K or 44K microarrays for human. The Mouse 44K array contained 42,404 60-mer elements for which unique map positions were defined (National Center for Biotechnology Information, Mouse Build 34). The median interval between mapped elements was 21.8 kb, 97.1% of intervals of <0.3 megabases (Mb), and 99.3% are <1 Mb. The 244K array contained 224,641 elements for which unique map positions were defined based on the same mouse genome build. The Human 22K array contained 22,500 elements designed for expression profiling for which 16,097 unique map positions were defined with a median interval between mapped elements of 54.8 kb. The Human 44K microarray contained 42,494 60-mer oligonucleotide probes for which unique map positions were defined (National Center for Biotechnology Information, Human Build 35). The 244K array contained 226,932 60-mer oligonucleotide probes for which unique map positions were defined based on the same human genome build.

Profiles generated on 244K density arrays were extracted for the same 42K probes on the 44K microarrays to allow combination of profiles generated on the two different platforms. Fluorescence ratios of scanned images were normalized and calculated as the average of two paired (dye swap), and copy number profile was generated based on Circular Binary Segmentation, an algorithm that uses permutation to determine the significance of change points in the raw data (A. B. Olshen, et al., Biostatistics 5 (4), 557 (2004)).

TKO profiles revealed marked genome complexity with all chromosomes exhibiting recurrent CNAs—both regional and focal in nature (FIG. 2F). Many CNAs were highly recurrent, observed in more than 40% of samples (e.g., amplicons targeting distinct regions on mouse chromosomes 1, 2, 3, 4, 5, 9, 10, 12, 14, 15, 16, and 17; and deletions on 6, 11, 12, 13, 14, 16 and 19). These patterns of genomic alteration corresponded well with the SKY analyses showing predominant involvement of these chromosomes in rearrangement events. Attesting to the robustness and resolution of this platform, highly recurrent physiological deletions of the T cell receptor (Tcr) loci were readily detected (FIG. 2F, arrows) as expected for clonal CD4/CD8-positive T-cells, e.g., chromosome 6 Tcrβ locus sustained focal deletion in 28/35 tumors, as well as focal deletions of chromosome 14 Tcrα/Tcrβ locus and chromosome 13 Tcrδ locus (FIG. 1C; FIG. 2F).

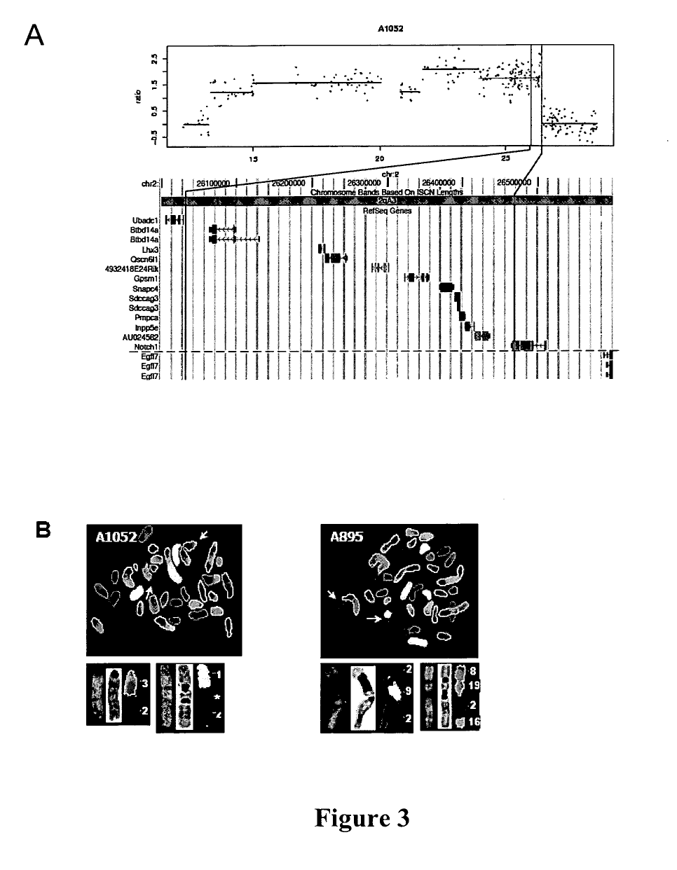

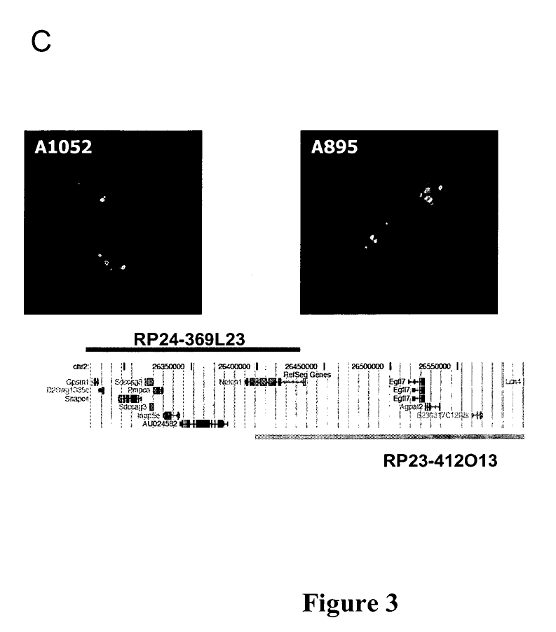

The pathogenetic relevance of these recurrent genomic events, and of this instability model, is supported by integrated array-CGH and SKY analyses of a high amplitude genomic event on chromosome 2 in several independent TKO tumors. These CNAs shared a common boundary defined by array-CGH and contained a recurrent NRT involving the A3 band of chromosome 2 with different partner chromosomes by SKY (FIG. 3).

### Example 3

### Frequent NOTCH1 Rearrangement in TKO Mouse Model

For further comparison of genomic events in the TKO model and in human T-All, we used a separate series of 38 human clinical specimens (Table 4C) for re-sequencing of NOTCH 1, FBXW7 and PTEN (see Examples 5-6). These T-ALL samples were collected from 8 children and adolescents diagnosed at the Royal Free Hospital, London, and 30 adult patients enrolled in the MRC UKALL-XII trial. Appropriate informed consent was obtained from the patients (if over 18 years of age) or their guardians (if under 18 years), and the study had Ethics Committee approval.

1. HPLC and Sequencing. Gene mutation status was established by denaturing high-performance liquid chromatography (see, e.g., M. R. Mansour, et al., Leukemia 20 (3), 537 (2006)), and by bidirectional sequencing. Briefly, genomic DNA was extracted using the Qiagen (Hilden, Germany) genomic purification kit. PCR primers were designed to amplify exons and flanking intronic sequences. PCR amplification and direct sequencing were done according to art-known methods (for details, see H. Davies, et al., Cancer Res 65 (17), 7591 (2005)). Sequence traces were analysed using a combination of manual analysis and software-based analyses, where deviation from normal is indicated by the presence of two overlapping sequencing traces (indicating the presence of one normal allelic and one mutant allelic DNA sequence), or the presence of a single sequence trace that deviates from normal (indicating the presence of only a mutant DNA allele). All variants were confirmed by bidirectional sequencing of a second independently amplified PCR product.

2. Expression profiling. Biotinylated target cRNA was generated from total sample RNA from a TKO model and hybridized to mouse oligonucleotide probe arrays against normal control murine thymus RNA (Mouse Development Oligo Microarray, Agilent, Palo Alto, Calif.) according to manufacturer's protocols. Expression values for each gene were mapped to genomic positions based on National Center for Biotechnology Information Build 34 of the mouse genome.

3. Real-Time PCR. To confirm genetic loci, Real-time PCR was performed with a Quantitect SYBR green kit (Qiagen USA, Valencia, Calif.) using 2 ng DNA from each tumor run in triplicate, on Applied Biosystems or Stratagene MX3000 realtime thermocyclers. Each triplicate run was performed twice; quantification was performed using the standard curve method and the average fold change for the combined run was calculated. Primer sequences are listed in Table 8.

4. Western Blotting. Western blots were performed on clarified tumor lysates on PVDF membranes using the following antibodies: PTEN (9552), Akt (9272), phospho-Akt (9271), Notch1, activated Notch1 Val1744 (2421) (Cell Signaling Technology, Ipswich, Mass.), and tubulin (Sigma Chemical, St. Louis, Mo.), according to the manufacturer's instructions and developed with HRP-labeled secondary antibodies (Pierce; Rockford, Ill.) and enhanced chemiluminescent substrate.

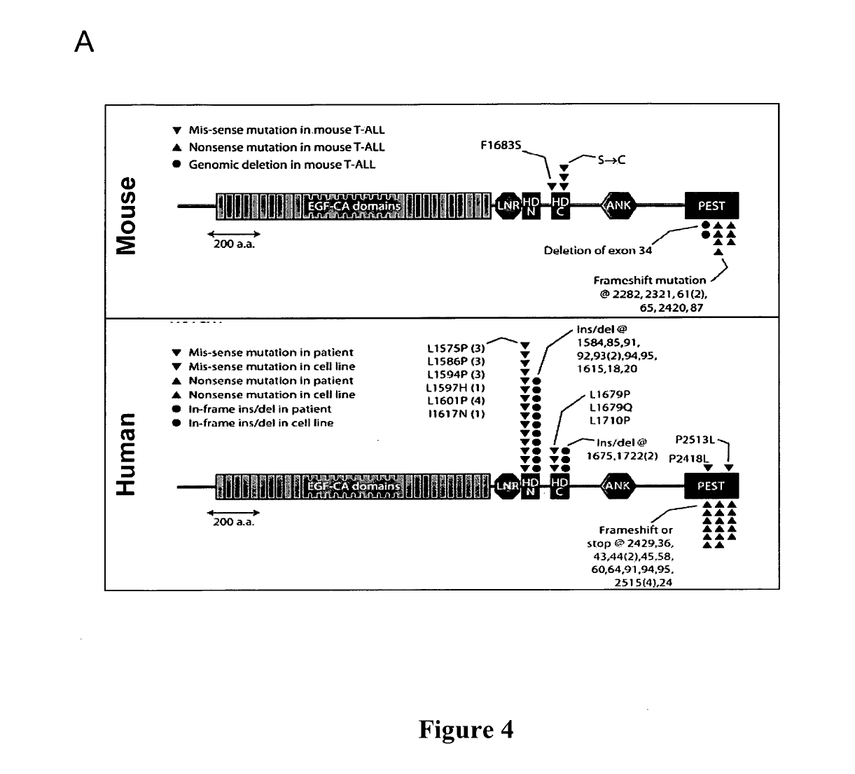

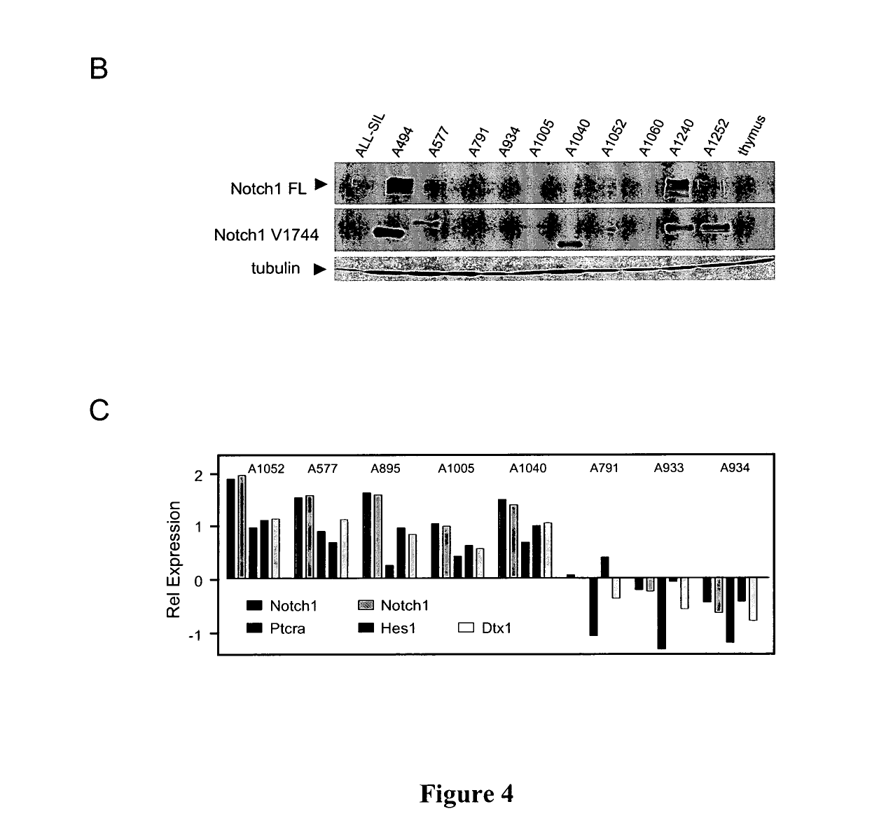

5. Common Boundary Analysis of NOTCH1. Detailed structural analysis of the common boundary of CNAs revealed Notch1 locus alterations with rearrangement close to the 3′ region of the Notch1 gene in four TKO tumors, and focal amplifications encompassing Notch1 in two additional tumors (FIG. 3; data not shown). Notch1 activation by C-terminal structural alteration and point mutations is a signature event of human T-ALL (see, A. P. Weng, et al., Science 306 (5694), 269 (2004), F. Radtke, et al., Nat Immunol 5 (3), 247 (2004), L. W. Ellisen, et al., Cell 66 (4), 649 (1991)). Although the structure of the rearrangements in the TKO samples did not precisely mirror NOTCH1 translocations in human T-ALL (L. W. Ellisen, et al., Cell 66 (4), 649 (1991)), their common shared boundary involving Notch1 suggested potential relevance of the TKO tumors. Accordingly, we performed Notch1 re-sequencing in several TKO lymphomas without evidence of genomic rearrangement at this locus and uncovered truncating insertion/deletion mutations and non-conservative amino acid substitutions in the Notch1 PEST and heterodimerization (HD) domains, as well as one case of an intragenic 379 by deletion within exon 34 encoding the PEST domain (sample A1040) (FIG. 4A; Table 3). This mutation spectrum is similar to that observed in human T-ALL, as the PEST and HD domains are two hot spots of NOTCH1 mutation (FIG. 4A, see below) (A. P. Weng, et al., Science 306 (5694), 269 (2004). Biochemically, various types of genomic rearrangements, intragenic deletions and mutations promoted activation of Notch1, as evidenced by Western blot assays designed to detect full-length protein and the active cleaved form (V1744) of Notch1 proteins (FIG. 4B) as well as by transcriptional profiles showing up-regulation of several Notch1 transcriptional targets including Ptcra, Hes1, Dtx1, and Cd3e that correlated well with mRNA levels of Notch1 (F. Radtke, et al., Nat Immunol 5 (3), 247 (2004)) (FIG. 4C).

### Example 4

### Determining Synteny Across Species by Ortholog Mapping of Genes within the Minimal Common Regions of Copy Number Alterations

In this Example, We further assessed the CNAs in the TKO mouse model by defining and characterization the minimal common regions of CNAs.

Synteny describes the preserved order and orientation of genes between species. Disruption of synteny, caused by chromosome rearrangement, is an indication of divergent evolution. Comparisons of TKO mouse model and human T-ALL syntenic chromosomal regions may reveal the conserved nature of certain genetic modification in tumorigenesis.

The observation of physiological deletion of TCR loci and human-like pattern of Notch1 genomic and mutational events prompted us to assess the extent to which the highly unstable genome of the TKO model engendered CNAs targeting loci syntenic to CNAs in human T-ALL using ortholog mapping of genes resident within the minimal common regions (MCRs) of copy number alterations.

1. Definition of MCRs. To facilitate this comparison, we first defined the MCRs in TKO genome by an established algorithm (see, e.g., D. R. Carrasco, et al., Cancer Cell 9 (4), 313 (2006); A. J. Aguirre, et al., Proc Natl Acad Sci USA 101 (24), 9067 (2004)) with criteria of CNA width<=10 Mb and amplitude>0.75 (log 2 scale). Briefly, a “segmented” dataset was generated by determining uniform copy number segment boundaries according to the method of Olshen (A. B. Olshen, et al., Biostatistics 5 (4), 557 (2004) and then replacing raw log 2 ratio for each probe by the mean log 2 ratio of the segment containing the probe. For 22K and 44K profiles, thresholds representing minimal CNA were chosen at ±0.15 and ±0.3, respectively.

Thresholds representing CNAs were chosen at ±0.4 and ±0.6, respectively. Higher thresholds were used for 44K profiles comparing to 22K profiles to adjust for signal-to-noise detection difference in platform performance. For examples 3-6, w selected minimal common region (MCR) by requiring at least one sample to show an extreme CNA event, defined by a log 2 ratio of ±0.60 and ±0.75 for 22K and 44K profiles, respectively, and the width of MCR is less than 10 Mb.

2. Homolog Mapping. We identified human homologs of genes identifies in regions of chromosomal structural alteration of CNAs within mouse TKO MCRs using NCBI HOMOLOGENE database. In parallel, we identified CNAs in seven human tumor datasets (pancreatic, glioblastoma, melanoma, lung, colorectal and multiple myeloma). The human homolog gene list was then used to merge with genes within CNAs of each of the seven human tumor datasets.

3. Cancer Gene Mapping. For cancer gene mapping, the mouse homologs were obtained based on Sanger's Cancer Gene Census⁵⁵ (http://www.sanger.ac.uk/genetics/CGP/Census). The mouse cancer genes were then mapped to TKO's MCRs.

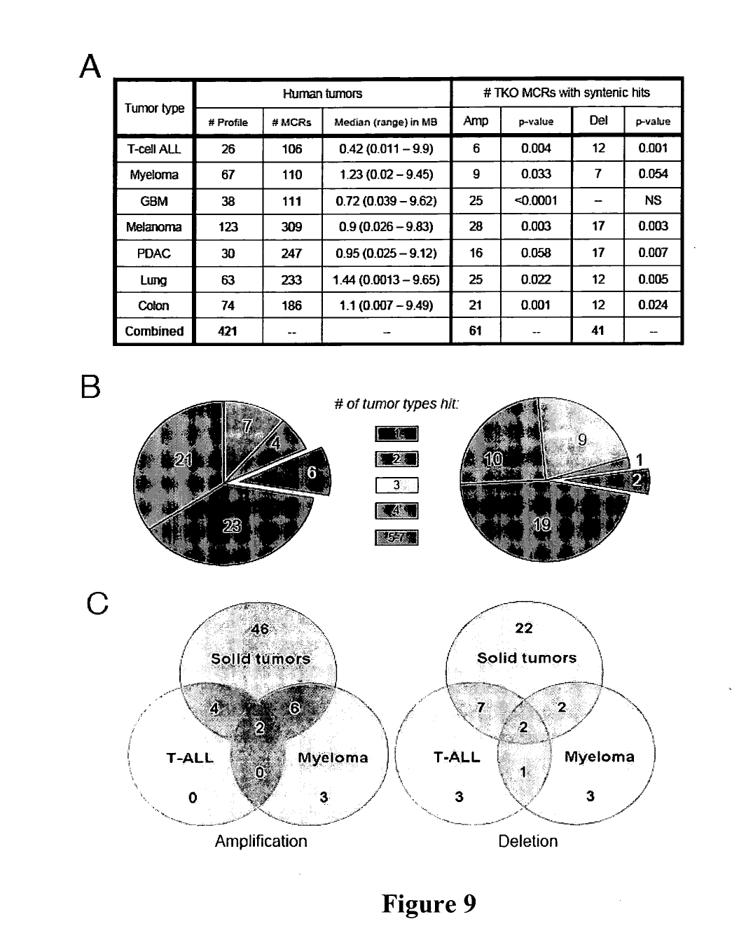

We obtained a list of 160 MCRs with average sizes of 2.12 Mb (0.15-9.82 Mb) and 2.33 Mb (0.77-9.6 Mb) for amplifications and deletions, respectively (Table 5). This frequency of genomic alterations is comparable to that of most human cancer genomes (e.g. FIG. 9A) and significantly above the typical 20 to 40 events detected in most genetically engineered ‘genome-stable’ murine tumor models (e.g., R. C. O'Hagan, et al., Cancer Res 63 (17), 5352 (2003); N. Bardeesy, et al., Proc Natl Acad Sci USA 103 (15), 5947 (2006); M. Kim, et al., Cell 125 (7), 1269 (2006); L. Zender, et al., Cell 125 (7), 1253 (2006)). When compared to similarly defined MCR list in human T-ALL, 18 of the 160 MCRs (11%) overlapped with defined genomic events present in the human counterpart (Table 1).

In Table 1, each murine TKO MCR with syntenic overlap with an MCR in the human T-ALL dataset is listed, separated by amplification and deletion, along with its chromosomal location (Cytoband/Chr) and base number (Start and End, in Mb). The minimal size of each MCR is indicated in bp. Peak ratio refers to the maximal log 2 array-CGH ratio for each MCR. Rec refers to the number of tumors in which the MCR was defined. Cancer genes and candidate cancer genes located in the amplified MCRs and deleted MCRs are also listed. The NCBI accession numbers and identification numbers for these cancer genes and candidate cancer genes are listed in Table 9.

To calculate the statistic significance of MCR overlap between mouse TKO and each of the human cancers of different histological types, we implemented a permutation test to determine the expected frequency of achieving the same degree of overlap between two genomes by chance alone. Specifically, we randomly generated simulated mouse genome containing the same number and sizes of amplification MCRs in the corresponding chromosomes as the actual TKO genome a similar set was created for each of the human cancer genomes. The number of overlapping amplifications between mouse and each human genome was calculated and stored. This simulation process was repeated 10,000 times. The p value for significance of amplification overlap was then calculated by dividing the frequency of randomly achieving the same or greater degree of overlap as actually observed during the 10,000 permutations by 10,000. p values for deletion overlap were calculated in a similar fashion.

We concluded that this degree of overlap was not by chance. First, statistic significance (p=0.001 and 0.004 for deletions and amplifications, respectively) supports this conclusion, as demonstrated by the rigorous permutation testing to validate the significance of the cross-species overlap. Second, we identified several genes already known or implicated in T-ALL biology, such as Crebbp, Ikaros, and Abl, present within these identified syntenic MCRs. Together, these data support the relevance of this engineered murine model to a related uman cancer and its usefulness.

### Example 5

### Frequent Fbxw7 Inactivation in T-ALL

In this example, We identified Fbxw7 gene as a target of frequent inactivation or deletion in the TKO mouse model.

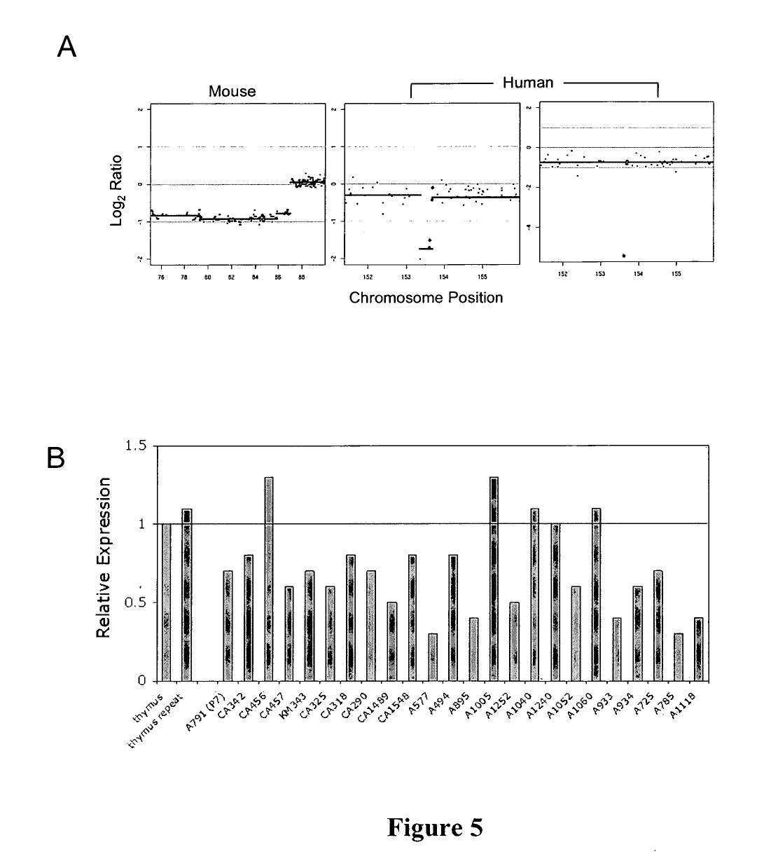

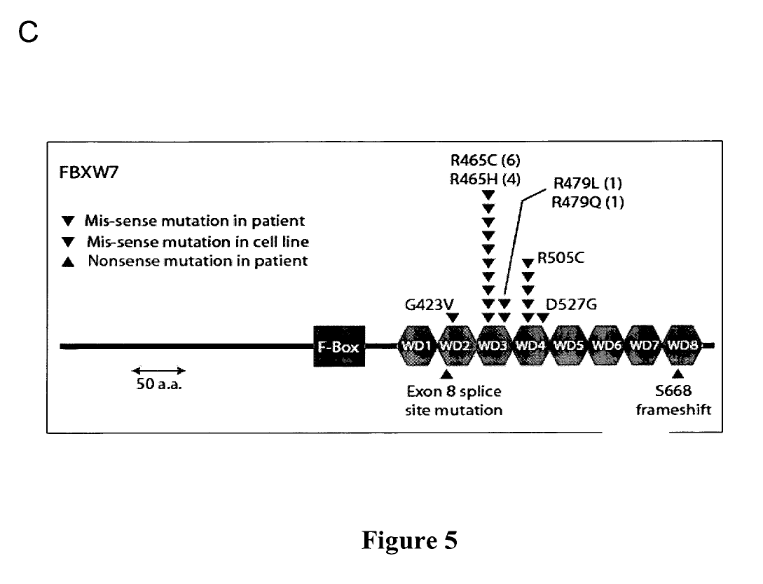

We observed that a few TKO tumors with minimal Notch1 expression exhibited elevated Notch4 or Jagged1 (Notch ligand) mRNA levels (data not shown). To investigate this observation, we conducted a more detailed examination of the genomic and expression status of known components in the Notch pathway The four core elements of the Notch signaling system include the Notch receptor, DSL (Delta, Serrate, Lag-2) ligands, CSL (CBF1, Suppressor of hairless, Lag-1) transcriptional cofactors, and target genes. Upon binding ligand the Notch signaling converts CSL from a transcriptional repressor to a transcriptional activator. TKO sample A577 was one of the two tumors harboring a syntenic MCR encompassing the Fbxw7 gene (MCR #18, Table 1). In human T-ALL, focal FBXW7 deletions including one case with a single-probe event were detected (FIG. 5A, right panel). Although extremely focal, the syntenic overlap across species made it unlikely that such deletion events represented copy number polymorphism. Indeed, FBXW7 re-sequencing in a cohort of human T-ALL clinical specimens (n=38) and cell lines (n=23) (Tables 4A, 4C, 6) revealed that FBXW7 was mutated or deleted in 11/23 of the human cell lines (48%) and 11/38 of the clinical samples (29%), marking this gene as one of those most commonly mutated in human T-ALL (Table 2). Consistent with reduced expression of Fbxw7 relative to non-neoplastic thymus in 19 of the 24 TKO lymphomas (FIG. 5B), these FBXW7 mutations in human T-ALL were predominantly mis-sense mutations, and particularly clustered in evolutionarily conserved residues of the third and fourth WD40 domains of the protein (FIG. 5C). Furthermore, re-sequencing of FBXW7 in matched normal bone marrows from several patients in complete remission showed that the two most frequently mutated positions (R465, R479) were acquired somatically (data not shown); along the same line, none of the identified mutations were found in public SNP databases, attesting to the likelihood that these mutations were somatic in nature. Finally, 19 of the 21 mutations were heterozygous, consistent with previous reports that Fbxw7 may act as a haplo-insufficient tumour suppressor gene.

FBXW7 is a key component of the E3 ubiquitin ligase responsible for binding the PEST domain of intracellular NOTCH1, leading to ubiquitination and degradation by the proteasome (N. Gupta-Rossi, et al., J Biol Chem 276 (37), 34371 (2001); C. Oberg, et al., J Biol Chem 276 (38), 35847 (2001); G. Wu, et al., Mol Cell Biol 21 (21), 7403 (2001)). PEST domain mutations in human T-ALL are thought to prolong the half-life of intracellular NOTCH1, raising the possibility that loss of FBXW7 function may cause similar effects on this pathway. To address this, we additionally characterized the human cell lines and clinical samples for NOTCH1 mutations (Table 2; Tables 4A, 4C, 6). Interestingly, there was no association between known functional mutations of NOTCH1 (HD-N, HD-C and PEST domains) and FBXW7 mutations (p=0.16). However, among samples with NOTCH1 mutations, FBXW7 mutations were found less frequently in samples with a mutated PEST domain (4/19; 21%) than samples with mutations of only the HD-N or HD-C domain (13/20; 65%; p=0.009 by Fisher exact test). One explanation of this observation is that mutations of FBXW7 and the PEST domain of NOTCH1 target the same degradation pathway, and little selective advantage accrues to the majority of leukaemias from mutating both components. At the same time, the lack of NOTCH1 and FBXW7 mutual exclusivity may suggest non-overlapping activities by FBXW7 on pathways other than NOTCH signaling.

### Example 6

### Pten Inactivation is a Common Event in Mouse and Human T-Cell Malignancy

In this example, We identified Pten gene as a target of frequent inactivation or deletion in the TKO mouse model.

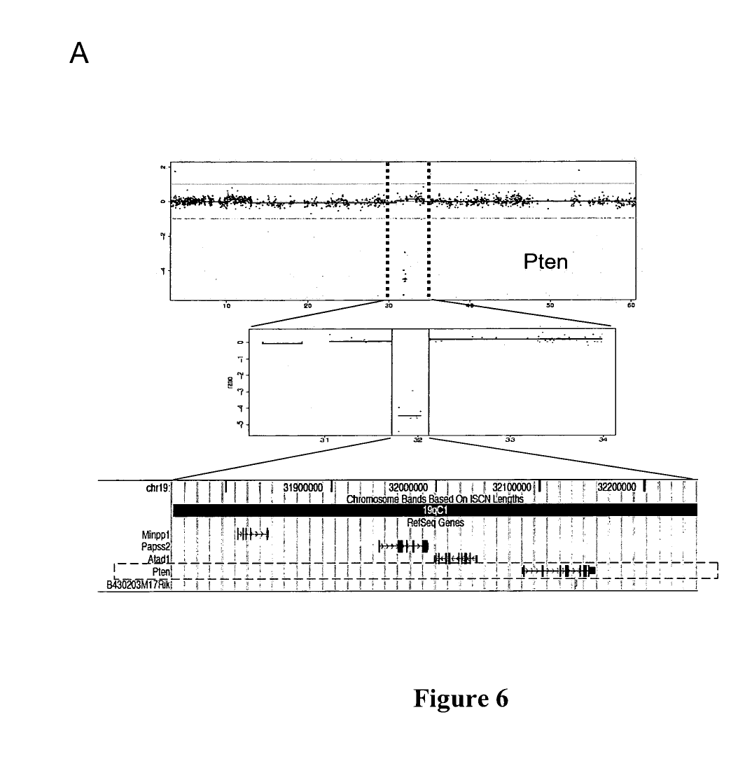

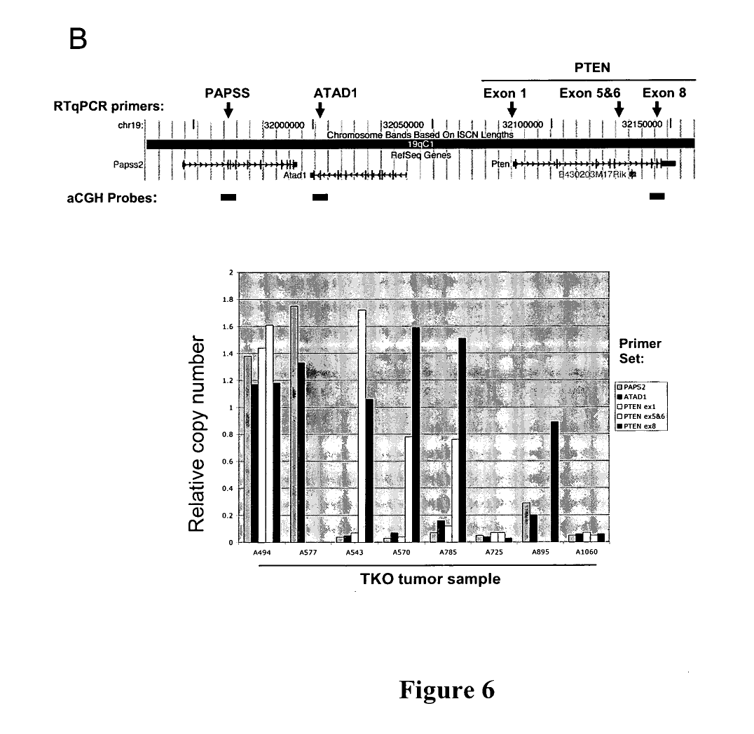

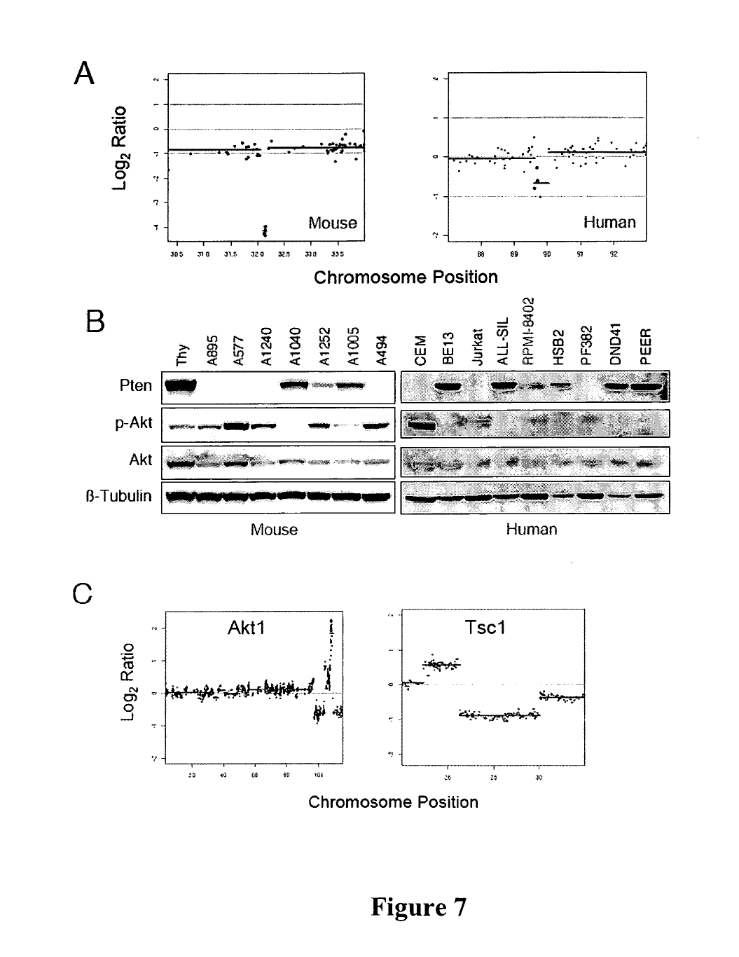

Focal deletion on chromosome 19, centering on the Pten gene, was among the most common genomic event in TKO lymphomas (Table 1, FIG. 2F). Using array-CGH, coupled with real-time PCR verification, we documented homozygous deletions of Pten in 15/35 (43%) TKO lymphomas (FIG. 6, FIG. 7A). PTEN is a well-known tumor suppressor and its inactivation in the murine thymus is known to generate T cell tumors (A. Suzuki, et al., Curr Biol 8 (21), 1169 (1998)). Correspondingly, array-CGH confirmed that 4 of the 26 human T-ALL samples (2 cell lines and 2 primary tumors) had sustained PTEN locus rearrangements. Additionally, re-sequencing of the 61 T-ALL cell lines and clinical specimens (Table 4) uncovered inactivating PTEN mutations in 9 cases (none of which were found in public SNP databases), but with no clear correlation with status of NOTCH1 mutations (Table 2, Table 6). In addition, we observed that PTEN mutations occurred more frequently in cell lines (7/23; 30.4%) than in clinical specimens (2/38; 5.2%) (Table 6). As these clinical specimens were derived from newly diagnosed cases whilst the cell lines were established primarily from relapses, without being bound by a particular theory, this difference in mutation frequency may suggest that PTEN inactivation is a later event associated with progression, among other possibilities.

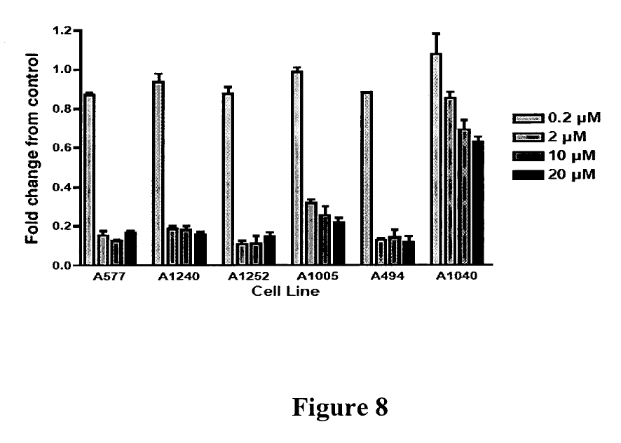

In addition to these genomic and genetic alterations, Northern and Western blot analyses and transcriptome profiling of the TKO and human T-ALL samples revealed a broader collection of tumors with low to undetectable PTEN expression (FIG. 7B, data not shown) with elevated phosphor-AKT. In addition to low PTEN expression, there appears to be additional mechanisms driving AKT activation as evidenced by the presence of focal Akt1 amplification and Tsc1 loss in two TKO samples (FIG. 7C; data not shown). Lastly, the biological significance of Pten status in TKO lymphoma is supported by their sensitivity to Akt inhibition in a Pten dependent manner (FIG. 8) in response to triciribine, a drug known to block Akt phosphorylation and shown to inhibit cells dependent on the Akt pathway. Briefly, twenty thousand cells were plated in triplicate in 96-well format and were incubated in standard media with varying doses of triciribine (BioMol, Plymouth Meeting, Pa.) or an equivalent concentration of vehicle (DMSO; Sigma Chemical, St. Louis, Mo.) for 2 days at 37° C., 5% CO2. At the end of the incubation period, cell growth was quantified with MTS assay (AqueousOne Cell Titer System; Promega, Madison, Wis.) and absorbance read at OD490. Relative cell growth was plotted against growth of the cell line in the equivalent amount DMSO alone. Experiments were repeated 3-5 times for each cell line and dose. As shown in FIG. 8, TKO cells with Pten mutations or deletions were sensitive to tricibine.

### Example 7

### Broad Comparison of TKO Genome with Diverse Human Cancers

In examples 3-6, Applicant identified and characterized Fbxw7 and Pten using the TKO mouse model. Both Fbxw7 and Pten have been previously identified as tumor suppressor genes. Thus their identification as mutated in human T-ALL provided proof of principle for the Applicants' approach and demonstrated that the mouse model described herein provides a powerful tool to cancer gene discovery. In this example, Applicants extended the cross-species genomic analyses to other human cancers.

While above cross-species comparison showed numerous concordant lesions in cancers of T cell origin, the fact that this instability model is driven by mechanisms of fundamental relevance (e.g., telomere dysfunction and p53 mutation) to many cancer types, including non-hematopoietic malignancies, suggested potentially broader relevance to other human cancers. A case in point is the Pten example above, in that PTEN is a bona fide tumor suppressor for multiple cancer types^{49,50}. To assess this, we extended the cross-species comparative genomic analyses to 6 other human cancer types (n=421) of hematopoietic, mesenchymal and epithelial origins, including multiple myeloma (n=67)⁵³, glioblastoma (n=38) (unpublished) and melanoma (n=123) (unpublished), as well as adenocarcinomas of the pancreas (n=30) (unpublished), lung (n=63)⁵⁴ and colon (n=74) (unpublished).

Compared against similarly defined MCR lists (i.e. MCR width<=10 Mb; see Example 4 and FIG. 5A) of each of these cancer types, Applicants found that 102 (61 amplifications and 41 deletions) of the 160 MCRs (64%) in the TKO genomes matched with at least one MCR in one human array-CGH dataset (FIG. 5A), with strong statistical significance attesting to non-randomness of this degree of overlap. Confidence in the genetic relevance of these syntenic events was further bolstered by the observation that more than half of these syntenic MCRs (38 of 61 amplifications or 62%; 22 of 41 deletions or 53%) overlapped with MCRs recurrent in two or more human tumor types (FIG. 5B). Moreover, a significant proportion of the TKO MCRs are evolutionarily conserved in human tumors of non-hematopoietic origin (FIG. 5C). Among the 61 amplifications with syntenic hits, 58 of them (95%) were observed in solid tumors, while the remaining 3 were uniquely found in myeloma (FIG. 5C). Similarly, 33 of the 41 (80%) syntenic deletions were present in solid tumors (FIG. 5C). In particular, Applicants found that p53 was present in a deletion MCR in 5 of 7 human cancer types, while Myc was the target of an amplification that overlapped with 6 human cancers. This substantial overlap with diverse human cancers was unexpected.

Next, Applicants determined whether these syntenic MCRs targeted known cancer genes to provide an additional level of validation for these TKO genomic events. Among the 363 genes listed on the Cancer Gene Census⁵⁵, 237 genes have a mouse homolog based on NCBI homologene (see Example 4). Of these, 24 known cancer genes were found to be resident within one of the 104 syntenic MCRs (Table 7). These included 17 oncogenes in amplifications and 7 tumor suppressor genes in deletions. The majority of these syntenic MCRs do not contain known cancer genes, raising the strong possibility that re-sequencing focused on resident genes of syntenic MCRs may provide a high-yield strategy to identify somatic mutations in human cancers, a thesis supported by the FBXW7 and PTEN examples.

The practice of the various aspects of the present invention may employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Current Protocols in Molecular Biology, by Ausubel et al., Greene Publishing Associates (1992, and Supplements to 2003); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986); Coffin et al., Retroviruses, Cold Spring Harbor Laboratory Press; Cold Spring Harbor, N.Y. (1997); Bast et al., Cancer Medicine, 5th ed., Frei, Emil, editors, BC Decker Inc., Hamilton, Canada (2000); Lodish et al., Molecular Cell Biology, 4th ed., W. H. Freeman & Co., New York (2000); Griffiths et al., Introduction to Genetic Analysis, 7th ed., W. H. Freeman & Co., New York (1999); Gilbert et al., Developmental Biology, 6th ed., Sinauer Associates, Inc., Sunderland, Mass. (2000); and Cooper, The Cell—A Molecular Approach, 2nd ed., Sinauer Associates, Inc., Sunderland, Mass. (2000). All patents, patent applications and references cited herein are incorporated in their entirety by reference.

### REFERENCES

R. C. O'Hagan, C. W. Brennan, A. Strahs et al., Cancer Res 63 (17), 5352 (2003).
N. Bardeesy, A. J. Aguirre, G. C. Chu et al., Proc Natl Acad Sci USA 103 (15), 5947 (2006).
M. Kim, J. D. Gans, C. Nogueira et al., Cell 125 (7), 1269 (2006).
L. Zender, M. S. Spector, W. Xue et al., Cell 125 (7), 1253 (2006).
A. Sweet-Cordero, G. C. Tseng, H. You et al., Genes Chromosomes Cancer 45 (4), 338 (200S. E. Artandi, S. Chang, S. L. Lee et al., Nature 406 (6796), 641 (2000).
C. Zhu, K. D. Mills, D. O. Ferguson et al., Cell 109 (7), 811 (2002).
G. A. Lang, T. Iwakuma, Y. A. Suh et al., Cell 119 (6), 861 (2004).
K. P. Olive, D. A. Tuveson, Z. C. Ruhe et al., Cell 119 (6), 847 (2004).
S. R. Hingorani, L. Wang, A. S. Multani et al., Cancer Cell 7 (5), 469 (2005).
A. P. Weng, A. A. Ferrando, W. Lee et al., Science 306 (5694), 269 (2004).
F. Radtke, A. Wilson, S. J. Mancini et al., Nat Immunol 5 (3), 247 (2004).
L. W. Ellisen, J. Bird, D.C. West et al., Cell 66 (4), 649 (1991).
J. H. Mao, J. Perez-Losada, D. Wu et al., Nature 432 (7018), 775 (2004).
N. Gupta-Rossi, O. Le Bail, H. Gonen et al., J Biol Chem 276 (37), 34371 (2001).
C. Oberg, J. Li, A. Pauley et al., J Biol Chem 276 (38), 35847 (2001).
G. Wu, S. Lyapina, I. Das et al., Mol Cell Biol 21 (21), 7403 (2001).
A. Suzuki, J. L. de la Pompa, V. Stambolic et al., Curr Biol 8 (21), 1169 (1998).
L. Yang, H. C. Dan, M. Sun et al., Cancer Res 64 (13), 4394 (2004).
D. R. Carrasco, G. Tonon, Y. Huang et al., Cancer Cell 9 (4), 313 (2006).
A. B. Olshen, E. S. Venkatraman, R. Lucito et al., Biostatistics 5 (4), 557 (2004).
A. J. Aguirre, C. Brennan, G. Bailey et al., Proc Natl Acad Sci USA 101 (24), 9067 (2004).
M. R. Mansour, D. C. Linch, L. Foroni et al., Leukemia 20 (3), 537 (2006).
H. Davies, C. Hunter, R. Smith et al., Cancer Res 65 (17), 7591 (2005).
Wong, G. T. et. al, J. Biol. Chem., Vol. 279, Issue 13, 12876-12882, Mar. 26, 2004

### SEQUENCES

Mm Dvl1 cDNA (Homo sapiens)

| SEQ ID NO: 1 | |
|---|---|
| 1 | atggcggaga ccaagattat ctaccacatg gacgaggagg agacgccgta |
| | cctggtcaag |
| 61 | ctgcccgtgg cccccgagcg cgtcacgctg gccgacttca agaacgtgct |
| | cagcaaccgg |
| 121 | cccgtgcacg cctacaaatt cttctttaag tccatggacc aggacttcgg |
| | ggtggtgaag |
| 181 | gaggagatct ttgatgacaa tgccaagctt ccctgcttca acggccgcgt |
| | ggtctcctgg |
| 241 | ctggtcctgg ctgagggtgc tcactcggat gcggggtccc agggcacgga |
| | cagccacaca |
| 301 | gacctgcccc cgcctcttga gcggacaggc ggcatcgggg actcccggcc |
| | cccctccttc |
| 361 | cacccaaatg tggccagcag ccgtgacggg atggacaacg agacaggcac |
| | ggagtccatg |
| 421 | gtcagtcacc ggcgggagcg tgcccgacgc cggaaccgcg aggaggccgc |
| | ccggaccaat |
| 481 | gggcacccaa ggggagaccg acggcgggat gtggggctgc ccccagacag |
| | cgcgtccacc |
| 541 | gccctcagca gcgagcttga gtccagcagc tttgtggact cggacgagga |
| | tggcagcacg |
| 601 | agcaggctca gcagctccac ggagcagagc acctcatcca gactcatccg |
| | gaagcacaaa |
| 661 | cgccggcgga ggaagcagcg ccttcggcag gcggaccggg cctcctcctt |
| | cagcagcata |
| 721 | accgactcca ccatgtccct caacatcgtc actgtcacgc tcaacatgga |
| | aagacatcac |
| 781 | tttctgggca tcagcatcgt ggggcagagc aacgaccgtg gagacggcgg |
| | catctacatt |
| 841 | ggctccatca tgaagggcgg ggctgtggcc gctgacggcc gcatcgagcc |
| | cggcgacatg |
| 901 | ttgctgcagg tgaatgacgt gaactttgag aacatgagca atgacgatgc |
| | cgtgcgggtg |
| 961 | ctgcgggaga tcgtttccca gacggggccc atcagcctca ctgtggccaa |
| | gtgctgggac |
| 1021 | ccaacgcccc gaagctactt caccgtccca cgggctgacc cggtgcggcc |
| | catcgacccc |
| 1081 | gccgcctggc tgtcccacac ggcggcactg acaggagccc tgccccgcta |
| | cgagctggaa |
| 1141 | gaggcgccgc tgacggtgaa gagtgacatg agcgccgtcg tccgggtcat |
| | gcagctgcca |
| 1201 | gactcgggac tggagatccg cgaccgcatg tggctcaaga tcaccatcgc |
| | caatgccgtc |
| 1261 | atcggggcgg acgtggtgga ctggctgtac acacacgtgg agggcttcaa |
| | ggagcggcgg |
| 1321 | gaggcccgga agtacgccag cagcttgctg aagcacggct tcctgcggca |
| | cacggtcaac |
| 1381 | aagatcacct tctccgagca gtgctactac gtcttcgggg atctctgcag |
| | caatctcgcc |
| 1441 | accctgaacc tcaacagtgg ctccagtggg acttcggatc aggacacgct |
| | ggccccgctg |
| 1501 | ccccacccgg ctgccccctg gcctctgggt cagggctacc cctaccagta |
| | cccgggaccc |
| 1561 | ccaccctgct tcccgcctgc ctaccaggac ccgggcttta gctatggcag |
| | cggcagcacc |
| 1621 | gggagtcagc agagtgaagg gagcaaaagc agtgggtcca cccggagcag |
| | ccgccgggcc |
| 1681 | ccgggccgtg agaaggagcg tcgggcggcg ggagctgggg gcagtggcag |
| | tgaatcggat |
| 1741 | cacacggcac cgagtggggt ggggagcagc tggcgagagc gtccggccgg |
| | ccagctcagc |
| 1801 | cgtggcagca gcccacgcag tcaggcctcg gctaccgccc cggggctccc |
| | cccgccccac |
| 1861 | cccacgacca aggcctatac agtggtgggg gggccacccg ggggaccccc |
| | tgtccgggag |
| 1921 | ctggctgccg tccccccgga attgacaggc agccgccagt ccttccagaa |
| | ggctatgggg |
| 1981 | aacccctgcg agttcttcgt ggacatcatg tga |

Mm DVL1 protein (Homo sapiens)

SEQ ID NO: 2

| | |
|---|---|
| 1 | maetkiiyhm deeetpylvk lpvapervtl adfknvlsnr |
| | pvhaykfffk smdqdfgvvk |
| 61 | eeifddnakl pcfngrvvsw lvlaegahsd agsqgtdsht |
| | dlppplertg gigdsrppsf |
| 121 | hpnvassrdg mdnetgtesm vshrrerarr rnreeaartn |
| | ghprgdrrrd vglppdsast |
| 181 | alsselesss fvdsdedgst srlsssteqs tssrlirkhk |
| | rrrrkqrlrq adrassfssi |
| 241 | tdstmslniv tvtlnmerhh flgisivgqs ndrgdggiyi |
| | gsimkggava adgriepgdm |
| 301 | llqvndvnfe nmsnddavrv lreivsqtgp isltvakcwd |
| | ptprsyftvp radpvrpidp |
| 361 | aawlshtaal tgalpryele eapltvksdm savvrvmqlp |
| | dsgleirdrm wlkitianav |
| 421 | igadvvdwly thvegfkerr earkyassll khgflrhtvn |
| | kitfseqcyy vfgdlcsnla |
| 481 | tlnlnsgssg tsdqdtlapl phpaapwplg qgypyqypgp |
| | ppcfppayqd pgfsygsgst |
| 541 | gsqqsegsks sgstrssrra pgrekerraa gaggsgsesd |
| | htapsgvgss wrerpagqls |
| 601 | rgssprsqas atapglppph pttkaytvvg gppggppvre |
| | laavppeltg srqsfqkamg |
| 661 | npceffvdim |

Ccnl2 cDNA (Homo sapiens)

| SEQ ID NO: 3 | |
|---|---|
| 1 | atggcggcgg cggcggcggc ggctggtgct gcagggtcgg cagctcccgc |
| | ggcagcggcc |
| 61 | ggcgccccgg gatctggggg cgcaccctca gggtcgcagg gggtgctgat |
| | cggggacagg |
| 121 | ctgtactccg gggtgctcat caccttggag aactgcctcc tgcctgacga |
| | caagctccgt |
| 181 | ttcacgccgt ccatgtcgag cggcctcgac accgacacag agaccgacct |
| | ccgcgtggtg |
| 241 | ggctgcgagc tcatccaggc ggccggtatc ctgctccgcc tgccgcaggt |
| | ggccatggct |
| 301 | accgggcagg tgttgttcca gcggttcttt tataccaagt ccttcgtgaa |
| | gcactccatg |
| 361 | gagcatgtgt caatggcctg tgtccacctg gcttccaaga tagaagaggc |
| | cccaagacgc |
| 421 | atacgggacg tcatcaatgt gtttcaccgc cttcgacagc tgagagacaa |
| | aaagaagccc |
| 481 | gtgcctctac tactggatca agattatgtt aatttaaaga accaaattat |
| | aaaggcggaa |
| 541 | agacgagttc tcaaagagtt gggtttctgc gtccatgtga agcatcctca |
| | taagataatc |
| 601 | gttatgtacc ttcaggtgtt agagtgtgag cgtaaccaac acctggtcca |
| | gacctcatgg |
| 661 | aattacatga acgacagcct tcgcaccgac gtcttcgtgc ggttccagcc |
| | agagagcatc |
| 721 | gcctgtgcct gcatttatct tgctgcccgg acgctggaga tccctttgcc |
| | caatcgtccc |
| 781 | cattggtttc ttttgtttgg agcaactgaa gaagaaattc aggaaatctg |
| | cttaaagatc |
| 841 | ttgcagcttt atgctcggaa aaaggttgat ctcacacacc tggagggtga |
| | agtggaaaaa |
| 901 | agaaagcacg ctatcgaaga ggcaaaggcc caagcccggg gcctgttgcc |
| | tgggggcaca |
| 961 | caggtgctgg atggtacctc ggggttctct cctgccccca agctggtgga |
| | atcccccaaa |
| 1021 | gaaggtaaag ggagcaagcc ttccccactg tctgtgaaga acaccaagag |
| | gaggctggag |
| 1081 | ggcgccaaga aagccaaggc ggacagcccc gtgaacggct tgccaaaggg |
| | gcgagagagt |
| 1141 | cggagtcgga gccggagccg tgagcagagc tactcgaggt ccccatcccg |
| | atcagcgtct |
| 1201 | cctaagagga ggaaaagtga cagcggctcc acatctggtg ggtccaagtc |
| | gcagagccgc |
| 1261 | tcccggagca ggagtgactc cccaccgaga caggcccccc gcagcgctcc |
| | ctacaaaggc |
| 1321 | tctgagattc ggggctcccg gaagtccaag gactgcaagt acccccagaa |
| | gccacacaag |
| 1381 | tctcggagcc ggagttcttc ccgttctcga agcaggtcac gggagcgggc |
| | ggataatccg |
| 1441 | ggaaaataca agaagaaaag tcattactac agagatcagc gacgagagcg |
| | ctcgaggtcg |
| 1501 | tatgaacgca caggccgtcg ctatgagcgg gaccaccctg ggcacagcag |
| | gcatcggagg |
| 1561 | tga |

CCNL2 protein (Homo sapiens)

SEQ ID NO: 4

| | |
|---|---|
| 1 | maaaaaaaga agsaapaaaa gapgsggaps gsqgvligdr |
| | lysgvlitle ncllpddklr |
| 61 | ftpsmssgld tdtetdlrvv gceliqaagi llrlpqvama |
| | tgqvlfqrff ytksfvkhsm |
| 121 | ehvsmacvhl askieeaprr irdvinvfhr lrqlrdkkkp |
| | vpllldqdyv nlknqiikae |
| 181 | rrvlkelgfc vhvkhphkii vmylqvlece rnqhlvqtsw |
| | nymndslrtd vfvrfqpesi |
| 241 | acaciylaar tleiplpnrp hwfllfgate eeiqeiclki |
| | lqlyarkkvd lthlegevek |
| 301 | rkhaieeaka qargllpggt qvldgtsgfs papklvespk |
| | egkgskpspl svkntkrrle |
| 361 | gakkakadsp vnglpkgres rsrsrsreqs ysrspsrsas |
| | pkrrksdsgs tsggsksqsr |
| 421 | srsrsdsppr qaprsapykg seirgsrksk dckypqkphk |
| | srsrsssrsr srsreradnp |
| 481 | gkykkkshyy rdqrrersrs yertgrryer dhpghsrhrr |

Aurkaip1 cDNA (Homo sapiens)

SEQ ID NO: 5

| | |
|---|---|
| 1 | atgctcctgg ggcgcctgac ttcccagctg ttgagggccg |
| | ttccttgggc aggcggccgc |
| 61 | ccgccttggc ccgtctctgg agtgctgggc agccgggtct |
| | gcgggcccct ttacagcaca |
| 121 | tcgccggccg gcccaggtag ggcggcctct ctccctcgca |
| | agggggccca gctggagctg |
| 181 | gaggagatgc tggtccccag gaagatgtcc gtcagccccc |
| | tggagagctg gctcacggcc |
| 241 | cgctgcttcc tgcccagact ggataccggg accgcaggga |
| | ctgtggctcc accgcaatcc |
| 301 | taccagtgtc cgcccagcca gataggggaa ggggccgagc |
| | agggggatga aggcgtcgcg |
| 361 | gatgcgcctc aaattcagtg caaaaacgtg ctgaagatcc |
| | gccggcggaa gatgaaccac |
| 421 | cacaagtacc ggaagctggt gaagaagacg cggttcctgc |
| | ggaggaaggt ccaggaggga |
| 481 | cgcctgagac gcaagcagat caagttcgag aaagacctga |
| | ggcgcatctg gctgaaggcg |
| 541 | gggctaaagg aagcccccga aggctggcag acccccaaga |
| | tctacctgcg gggcaaatga |

AURKAIP1 Protein (Homo sapiens)

SEQ ID NO: 6

| | |
|---|---|
| 1 | mllgrltsql lravpwaggr ppwpvsgvlg srvcgplyst |
| | spagpgraas lprkgaqlel |
| 61 | eemlvprkms vspleswlta rcflprldtg tagtvappqs |
| | yqcppsqige gaeqgdegva |
| 121 | dapqiqcknv lkirrrkmnh hkyrklvkkt rflrrkvqeg |
| | rlrrkqikfe kdlrriwlka |
| 181 | glkeapegwq tpkiylrgk |

Myb cDNA (Homo sapiens)

| SEQ ID NO: 7 | |
|---|---|
| 1 | atggcccgaa gaccccggca cagcatatat agcagtgacg aggatgatga |
| | ggactttgag |
| 61 | atgtgtgacc atgactatga tgggctgctt cccaagtctg gaaagcgtca |
| | cttggggaaa |
| 121 | acaaggtgga cccgggaaga ggatgaaaaa ctgaagaagc tggtggaaca |
| | gaatggaaca |
| 181 | gatgactgga aagttattgc caattatctc ccgaatcgaa cagatgtgca |
| | gtgccagcac |
| 241 | cgatggcaga aagtactaaa ccctgagctc atcaagggtc cttggaccaa |
| | agaagaagat |
| 301 | cagagagtga tagagcttgt acagaaatac ggtccgaaac gttggtctgt |
| | tattgccaag |
| 361 | cacttaaagg ggagaattgg aaaacaatgt agggagaggt ggcataacca |
| | cttgaatcca |
| 421 | gaagttaaga aaacctcctg gacagaagag gaagacagaa ttatttacca |
| | ggcacacaag |
| 481 | agactgggga acagatgggc agaaatcgca aagctactgc ctggacgaac |
| | tgataatgct |
| 541 | atcaagaacc actggaattc tacaatgcgt cggaaggtcg aacaggaagg |
| | ttatctgcag |
| 601 | gagtcttcaa aagccagcca gccagcagtg gccacaagct tccagaagaa |
| | cagtcatttg |
| 661 | atgggttttg ctcaggctcc gcctacagct caactccctg ccactggcca |
| | gcccactgtt |
| 721 | aacaacgact attcctatta ccacatttct gaagcacaaa atgtctccag |
| | tcatgttcca |
| 781 | taccctgtag cgttacatgt aaatatagtc aatgtccctc agccagctgc |
| | cgcagccatt |
| 841 | cagagacact ataatgatga agaccctgag aaggaaaagc gaataaagga |
| | attagaattg |
| 901 | ctcctaatgt caaccgagaa tgagctaaaa ggacagcagg tgctaccaac |
| | acagaaccac |
| 961 | acatgcagct accccgggtg gcacagcacc accattgccg accacaccag |
| | acctcatgga |
| 1021 | gacagtgcac ctgtttcctg tttgggagaa caccactcca ctccatctct |
| | gccagcggat |
| 1081 | cctggctccc tacctgaaga aagcgcctcg ccagcaaggt gcatgatcgt |
| | ccaccagggc |
| 1141 | accattctgg ataatgttaa gaacctctta gaatttgcag aaacactcca |
| | atttatagat |
| 1201 | tctttcttaa acacttccag taaccatgaa aactcagact tggaaatgcc |
| | ttctttaact |
| 1261 | tccacccccc tcattggtca caaattgact gttacaacac catttcatag |
| | agaccagact |
| 1321 | gtgaaaactc aaaaggaaaa tactgttttt agaaccccag ctatcaaaag |
| | gtcaatctta |
| 1381 | gaaagctctc caagaactcc tacaccattc aaacatgcac ttgcagctca |
| | agaaattaaa |
| 1441 | tacggtcccc tgaagatgct acctcagaca ccctctcatc tagtagaaga |
| | tctgcaggat |
| 1501 | gtgatcaaac aggaatctga tgaatctgga attgttgctg agtttcaaga |
| | aaatggacca |
| 1561 | cccttactga agaaaatcaa acaagaggtg gaatctccaa ctgataaatc |
| | aggaaacttc |
| 1621 | ttctgctcac accactggga aggggacagt ctgaataccc aactgttcac |
| | gcagacctcg |
| 1681 | cctgtggcag atgcaccgaa tattcttaca agctccgttt taatggcacc |
| | agcatcagaa |
| 1741 | gatgaagaca atgttctcaa agcatttaca gtacctaaaa acaggtccct |
| | ggcgagcccc |
| 1801 | ttgcagcctt gtagcagtac ctgggaacct gcatcctgtg gaaagatgga |
| | ggagcagatg |
| 1861 | acatcttcca gtcaagctcg taaatacgtg aatgcattct cagcccggac |
| | gctggtcatg |
| 1921 | tga |

MYB Protein (Homo sapiens)

SEQ ID NO: 8

| | |
|---|---|
| 1 | marrprhsiy ssdeddedfe mcdhdydgll pksgkrhlgk |
| | trwtreedek lkklveqngt |
| 61 | ddwkvianyl pnrtdvqcqh rwqkvlnpel ikgpwtkeed |
| | qrvielvqky gpkrwsviak |
| 121 | hlkgrigkqc rerwhnhlnp evkktswtee edriiyqahk |
| | rlgnrwaeia kllpgrtdna |
| 181 | iknhwnstmr rkveqegylq esskasqpav atsfqknshl |
| | mgfaqappta qlpatgqptv |
| 241 | nndysyyhis eaqnvsshvp ypvalhvniv nvpqpaaaai |
| | qrhyndedpe kekrikelel |
| 301 | llmstenelk gqqvlptqnh tcsypgwhst tiadhtrphg |
| | dsapvsclge hhstpslpad |
| 361 | pgslpeesas parcmivhqg tildnvknll efaetlqfid |
| | sflntssnhe nsdlempslt |
| 421 | stplighklt vttpfhrdqt vktqkentvf rtpaikrsil |
| | essprtptpf khalaaqeik |
| 481 | ygplkmlpqt pshlvedlqd vikqesdesg ivaefqengp |
| | pllkkikqev esptdksgnf |
| 541 | fcshhwegds lntqlftqts pvadapnilt ssvlmapase |
| | dednvlkaft vpknrslasp |
| 601 | lqpcsstwep ascgkmeeqm tsssqarkyv nafsartlvm |

Ahi1 cDNA (Homo sapiens)

| SEQ ID NO: 9 | |
|---|---|
| 1 | atgcctacag ctgagagtga agcaaaagta aaaaccaaag ttcgctttga |
| | agaattgctt |
| 61 | aagacccaca gtgatctaat gcgtgaaaag aaaaaactga agaaaaaact |
| | tgtcaggtct |
| 121 | gaagaaaaca tctcacctga cactattaga agcaatcttc actatatgaa |
| | agaaactaca |
| 181 | agtgatgatc ccgacactat tagaagcaat cttccccata ttaaagaaac |
| | tacaagtgat |
| 241 | gatgtaagtg ctgctaacac taacaacctg aagaagagca cgagagtcac |
| | taaaaacaaa |
| 301 | ttgaggaaca cacagttagc aactgaaaat cctaatggtg atgctagtgt |
| | agaggaagac |
| 361 | aaacaaggaa agccaaataa aaaggtgata aagacggtgc cccagttgac |
| | tacacaagac |
| 421 | ctgaaaccgg aaactcctga gaataaggtt gattctacac accagaaaac |
| | acatacaaag |
| 481 | ccacagccag gcgttgatca tcagaaaagt gagaaggcaa atgagggaag |
| | agaagagact |
| 541 | gatttagaag aggatgaaga attgatgcaa gcatatcagt gccatgtaac |
| | tgaagaaatg |
| 601 | gcaaaggaga ttaagaggaa aataagaaag aaactgaaag aacagttgac |
| | ttactttccc |
| 661 | tcagatactt tattccatga tgacaaacta agcagtgaaa aaaggaaaaa |
| | gaaaaaggaa |
| 721 | gttccagtct tctctaaagc tgaaacaagt acattgacca tctctggtga |
| | cacagttgaa |
| 781 | ggtgaacaaa agaaagaatc ttcagttaga tcagtttctt cagattctca |
| | tcaagatgat |
| 841 | gaaataagct caatggaaca aagcacagaa gacagcatgc aagatgatac |
| | aaaacctaaa |
| 901 | ccaaaaaaaa caaaaaagaa gactaaagca gttgcagata ataatgaaga |
| | tgttgatggt |
| 961 | gatggtgttc atgaaataac aagccgagat agcccggttt atcccaaatg |
| | tttgcttgat |
| 1021 | gatgaccttg tcttgggagt ttacattcac cgaactgata gacttaagtc |
| | agattttatg |
| 1081 | atttctcacc caatggtaaa aattcatgtg gttgatgagc atactggtca |
| | atatgtcaag |
| 1141 | aaagatgata gtggacggcc tgtttcatct tactatgaaa aagagaatgt |
| | ggattatatt |
| 1201 | cttcctatta tgacccagcc atatgatttt aaacagttaa aatcaagact |
| | tccagagtgg |
| 1261 | gaagaacaaa ttgtatttaa tgaaaatttt ccctatttgc ttcgaggctc |
| | tgatgagagt |
| 1321 | cctaaagtca tcctgttctt tgagattctt gatttcttaa gcgtggatga |
| | aattaagaat |
| 1381 | aattctgagg ttcaaaacca agaatgtggc tttcggaaaa ttgcctgggc |
| | atttcttaag |
| 1441 | cttctgggag ccaatggaaa tgcaaacatc aactcaaaac ttcgcttgca |
| | gctatattac |
| 1501 | ccacctacta agcctcgatc cccattaagt gttgttgagg catttgaatg |
| | gtggtcaaaa |
| 1561 | tgtccaagaa atcattaccc atcaacactg tacgtaactg taagaggact |
| | gaaagttcca |
| 1621 | gactgtataa agccatctta ccgctctatg atggctcttc aggaggaaaa |
| | aggtaaacca |
| 1681 | gtgcattgtg aacgtcacca tgagtcaagc tcagtagaca cagaacctgg |
| | attagaagag |
| 1741 | tcaaaggaag taataaagtg gaaacgactc cctgggcagg cttgccgtat |
| | cccaaacaaa |
| 1801 | cacctcttct cactaaatgc aggagaacga ggatgttttt gtcttgattt |
| | ctcccacaat |
| 1861 | ggaagaatat tagcagcagc ttgtgccagc cgggatggat atccaattat |
| | tttatatgaa |
| 1921 | attccttctg gacgtttcat gagagaattg tgtggccacc tcaatatcat |
| | ttatgatctt |
| 1981 | tcctggtcaa aagatgatca ctacatcctt acttcatcat ctgatggcac |
| | tgccaggata |
| 2041 | tggaaaaatg aaataaacaa tacaaatact ttcagagttt tacctcatcc |
| | ttcttttgtt |
| 2101 | tacacggcta aattccatcc agctgtaaga gagctagtag ttacaggatg |
| | ctatgattcc |
| 2161 | atgatacgga tatggaaagt tgagatgaga gaagattctg ccatattggt |
| | ccgacagttt |
| 2221 | gatgttcaca aaagttttat caactcactt tgttttgata ctgaaggtca |
| | tcatatgtat |
| 2281 | tcaggagatt gtacaggggt gattgttgtt tggaatacct atgtcaagat |
| | taatgatttg |
| 2341 | gaacattcag tgcaccactg gactataaat aaggaaatta aagaaactga |
| | gtttaaggga |
| 2401 | attccaataa gttatttgga gattcatccc aatggaaaac gtttgttaat |
| | ccataccaaa |
| 2461 | gacagtactt tgagaattat ggatctccgg atattagtag caaggaagtt |
| | tgtaggagca |
| 2521 | gcaaattatc gggagaagat tcatagtact ttgactccat gtgggacttt |
| | tctgtttgct |
| 2581 | ggaagtgagg atggtatagt gtatgtttgg aacccagaaa caggagaaca |
| | agtagccatg |
| 2641 | tattctgact tgccattcaa gtcacccatt cgagacattt cttatcatcc |
| | atttgaaaat |
| 2701 | atggttgcat tctgtgcatt tgggcaaaat gagccaattc ttctgtatat |
| | ttacgatttc |
| 2761 | catgttgccc agcaggaggc tgaaatgttc aaacgctaca atggaacatt |
| | tccattacct |
| 2821 | ggaatacacc aaagtcaaga tgccctatgt acctgtccaa aactacccca |
| | tcaaggctct |
| 2881 | tttcagattg atgaatttgt ccacactgaa agttcttcaa cgaagatgca |
| | gctagtaaaa |
| 2941 | cagaggcttg aaactgtcac agaggtgata cgttcctgtg ctgcaaaagt |
| | caacaaaaat |
| 3001 | ctctcattta cttcaccacc agcagtttcc tcacaacagt ctaagttaaa |
| | gcagtcaaac |
| 3061 | atgctgaccg ctcaagagat tctacatcag tttggtttca ctcagaccgg |
| | gattatcagc |
| 3121 | atagaaagaa agccttgtaa ccatcaggta gatacagcac caacggtagt |
| | ggctctttat |
| 3181 | gactacacag cgaatcgatc agatgaacta accatccatc gcggagacat |
| | tatccgagtg |
| 3241 | tttttcaaag ataatgaaga ctggtggtat ggcagcatag gaaagggaca |
| | ggaaggttat |
| 3301 | tttccagcta atcatgtggc tagtgaaaca ctgtatcaag aactgcctcc |
| | tgagataaag |
| 3361 | gagcgatccc ctcctttaag ccctgaggaa aaaactaaaa tagaaaaatc |
| | tccagctcct |
| 3421 | caaaagcaat caatcaataa gaacaagtcc caggacttca gactaggctc |
| | agaatctatg |
| 3481 | acacattctg aaatgagaaa agaacagagc catgaggacc aaggacacat |
| | aatggataca |
| 3541 | cggatgagga agaacaagca agcaggcaga aaagtcactc taatagagta a |

AHl1 Protein (Homo sapiens)

SEQ ID NO: 10

| | |
|---|---|
| 1 | mptaeseakv ktkvrfeell kthsdlmrek kklkkklvrs |
| | eenispdtir snlhymkett |
| 61 | sddpdtirsn lphikettsd dvsaantnnl kkstrvtknk |
| | lrntqlaten pngdasveed |
| 121 | kqgkpnkkvi ktvpqlttqd lkpetpenkv dsthqkthtk |
| | pqpgvdhqks ekanegreet |
| 181 | dleedeelmq ayqchvteem akeikrkirk klkeqltyfp |
| | sdtlfhddkl ssekrkkkke |
| 241 | vpvfskaets tltisgdtve geqkkessvr svssdshqdd |
| | eissmeqste dsmqddtkpk |
| 301 | pkktkkktka vadnnedvdg dgvheitsrd spvypkclld |
| | ddlvlgvyih rtdrlksdfm |
| 361 | ishpmvkihv vdehtgqyvk kddsgrpvss yyekenvdyi |
| | lpimtqpydf kqlksrlpew |
| 421 | eeqivfnenf pyllrgsdes pkvilffeil dflsvdeikn |
| | nsevqnqecg frkiawaflk |
| 481 | llgangnani nsklrlqlyy pptkprspls vveafewwsk |
| | cprnhypstl yvtvrglkvp |
| 541 | dcikpsyrsm malqeekgkp vhcerhhess svdtepglee |
| | skevikwkrl pgqacripnk |
| 601 | hlfslnager gcfcldfshn grilaaacas rdgypiilye |
| | ipsgrfmrel cghlniiydl |
| 661 | swskddhyil tsssdgtari wkneinntnt frvlphpsfv |
| | ytakfhpavr elvvtgcyds |
| 721 | miriwkvemr edsailvrqf dvhksfinsl cfdteghhmy |
| | sgdctgvivv wntyvkindl |
| 781 | ehsvhhwtin keiketefkg ipisyleihp ngkrllihtk |
| | dstlrimdlr ilvarkfvga |
| 841 | anyrekihst ltpcgtflfa gsedgivyvw npetgeqvam |
| | ysdlpfkspi rdisyhpfen |
| 901 | mvafcafgqn epillyiydf hvaqqeaemf kryngtfplp |
| | gihqsqdalc tcpklphqgs |
| 961 | fqidefvhte ssstkmqlvk qrletvtevi rscaakvnkn |
| | lsftsppavs sqqsklkqsn |
| 1021 | mltaqeilhq fgftqtgiis ierkpcnhqv dtaptvvaly |
| | dytanrsdel tihrgdiirv |
| 1081 | ffkdnedwwy gsigkgqegy fpanhvaset lyqelppeik |
| | erspplspee ktkiekspap |
| 1141 | qkqsinknks qdfrlgsesm thsemrkeqs hedqghimdt |
| | rmrknkqagr kvtlie |

Runx1 cDNA (Homo sapiens)

SEQ ID NO: 11

| | |
|---|---|
| 1 | atggcttcag acagcatatt tgagtcattt ccttcgtacc |
| | cacagtgctt catgagagaa |
| 61 | tgcatacttg gaatgaatcc ttctagagac gtccacgatg |
| | ccagcacgag ccgccgcttc |
| 121 | acgccgcctt ccaccgcgct gagcccaggc aagatgagcg |
| | aggcgttgcc gctgggcgcc |
| 181 | ccggacgccg gcgctgccct ggccggcaag ctgaggagcg |
| | gcgaccgcag catggtggag |
| 241 | gtgctggccg accacccggg cgagctggtg cgcaccgaca |
| | gccccaactt cctctgctcc |
| 301 | gtgctgccta cgcactggcg ctgcaacaag accctgccca |
| | tcgctttcaa ggtggtggcc |
| 361 | ctaggggatg ttccagatgg cactctggtc actgtgatgg |
| | ctggcaatga tgaaaactac |
| 421 | tcggctgagc tgagaaatgc taccgcagcc atgaagaacc |
| | aggttgcaag atttaatgac |
| 481 | ctcaggtttg tcggtcgaag tggaagaggg aaaagcttca |
| | ctctgaccat cactgtcttc |
| 541 | acaaacccac cgcaagtcgc cacctaccac agagccatca |
| | aaatcacagt ggatgggccc |
| 601 | cgagaacctc gaagacatcg gcagaaacta gatgatcaga |
| | ccaagcccgg gagcttgtcc |
| 661 | ttttccgagc ggctcagtga actggagcag ctgcggcgca |
| | cagccatgag ggtcagccca |
| 721 | caccacccag cccccacgcc caaccctcgt gcctccctga |
| | accactccac tgcctttaac |
| 781 | cctcagcctc agagtcagat gcaggataca aggcagatcc |
| | aaccatcccc accgtggtcc |
| 841 | tacgatcagt cctaccaata cctgggatcc attgcctctc |
| | cttctgtgca cccagcaacg |
| 901 | cccatttcac ctggacgtgc cagcggcatg acaaccctct |
| | ctgcagaact ttccagtcga |
| 961 | ctctcaacgg cacccgacct gacagcgttc agcgacccgc |
| | gccagttccc cgcgctgccc |
| 1021 | tccatctccg acccccgcat gcactatcca ggcgccttca |
| | cctactcccc gacgccggtc |
| 1081 | acctcgggca tcggcatcgg catgtcggcc atgggctcgg |
| | ccacgcgcta ccacacctac |
| 1141 | ctgccgccgc cctaccccgg ctcgtcgcaa gcgcagggag |
| | gcccgttcca agccagctcg |
| 1201 | ccctcctacc acctgtacta cggcgcctcg gccggctcct |
| | accagttctc catggtgggc |
| 1261 | ggcgagcgct cgccgccgcg catcctgccg ccctgcacca |
| | acgcctccac cggctccgcg |
| 1321 | ctgctcaacc ccagcctccc gaaccagagc gacgtggtgg |
| | aggccgaggg cagccacagc |
| 1381 | aactccccca ccaacatggc gccctccgcg cgcctggagg |
| | aggccgtgtg gaggccctac |
| 1441 | tga |

RUNX1 Protein (Homo sapiens)

SEQ ID NO: 12

| | |
|---|---|
| 1 | masdsifesf psypqcfmre cilgmnpsrd vhdastsrrf |
| | tppstalspg kmsealplga |
| 61 | pdagaalagk lrsgdrsmve vladhpgelv rtdspnflcs |
| | vlpthwrcnk tlpiafkvva |
| 121 | lgdvpdgtlv tvmagndeny saelrnataa mknqvarfnd |
| | lrfvgrsgrg ksftltitvf |
| 181 | tnppqvatyh raikitvdgp reprrhrqkl ddqtkpgsls |
| | fserlseleq lrrtamrvsp |
| 241 | hhpaptpnpr aslnhstafn pqpqsqmqdt rqiqpsppws |
| | ydqsyqylgs iaspsvhpat |
| 301 | pispgrasgm ttlsaelssr lstapdltaf sdprqfpalp |
| | sisdprmhyp gaftysptpv |
| 361 | tsgigigmsa mgsatryhty lpppypgssq aqggpfqass |
| | psyhlyygas agsyqfsmvg |
| 421 | gerspprilp pctnastgsa llnpslpnqs dvveaegshs |
| | nsptnmapsa rleeavwrpy |

Ets2 cDNA (Homo sapiens)

SEQ ID NO: 13

| | |
|---|---|
| 1 | atgaatgatt tcggaatcaa gaatatggac caggtagccc |
| | ctgtggctaa cagttacaga |
| 61 | gggacactca agcgccagcc agcctttgac acctttgatg |
| | ggtccctgtt tgctgttttt |
| 121 | ccttctctaa atgaagagca aacactgcaa gaagtgccaa |
| | caggcttgga ttccatttct |
| 181 | catgactccg ccaactgtga attgcctttg ttaaccccgt |
| | gcagcaaggc tgtgatgagt |
| 241 | caagccttaa aagctacctt cagtggcttc aaaaaggaac |
| | agcggcgcct gggcattcca |
| 301 | aagaacccct ggctgtggag tgagcaacag gtatgccagt |
| | ggcttctctg ggccaccaat |
| 361 | gagttcagtc tggtgaacgt gaatctgcag aggttcggca |
| | tgaatggcca gatgctgtgt |
| 421 | aaccttggca aggaacgctt tctggagctg gcacctgact |
| | ttgtgggtga cattctctgg |
| 481 | gaacatctgg agcaaatgat caaagaaaac caagaaaaga |
| | cagaagatca atatgaagaa |
| 541 | aattcacacc tcacctccgt tcctcattgg attaacagca |
| | atacattagg ttttggcaca |
| 601 | gagcaggcgc cctatggaat gcagacacag aattacccca |
| | aaggcggcct cctggacagc |
| 661 | atgtgtccgg cctccacacc cagcgtactc agctctgagc |
| | aggagtttca gatgttcccc |
| 721 | aagtctcggc tcagctccgt cagcgtcacc tactgctctg |
| | tcagtcagga cttcccaggc |
| 781 | agcaacttga atttgctcac caacaattct gggactccca |
| | aagaccacga ctcccctgag |
| 841 | aacggtgcgg acagcttcga gagctcagac tccctcctcc |
| | agtcctggaa cagccagtcg |
| 901 | tccttgctgg atgtgcaacg ggttccttcc ttcgagagct |
| | tcgaagatga ctgcagccag |
| 961 | tctctctgcc tcaataagcc aaccatgtct ttcaaggatt |
| | acatccaaga gaggagtgac |
| 1021 | ccagtggagc aaggcaaacc agttatacct gcagctgtgc |
| | tggccggctt cacaggaagt |
| 1081 | ggacctattc agctgtggca gtttctcctg gagctgctat |
| | cagacaaatc ctgccagtca |
| 1141 | ttcatcagct ggactggaga cggatgggag tttaagctcg |
| | ccgaccccga tgaggtggcc |
| 1201 | cgccggtggg gaaagaggaa aaataagccc aagatgaact |
| | acgagaagct gagccggggc |
| 1261 | ttacgctact attacgacaa gaacatcatc cacaagacgt |
| | cggggaagcg ctacgtgtac |
| 1321 | cgcttcgtgt gcgacctcca gaacttgctg gggttcacgc |
| | ccgaggaact gcacgccatc |
| 1381 | ctgggcgtcc agcccgacac ggaggactga |

ETS2 Protein (Homo sapiens)

SEQ ID NO: 14

| | |
|---|---|
| 1 | mndfgiknmd qvapvansyr gtlkrqpafd tfdgslfavf |
| | pslneeqtlq evptgldsis |
| 61 | hdsancelpl ltpcskavms qalkatfsgf kkeqrrlgip |
| | knpwlwseqq vcqwllwatn |
| 121 | efslvnvnlq rfgmngqmlc nlgkerflel apdfvgdilw |
| | ehleqmiken qektedgyee |
| 181 | nshltsvphw insntlgfgt eqapygmqtq nypkggllds |
| | mcpastpsvl sseqefqmfp |
| 241 | ksrlssvsvt ycsvsqdfpg snlnlltnns gtpkdhdspe |
| | ngadsfessd sllqswnsqs |
| 301 | slldvqrvps fesfeddcsq slclnkptms fkdyiqersd |
| | pveqgkpvip aavlagftgs |
| 361 | gpiqlwqfll ellsdkscqs fiswtgdgwe fkladpdeva |
| | rrwgkrknkp kmnyeklsrg |
| 421 | lryyydknii hktsgkryvy rfvcdlqnll gftpeelhai |
| | lgvqpdted |

Tmprss2 cDNA (Homo sapiens)

SEQ ID NO: 15

| | |
|---|---|
| 1 | atggctttga actcagggtc accaccagct attggacctt |
| | actatgaaaa ccatggatac |
| 61 | caaccggaaa acccctatcc cgcacagccc actgtggtcc |
| | ccactgtcta cgaggtgcat |
| 121 | ccggctcagt actacccgtc ccccgtgccc cagtacgccc |
| | cgagggtcct gacgcaggct |
| 181 | tccaaccccg tcgtctgcac gcagcccaaa tccccatccg |
| | ggacagtgtg cacctcaaag |
| 241 | actaagaaag cactgtgcat caccttgacc ctggggacct |
| | tcctcgtggg agctgcgctg |
| 301 | gccgctggcc tactctggaa gttcatgggc agcaagtgct |
| | ccaactctgg gatagagtgc |
| 361 | gactcctcag gtacctgcat caacccctct aactggtgtg |
| | atggcgtgtc acactgcccc |
| 421 | ggcggggagg acgagaatcg gtgtgttcgc ctctacggac |
| | caaacttcat ccttcagatg |
| 481 | tactcatctc agaggaagtc ctggcaccct gtgtgccaag |
| | acgactggaa cgagaactac |
| 541 | gggcgggcgg cctgcaggga catgggctat aagaataatt |
| | tttactctag ccaaggaata |
| 601 | gtggatgaca gcggatccac cagctttatg aaactgaaca |
| | caagtgccgg caatgtcgat |
| 661 | atctataaaa aactgtacca cagtgatgcc tgttcttcaa |
| | aagcagtggt ttctttacgc |
| 721 | tgtatagcct gcggggtcaa cttgaactca agccgccaga |
| | gcaggatcgt gggcggtgag |
| 781 | agcgcgctcc cgggggcctg gccctggcag gtcagcctgc |
| | acgtccagaa cgtccacgtg |
| 841 | tgcggaggct ccatcatcac ccccgagtgg atcgtgacag |
| | ccgcccactg cgtggaaaaa |
| 901 | cctcttaaca atccatggca ttggacggca tttgcgggga |
| | ttttgagaca atctttcatg |
| 961 | ttctatggag ccggatacca agtagaaaaa gtgatttctc |
| | atccaaatta tgactccaag |
| 1021 | accaagaaca atgacattgc gctgatgaag ctgcagaagc |
| | ctctgacttt caacgaccta |
| 1081 | gtgaaaccag tgtgtctgcc caacccaggc atgatgctgc |
| | agccagaaca gctctgctgg |
| 1141 | atttccgggt ggggggccac cgaggagaaa gggaagacct |
| | cagaagtgct gaacgctgcc |
| 1201 | aaggtgcttc tcattgagac acagagatgc aacagcagat |
| | atgtctatga caacctgatc |
| 1261 | acaccagcca tgatctgtgc cggcttcctg caggggaacg |
| | tcgattcttg ccagggtgac |
| 1321 | agtggagggc ctctggtcac ttcgaagaac aatatctggt |
| | ggctgatagg ggatacaagc |
| 1381 | tggggttctg gctgtgccaa agcttacaga ccaggagtgt |
| | acgggaatgt gatggtattc |
| 1441 | acggactgga tttatcgaca aatgagggca gacggctaa |

TMPRSS2 Protein (Homo sapiens)

SEQ ID NO: 16

| | |
|---|---|
| 1 | malnsgsppa igpyyenhgy qpenpypaqp tvvptvyevh |
| | paqyypspvp qyaprvltqa |
| 61 | snpvvctqpk spsgtvctsk tkkalcitlt lgtflvgaal |
| | aagllwkfmg skcsnsgiec |
| 121 | dssgtcinps nwcdgvshcp ggedenrcvr lygpnfilqm |
| | yssqrkswhp vcqddwneny |
| 181 | graacrdmgy knnfyssqgi vddsgstsfm klntsagnvd |
| | iykklyhsda csskavvslr |
| 241 | ciacgvnlns srqsrivgge salpgawpwq vslhvqnvhv |
| | cggsiitpew ivtaahcvek |
| 301 | plnnpwhwta fagilrqsfm fygagyqvek vishpnydsk |
| | tknndialmk lqkpltfndl |
| 361 | vkpvclpnpg mmlqpeqlcw isgwgateek gktsevlnaa |
| | kvllietqrc nsryvydnli |
| 421 | tpamicagfl qgnvdscqgd sggplvtskn niwwligdts |
| | wgsgcakayr pgvygnvmvf |
| 481 | tdwiyrqmra dg |

Ripk4 cDNA (Homo sapiens)

| SEQ ID NO: 17 | |
|---|---|
| 1 | atggagggcg acggcgggac cccatgggcc ctggcgctgc tgcgcacctt cgacgcgggc |
| 61 | gagttcacgg gctgggagaa ggtgggctcg ggcggcttcg ggcaggtgta |
| | caaggtgcgc |
| 121 | catgtccact ggaagacctg gctggccatc aagtgctcgc ccagcctgca cgtcgacgac |
| 181 | agggagcgca tggagctttt ggaagaagcc aagaagatgg agatggccaa |
| | gtttcgctac |
| 241 | atcctgcctg tgtatggcat ctgccgcgaa cctgtcggcc tggtcatgga gtacatggag |
| 301 | acgggctccc tggaaaagct gctggcttcg gagccattgc catgggatct |
| | ccggttccga |
| 361 | atcatccacg agacggcggt gggcatgaac ttcctgcact gcatggcccc |
| | gccactcctg |
| 421 | cacctggacc tcaagcccgc gaacatcctg ctggatgccc actaccacgt |
| | caagatttct |
| 481 | gattttggtc tggccaagtg caacgggctg tcccactcgc atgacctcag catggatggc |
| 541 | ctgtttggca caatcgccta cctccctcca gagcgcatca gggagaagag |
| | ccggctcttc |
| 601 | gacaccaagc acgatgtata cagctttgcg atcgtcatct ggggcgtgct |
| | cacacagaag |
| 661 | aagccgtttg cagatgagaa gaacatcctg cacatcatgg tgaaggtggt |
| | gaagggccac |
| 721 | cgccccgagc tgccgcccgt gtgcagagcc cggccgcgcg cctgcagcca |
| | cctgatacgc |
| 781 | ctcatgcagc ggtgctggca gggggatccg cgagttaggc ccaccttcca |
| | agaaattact |
| 841 | tctgaaaccg aggacctgtg tgaaaagcct gatgacgaag tgaaagaaac |
| | tgctcatgat |
| 901 | ctggacgtga aaagcccccc ggagcccagg agcgaggtgg tgcctgcgag |
| | gctcaagcgg |
| 961 | gcctctgccc ccaccttcga taacgactac agcctctccg agctgctctc acagctggac |
| 1021 | tctggagttt cccaggctgt cgagggcccc gaggagctca gccgcagctc |
| | ctctgagtcc |
| 1081 | aagctgccat cgtccggcag tgggaagagg ctctcggggg tgtcctcggt |
| | ggactccgcc |
| 1141 | ttctcttcca gaggatcact gtcgctgtcc tttgagcggg aaccttcaac cagcgatctg |
| 1201 | ggcaccacag acgtccagaa gaagaagctt gtggatgcca tcgtgtccgg ggacaccagc |
| 1261 | aaactgatga agatcctgca gccgcaggac gtggacctgg cactggacag |
| | cggtgccagc |
| 1321 | ctgctgcacc tggcggtgga ggccgggcaa gaggagtgcg ccaagtggct gctgctcaac |
| 1381 | aatgccaacc ccaacctgag caaccgtagg ggctccaccc cgttgcacat |
| | ggccgtggag |
| 1441 | aggagggtgc ggggtgtcgt ggagctcctg ctggcgcgga agatcagtgt caacgccaag |
| 1501 | gatgaggacc agtggacagc cctccacttt gcagcccaga acggggacga |
| | gtctagcaca |
| 1561 | cggctgctgt tggagaagaa cgcctcggtc aacgaggtgg actttgaggg |
| | ccggacgccc |
| 1621 | atgcacgtgg cctgccagca cgggcaggag aatatcgtgc gcatcctgct gcgccgaggc |
| 1681 | gtggacgtga gcctgcaggg caaggatgcc tggctgccac tgcactacgc |
| | tgcctggcag |
| 1741 | ggccacctgc ccatcgtcaa gctgctggcc aagcagccgg gggtgagtgt gaacgcccag |
| 1801 | acgctggatg ggaggacgcc attgcacctg gccgcacagc gcgggcacta |
| | ccgcgtggcc |
| 1861 | cgcatcctca tcgacctgtg ctccgacgtc aacgtctgca gcctgctggc acagacaccc |
| 1921 | ctgcacgtgg ccgcggagac ggggcacacg agcactgcca ggctgctcct |
| | gcatcggggc |
| 1981 | gctggcaagg aggccatgac ctcagacggc tacaccgctc tgcacctggc tgcccgcaac |
| 2041 | ggacacctgg ccactgtcaa gctgcttgtc gaggagaagg ccgatgtgct |
| | ggcccgggga |
| 2101 | cccctgaacc agacggcgct gcacctggct gccgcccacg ggcactcgga |
| | ggtggtggag |
| 2161 | gagttggtca gcgccgatgt cattgacctg ttcgacgagc aggggctcag cgcgctgcac |
| 2221 | ctggccgccc agggccggca cgcacagacg gtggagactc tgctcaggca |
| | tggggcccac |
| 2281 | atcaacctgc agagcctcaa gttccagggc ggccatggcc ccgccgccac gctcctgcgg |
| 2341 | cgaagcaaga cctag |

RIPK4 Protein (Homo sapiens)

SEQ ID NO: 18

| | |
|---|---|
| 1 | megdggtpwa lallrtfdag eftgwekvgs ggfgqvykvr |
| | hvhwktwlai kcspslhvdd |
| 61 | rermelleea kkmemakfry ilpvygicre pvglvmeyme |
| | tgslekllas eplpwdlrfr |
| 121 | iihetavgmn flhcmappll hldlkpanil ldahyhvkis |
| | dfglakcngl shshdlsmdg |
| 181 | lfgtiaylpp erireksrlf dtkhdvysfa iviwgvltqk |
| | kpfadeknil himvkvvkgh |
| 241 | rpelppvcra rpracshlir lmqrcwqgdp rvrptfqeit |
| | setedlcekp ddevketahd |
| 301 | ldvksppepr sevvparlkr asaptfdndy slsellsqld |
| | sgvsqavegp eelsrssses |
| 361 | klpssgsgkr lsgvssvdsa fssrgslsls ferepstsdl |
| | gttdvqkkkl vdaivsgdts |
| 421 | klmkilqpqd vdlaldsgas llhlaveagq eecakwllln |
| | nanpnlsnrr gstplhmave |
| 481 | rrvrgvvell larkisvnak dedqwtalhf aaqngdesst |
| | rllleknasv nevdfegrtp |
| 541 | mhvacqhgqe nivrillrrg vdvslqgkda wlplhyaawq |
| | ghlpivklla kqpgvsvnaq |
| 601 | tldgrtplhl aaqrghyrva rilidlcsdv nvcsllaqtp |
| | lhvaaetght starlllhrg |
| 661 | agkeamtsdg ytalhlaarn ghlatvkllv eekadvlarg |
| | plnqtalhla aahghsevve |
| 721 | elvsadvidl fdeqglsalh laaqgrhaqt vetllrhgah |
| | inlqslkfqg ghgpaatllr |
| 781 | rskt |

Erg cDNA (Homo sapiens)

SEQ ID NO: 19

| | |
|---|---|
| 1 | atggccagca ctattaagga agccttatca gttgtgagtg |
| | aggaccagtc gttgtttgag |
| 61 | tgtgcctacg gaacgccaca cctggctaag acagagatga |
| | ccgcgtcctc ctccagcgac |
| 121 | tatggacaga cttccaagat gagcccacgc gtccctcagc |
| | aggattggct gtctcaaccc |
| 181 | ccagccaggg tcaccatcaa aatggaatgt aaccctagcc |
| | aggtgaatgg ctcaaggaac |
| 241 | tctcctgatg aatgcagtgt ggccaaaggc gggaagatgg |
| | tgggcagccc agacaccgtt |
| 301 | gggatgaact acggcagcta catggaggag aagcacatgc |
| | cacccccaaa catgaccacg |
| 361 | aacgagcgca gagttatcgt gccagcagat cctacgctat |
| | ggagtacaga ccatgtgcgg |
| 421 | cagtggctgg agtgggcggt gaaagaatat ggccttccag |
| | acgtcaacat cttgttattc |
| 481 | cagaacatcg atgggaagga actgtgcaag atgaccaagg |
| | acgacttcca gaggctcacc |
| 541 | cccagctaca acgccgacat ccttctctca catctccact |
| | acctcagaga gactcctctt |
| 601 | ccacatttga cttcagatga tgttgataaa gccttacaaa |
| | actctccacg gttaatgcat |
| 661 | gctagaaaca cagggggtgc agcttttatt ttcccaaata |
| | cttcagtata tcctgaagct |
| 721 | acgcaaagaa ttacaactag gccagattta ccatatgagc |
| | cccccaggag atcagcctgg |
| 781 | accggtcacg gccaccccac gccccagtcg aaagctgctc |
| | aaccatctcc ttccacagtg |
| 841 | cccaaaactg aagaccagcg tcctcagtta gatccttatc |
| | agattcttgg accaacaagt |
| 901 | agccgccttg caaatccagg cagtggccag atccagcttt |
| | ggcagttcct cctggagctc |
| 961 | ctgtcggaca gctccaactc cagctgcatc acctgggaag |
| | gcaccaacgg ggagttcaag |
| 1021 | atgacggatc ccgacgaggt ggcccggcgc tggggagagc |
| | ggaagagcaa acccaacatg |
| 1081 | aactacgata agctcagccg cgccctccgt tactactatg |
| | acaagaacat catgaccaag |
| 1141 | gtccatggga agcgctacgc ctacaagttc gacttccacg |
| | ggatcgccca ggccctccag |
| 1201 | ccccaccccc cggagtcatc tctgtacaag tacccctcag |
| | acctcccgta catgggctcc |
| 1261 | tatcacgccc acccacagaa gatgaacttt gtggcgcccc |
| | accctccagc cctccccgtg |
| 1321 | acatcttcca gtttttttgc tgccccaaac ccatactgga |
| | attcaccaac tgggggtata |
| 1381 | taccccaaca ctaggctccc caccagccat atgccttctc |
| | atctgggcac ttactactaa |

ERG Protein (Homo sapiens)

SEQ ID NO: 20

| | |
|---|---|
| 1 | mastikeals vvsedqslfe caygtphlak temtassssd |
| | ygqtskmspr vpqqdwlsqp |
| 61 | parvtikmec npsqvngsrn spdecsvakg gkmvgspdtv |
| | gmnygsymee khmpppnmtt |
| 121 | nerrvivpad ptlwstdhvr qwlewavkey glpdvnillf |
| | qnidgkelck mtkddfqrlt |
| 181 | psynadills hlhylretpl phltsddvdk alqnsprlmh |
| | arntggaafi fpntsvypea |
| 241 | tqrittrpdl pyepprrsaw tghghptpqs kaaqpspstv |
| | pktedqrpql dpyqilgpts |
| 301 | srlanpgsgq iqlwqfllel lsdssnssci twegtngefk |
| | mtdpdevarr wgerkskpnm |
| 361 | nydklsralr yyydknimtk vhgkryaykf dfhgiaqalq |
| | phppesslyk ypsdlpymgs |
| 421 | yhahpqkmnf vaphppalpv tsssffaapn pywnsptggi |
| | ypntrlptsh mpshlgtyy |

Gnb2 cDNA (Homo sapiens)

SEQ ID NO: 21

| | |
|---|---|
| 1 | atgagtgagc tggagcaact gagacaggag gccgagcagc |
| | tccggaacca gatccgggat |
| 61 | gcccgaaaag catgtgggga ctcaacactg acccagatca |
| | cagctgggct ggacccagtg |
| 121 | gggagaatcc agatgaggac ccggaggacc ctccgtgggc |
| | acctggcaaa gatctatgcc |
| 181 | atgcactggg ggaccgactc aaggctgctg gtcagcgcct |
| | cccaggatgg gaagctcatc |
| 241 | atctgggaca gctacaccac caacaaggtc cacgccatcc |
| | cgctgcgctc ctcctgggta |
| 301 | atgacctgtg cctacgcgcc ctcagggaac tttgtggcct |
| | gtggggggtt ggacaacatc |
| 361 | tgctccatct acagcctcaa gacccgcgag ggcaacgtca |
| | gggtcagccg ggagctgcct |
| 421 | ggccacactg ggtacctgtc gtgttgccgc ttcctggatg |
| | acaaccaaat catcaccagc |
| 481 | tctggggata ccacctgtgc cctgtgggac attgagacag |
| | gccagcagac agtgggtttt |
| 541 | gctggacaca gtggggatgt gatgtccctg tccctggccc |
| | ccgatggccg cacgtttgtg |
| 601 | tcaggcgcct gtgatgcctc tatcaagctg tgggacgtgc |
| | gggattccat gtgccgacag |
| 661 | accttcatcg gccatgaatc cgacatcaat gcagtggctt |
| | tcttccccaa cggctacgcc |
| 721 | ttcaccaccg gctctgacga cgccacgtgc cgcctcttcg |
| | acctgcgggc cgatcaggag |
| 781 | ctcctcatgt actcccatga caacatcatc tgtggcatca |
| | cctctgttgc cttctcgcgc |
| 841 | agcggacggc tgctgctcgc tggctacgac gacttcaact |
| | gcaacatctg ggatgccatg |
| 901 | aagggcgacc gtgcaggagt cctcgctggc cacgacaacc |
| | gcgtgagctg cctcggggtc |
| 961 | accgacgatg gcatggctgt ggccacgggc tcctgggact |
| | ccttcctcaa gatctggaac |
| 1021 | taa |

GNB2 Protein (Homo sapiens)

SEQ ID NO: 22

| | |
|---|---|
| 1 | mseleqlrqe aeqlrnqird arkacgdstl tqitagldpv |
| | griqmrtrrt lrghlakiya |
| 61 | mhwgtdsrll vsasqdgkli iwdsyttnkv haiplrsswv |
| | mtcayapsgn fvacggldni |
| 121 | csiyslktre gnvrvsrelp ghtgylsccr flddnqiits |
| | sgdttcalwd ietgqqtvgf |
| 181 | aghsgdvmsl slapdgrtfv sgacdasikl wdvrdsmcrq |
| | tfighesdin avaffpngya |
| 241 | fttgsddatc rlfdlradqe llmyshdnii cgitsvafsr |
| | sgrlllagyd dfncniwdam |
| 301 | kgdragvlag hdnrvsclgv tddgmavatg swdsflkiwn |

Perq1 cDNA (Homo sapiens)

| SEQ ID NO: 23 | |
|---|---|
| 1 | atggcagcag agacactcaa ctttgggcct gagtggctca gggccctgtc cgggggcggc |
| 61 | agcgtggcct ccccaccccc gtcccctgcc atgcccaaat acaagctggc |
| | tgactaccgt |
| 121 | tatgggcgag aggaaatgct ggctctctac gtcaaggaga acaaggtccc ggaagagctg |
| 181 | caggacaagg agttcgccgc ggtgctgcag gacgagccac tgcagcccct |
| | ggctctggag |
| 241 | ccgctgactg aggaggaaca gagaaacttc tccctgtcag tgaacagcgt ggctgtgctg |
| 301 | aggctgatgg ggaaaggggc tggccccccc ctggctggca cctcccgagg |
| | caggggcagc |
| 361 | acgcggagcc gaggccgcgg ccgtggtgac agctgctttt accaaagaag catcgaagaa |
| 421 | ggcgatgggg cctttggacg aagcccccgg gaaatccagc gcagccagag |
| | ctgggatgac |
| 481 | agaggcgaga ggcggtttga gaagtcagca aggcgggatg gagcacgatg |
| | tggctttgag |
| 541 | gagggagggg ctggcccaag gaaggagcac gcccgctcag acagcgagaa |
| | ctggcgctcc |
| 601 | ctacgggagg aacaggagga ggaggaggag ggcagctgga ggctcggagc |
| | agggccccgg |
| 661 | cgagacggcg accgctggcg ctccgccagc cctgatggtg gtccccgctc tgctggctgg |
| 721 | cgggaacatg gggaacggcg gcgcaagttt gaatttgatt tgcgagggga tcgaggaggg |
| 781 | tgtggtgaag aggaggggcg gggaggggga ggcagctctc acctgcggcg |
| | gtgccgagcg |
| 841 | cctgaaggct ttgaggagga caaggatggg ctcccagagt ggtgcctgga cgatgaggat |
| 901 | gaagaaatgg gcacctttga tgcctctggg gccttcttgc ctctcaagaa gggccccaag |
| 961 | gagcccattc ctgaggagca ggagctggac ttccaagggt tggaggagga ggaggaacct |
| 1021 | tccgaagggc tagaggagga agggcctgag gcaggtggga aagagctgac |
| | cccactgcct |
| 1081 | cctcaggagg agaagtccag ctccccatcc ccactgccca ccctgggccc |
| | actctggggg |
| 1141 | acaaacgggg atggggacga aactgcagag aaagagcccc cagcggccga |
| | agatgatatt |
| 1201 | cgggggatcc agctgagtcc cggggtgggc tcctctgctg gcccacccgg |
| | agatctggag |
| 1261 | gatgatgaag gcttgaagca cctgcagcag gaggcggaga agctggtggc |
| | ctccctgcag |
| 1321 | gacagctcct tggaggagga gcagttcacg gctgccatgc agacccaggg |
| | cctgcgccac |
| 1381 | tctgcagccg ccactgccct cccgctcagc catggggctg cccggaagtg gttctacaag |
| 1441 | gacccacagg gcgagatcca aggccccttc acgacacagg agatggcaga |
| | gtggttccag |
| 1501 | gccggctact tttccatgtc actgctggtg aagcggggct gcgatgaggg cttccagccg |
| 1561 | ctgggcgagg tgatcaagat gtggggccgc gtgccctttg ccccagggcc ctcacctccc |
| 1621 | ccactgctgg gaaacatgga ccaggagcgg ctgaagaagc aacaggagct |
| | ggccgcggcg |
| 1681 | gccttgtacc agcagctgca gcaccagcag tttctccagc tggtcagcag ccgccagctc |
| 1741 | ccgcagtgcg cgctccgaga aaaggcagct ctgggggacc tgacaccgcc |
| | accaccgccg |
| 1801 | ccgccacagc agcagcagca gcagctcacg gcattcctgc agcagctcca |
| | ggcgctcaaa |
| 1861 | ccccccagag gcggggacca gaacctgctc ccgacgatga gccggtcctt gtcggtgcca |
| 1921 | gattcgggcc gcctctggga cgtacatacc tcagcctcat cacagtcagg tggtgaggcc |
| 1981 | agtctttggg acataccaat taactcttcg actcagggtc caattctaga acaactccag |
| 2041 | ctgcaacata aattccagga gcgcagagaa gtggagctca gggcgaagcg |
| | ggaggaagag |
| 2101 | gaacgcaagc gtcgagagga gaagcgccgc cagcagcagc aggaggagca |
| | gaagcggcgg |
| 2161 | caggaggagg aagagctgtt tcggcgcaag cacgtgcggc agcaggagct |
| | attgctgaag |
| 2221 | ttgctacagc agcagcaggc ggtccctgtg ccccccgcac ccagctcccc gcccccactc |
| 2281 | tgggctggcc tggccaagca ggggctgtcc atgaagacgc tcctggagtt gcagctggag |
| 2341 | ggcgagcggc agctgcacaa acagccccca cctcgggagc cagctcgggc |
| | ccaggccccc |
| 2401 | aaccaccgag tgcagcttgg gggcctgggc actgcccccc tgaaccagtg |
| | ggtgtctgag |
| 2461 | gctgggccac tgtggggcgg gccagacaag agtgggggcg gcagcagcgg |
| | cctggggctc |
| 2521 | tgggaggaca cccccaagag cggcgggagc ctggtccgtg gcctcggcct |
| | gaagaacagc |
| 2581 | cggagcagcc catctctcag tgactcatac agccacctat cgggtcggcc cattcgcaaa |
| 2641 | aagacggagg aagaagagaa gctgctgaag ctgctgcagg gcattcccag |
| | gccccaggac |
| 2701 | ggcttcaccc agtggtgcga gcagatgctg cacacgctga gcgccacggg cagcctggac |
| 2761 | gtgcccatgg ctgtagcgat cctcaaggag gtggaatccc cctatgatgt ccacgattat |
| 2821 | atccgttcct gcctggggga cacgctggaa gccaaagaat ttgccaaaca attcctggag |
| 2881 | cggagggcca agcagaaagc cagccagcag cggcagcagc agcaggaggc |
| | atggctgagc |
| 2941 | agcgcctcgc tgcagacggc cttccaggcc aaccacagca ccaaactcgg |
| | ccccggggag |
| 3001 | ggcagcaagg ccaagaggcg ggcactgatg ctgcactcag accccagcat |
| | cctggggtac |
| 3061 | tccctgcacg gatcttctgg tgagatcgag agcgtggatg actactga |

PERQ1 Protein (Homo sapiens)

SEQ ID NO: 24

| | |
|---|---|
| 1 | maaetlnfgp ewlralsggg svaspppspa mpkykladyr |
| | ygreemlaly vkenkvpeel |
| 61 | qdkefaavlq deplqplale plteeeqrnf slsvnsvavl |
| | rlmgkgagpp lagtsrgrgs |
| 121 | trsrgrgrgd scfyqrsiee gdgafgrspr eiqrsqswdd |
| | rgerrfeksa rrdgarcgfe |
| 181 | eggagprkeh arsdsenwrs lreeqeeeee gswrlgagpr |
| | rdgdrwrsas pdggprsagw |
| 241 | rehgerrrkf efdlrgdrgg cgeeegrggg gsshlrrcra |
| | pegfeedkdg lpewcldded |
| 301 | eemgtfdasg aflplkkgpk epipeeqeld fqgleeeeep |
| | segleeegpe aggkeltplp |
| 361 | pqeeksssps plptlgplwg tngdgdetae keppaaeddi |
| | rgiqlspgvg ssagppgdle |
| 421 | ddeglkhlqq eaeklvaslq dssleeeqft aamqtqglrh |
| | saaatalpls hgaarkwfyk |
| 481 | dpqgeiqgpf ttqemaewfq agyfsmsllv krgcdegfqp |
| | lgevikmwgr vpfapgpspp |
| 541 | pllgnmdqer lkkqqelaaa alyqqlqhqq flqlvssrql |
| | pqcalrekaa lgdltppppp |
| 601 | ppqqqqqqlt aflqqlqalk pprggdqnll ptmsrslsvp |
| | dsgrlwdvht sassqsggea |
| 661 | slwdipinss tqgpileqlq lqhkfqerre velrakreee |
| | erkrreekrr qqqqeeqkrr |
| 721 | qeeeelfrrk hvrqqelllk llqqqqavpv ppapsspppl |
| | waglakqgls mktllelqle |
| 781 | gerqlhkqpp preparaqap nhrvqlgglg taplnqwvse |
| | agplwggpdk sgggssglgl |
| 841 | wedtpksggs lvrglglkns rsspslsdsy shlsgrpirk |
| | kteeeekllk llqgiprpqd |
| 901 | gftqwceqml htlsatgsld vpmavailke vespydvhdy |
| | irsclgdtle akefakqfle |
| 961 | rrakqkasqq rqqqqeawls saslqtafqa nhstklgpge |
| | gskakrralm lhsdpsilgy |
| 1021 | slhgssgeie svddy |

Tox cDNA (Homo sapiens)

| SEQ ID NO: 25 | |
|---|---|
| 1 | atggacgtaa gattttatcc acctccagcc cagcccgccg ctgcgcccga |
| | cgctccctgt |
| 61 | ctgggacctt ctccctgcct ggacccctac tattgcaaca agtttgacgg |
| | tgagaacatg |
| 121 | tatatgagca tgacagagcc gagccaggac tatgtgccag ccagccagtc |
| | ctaccctggt |
| 181 | ccaagcctgg aaagtgaaga cttcaacatt ccaccaatta ctcctccttc |
| | cctcccagac |
| 241 | cactcgctgg tgcacctgaa tgaagttgag tctggttacc attctctgtg |
| | tcaccccatg |
| 301 | aaccataatg gcctgctacc atttcatcca caaaacatgg acctccctga |
| | aatcacagtc |
| 361 | tccaatatgc tgggccagga tggaacactg ctttctaatt ccatttctgt |
| | gatgccagat |
| 421 | atacgaaacc cagaaggaac tcagtacagt tcccatcctc agatggcagc |
| | catgagacca |
| 481 | aggggccagc ctgcagacat caggcagcag ccaggaatga tgccacatgg |
| | ccagctgact |
| 541 | accattaacc agtcacagct aagtgctcaa cttggtttga atatgggagg |
| | aagcaatgtt |
| 601 | ccccacaact caccatctcc acctggaagc aagtctgcaa ctccttcacc |
| | atccagttca |
| 661 | gtgcatgaag atgaaggcga tgatacctct aagatcaatg gtggagagaa |
| | gcggcctgcc |
| 721 | tctgatatgg ggaaaaaacc aaaaactccc aaaaagaaga agaagaagga |
| | tcccaatgag |
| 781 | ccccagaagc ctgtgtctgc ctatgcgtta ttctttcgtg atactcaggc |
| | cgccatcaag |
| 841 | ggccaaaatc caaacgctac ctttggcgaa gtctctaaaa ttgtggcttc |
| | aatgtgggac |
| 901 | ggtttaggag aagagcaaaa acaggtctat aaaaagaaaa ccgaggctgc |
| | gaagaaggag |
| 961 | tacctgaagc aactcgcagc atacagagcc agccttgtat ccaagagcta |
| | cagtgaacct |
| 1021 | gttgacgtga agacatctca acctcctcag ctgatcaatt cgaagccgtc |
| | ggtgttccat |
| 1081 | gggcccagcc aggcccactc ggccctgtac ctaagttccc actatcacca |
| | acaaccggga |
| 1141 | atgaatcctc acctaactgc catgcatcct agtctcccca ggaacatagc |
| | ccccaagccg |
| 1201 | aataaccaaa tgccagtgac tgtctctata gcaaacatgg ctgtgtcccc |
| | tcctcctccc |
| 1261 | ctccagatca gcccgcctct tcaccagcat ctcaacatgc agcagcacca |
| | gccgctcacc |
| 1321 | atgcagcagc cccttgggaa ccagctcccc atgcaggtcc agtctgcctt |
| | acactcaccc |
| 1381 | accatgcagc aaggatttac tcttcaaccc gactatcaga ctattatcaa |
| | tcctacatct |
| 1441 | acagctgcac aagttgtcac ccaggcaatg gagtatgtgc gttcggggtg |
| | cagaaatcct |
| 1501 | cccccacaac cggtggactg gaataacgac tactgcagta gtgggggcat |
| | gcagagggac |
| 1561 | aaagcactgt accttacttg a |

TOX Protein (Homo sapiens)

| SEQ ID NO: 26 | |
|---|---|
| 1 | mdvrfypppa qpaaapdapc lgpspcldpy ycnkfdgenm ymsmtepsqd |
| | yvpasqsypg |
| 61 | pslesedfni ppitppslpd hslvhlneve sgyhslchpm nhngllpfhp |
| | qnmdlpeitv |
| 121 | snmlgqdgtl lsnsisvmpd irnpegtqys shpqmaamrp rggpadirqq |
| | pgmmphgqlt |
| 181 | tinqsqlsaq lglnmggsnv phnspsppgs ksatpspsss vhedegddts |
| | kinggekrpa |
| 241 | sdmgkkpktp kkkkkkdpne pqkpvsayal ffrdtqaaik gqnpnatfge |
| | vskivasmwd |
| 301 | glgeeqkqvy kkkteaakke ylkqlaayra slvsksysep vdvktsqppq |
| | linskpsvfh |
| 361 | gpsqahsaly lsshyhqqpg mnphltamhp slprniapkp nnqmpvtvsi |
| | anmavspppp |
| 421 | lqispplhqh lnmqqhqplt mqqplgnqlp mqvqsalhsp tmqqgftlqp |
| | dyqtiinpts |
| 481 | taaqvvtqam eyvrsgcrnp ppqpvdwnnd ycssggmqrd kalylt |

Set cDNA (Homo sapiens)

| SEQ ID NO: 27 | |
|---|---|
| 1 | atggccccta aacgccagtc tccactcccg cctcaaaaga agaaaccaag |
| | accacctcct |
| 61 | gctctgggac cggaggagac atcggcctct gcaggcttgc cgaagaaggg |
| | agaaaaagaa |
| 121 | cagcaagaag cgattgaaca cattgatgaa gtacaaaatg aaatagacag |
| | acttaatgaa |
| 181 | caagccagtg aggagatttt gaaagtagaa cagaaatata acaaactccg |
| | ccaaccattt |
| 241 | tttcagaaga ggtcagaatt gatcgccaaa atcccaaatt tttgggtaac |
| | aacatttgtc |
| 301 | aaccatccac aagtgtctgc actgcttggg gaggaagatg aagaggcact |
| | gcattatttg |
| 361 | accagagttg aagtgacaga atttgaagat attaaatcag gttacagaat |
| | agatttttat |
| 421 | tttgatgaaa atccttactt tgaaaataaa gttctctcca aagaatttca |
| | tctgaatgag |
| 481 | agtggtgatc catcttcgaa gtccaccgaa atcaaatgga aatctggaaa |
| | ggatttgacg |
| 541 | aaacgttcga gtcaaacgca gaataaagcc agcaggaaga ggcagcatga |
| | ggaaccagag |
| 601 | agcttcttta cctggtttac tgaccattct gatgcaggtg ctgatgagtt |
| | aggagaggtc |
| 661 | atcaaagatg atatttggcc aaacccatta cagtactact tggttcccga |
| | tatggatgat |
| 721 | gaagaaggag aaggagaaga agatgatgat gatgatgaag aggaggaagg |
| | attagaagat |
| 781 | attgacgaag aaggggatga ggatgaaggt gaagaagatg aagatgatga |
| | tgaaggggag |
| 841 | gaaggagagg aggatgaagg agaagatgac taa |

SET Protein (Homo sapiens)

| SEQ ID NO: 28 | |
|---|---|
| 1 | mapkrqsplp pqkkkprppp algpeetsas aglpkkgeke qqeaiehide |
| | vqneidrlne |
| 61 | qaseeilkve qkynklrqpf fqkrseliak ipnfwvttfv nhpqvsallg |
| | eedeealhyl |
| 121 | trvevtefed iksgyridfy fdenpyfenk vlskefhlne sgdpsskste |
| | ikwksgkdlt |
| 181 | krssqtqnka srkrqheepe sfftwftdhs dagadelgev ikddiwpnpl |
| | qyylvpdmdd |
| 241 | eegegeeddd ddeeeegled ideegdedeg eededddege egeedegedd |

Fnbp1 cDNA (Homo sapiens)

| SEQ ID NO: 29 | |
|---|---|
| 1 | atgagctggg gcaccgagct ctgggatcag tttgacaact tagaaaaaca |
| | cacacagtgg |
| 61 | ggaattgata ttcttgagaa atatatcaag tttgtgaaag aaaggacaga |
| | gattgaactc |
| 121 | agctatgcaa agcaactcag gaatctttca aagaagtacc aacctaaaaa |
| | gaactcgaag |
| 181 | gaggaagaag aatacaagta tacgtcatgt aaagctttca tttccaacct |
| | gaacgaaatg |
| 241 | aatgattacg cagggcagca tgaagttatc tccgagaaca tggcatcaca |
| | gatcattgtg |
| 301 | gacttggcac gctatgttca ggaactgaaa caggagagga aatcaaactt |
| | tcacgatggc |
| 361 | cgtaaagcac agcagcacat cgagacttgc tggaagcagc ttgaatctag |
| | taaaaggcga |
| 421 | tttgaacgcg attgcaaaga ggcggacagg gcgcagcagt actttgagaa |
| | aatggacgct |
| 481 | gacatcaatg tcacaaaagc ggatgttgaa aaggcccgac aacaagctca |
| | aatacgtcac |
| 541 | caaatggcag aggacagcaa agcagattac tcatccattc tccagaaatt |
| | caaccatgag |
| 601 | cagcatgaat attaccatac tcacatcccc aacatcttcc agaaaataca |
| | agagatggag |
| 661 | gaaaggagga ttgtgagaat gggagagtcc atgaagacat atgcagaggt |
| | tgatcggcag |
| 721 | gtgatcccaa tcattgggaa gtgcctggat ggaatagtaa aagcagccga |
| | atcaattgat |
| 781 | cagaaaaatg attcacagct ggtaatagaa gcttataaat cagggtttga |
| | gcctcctgga |
| 841 | gacattgaat ttgaggatta cactcagcca atgaagcgca ctgtgtcaga |
| | taacagcctt |
| 901 | tcaaattcca gaggagaagg caaaccagac ctcaaatttg gtggcaaatc |
| | caaaggaaag |
| 961 | ttatggccgt tcatcaaaaa aaataagctt atgtcccttt taacatcccc |
| | ccatcagcct |
| 1021 | ccccctcccc ctcctgcctc tgcctcaccc tctgctgttc ccaacggccc |
| | ccagtctccc |
| 1081 | aagcagcaaa aggaacccct ctcccatcgc ttcaacgagt tcatgacctc |
| | caaacccaaa |
| 1141 | atccactgct tcaggagcct aaagcgtggg ctttctctca agctgggtgc |
| | aacaccggag |
| 1201 | gatttcagca acctcccacc tgaacaaaga aggaaaaagc tgcagcagaa |
| | agtcgatgag |
| 1261 | ttaaataaag aaattcagaa ggagatggat caaagagatg ccataacaaa |
| | aatgaaagat |
| 1321 | gtctacctaa agaatcctca gatgggagac ccagccagtt tggatcacaa |
| | attagcagaa |
| 1381 | gtcagccaaa atatagagaa actgcgagta gagacccaga aatttgaggc |
| | ctggctggct |
| 1441 | gaggttgaag gccggctccc agcacgcagc gagcaggcgc gccggcagag |
| | cggactgtac |
| 1501 | gacagccaga acccacccac agtcaacaac tgcgcccagg accgtgagag |
| | cccagatggc |
| 1561 | agttacacag aggagcagag tcaggagagt gagatgaagg tgctggccac |
| | ggattttgac |
| 1621 | gacgagtttg atgatgagga gcccctccct gccataggga cgtgcaaagc |
| | tctctacaca |
| 1681 | tttgaaggtc agaatgaagg aacgatttcc gtagttgaag gagaaacatt |
| | gtatgtcata |
| 1741 | gaggaagaca aaggcgatgg ctggacccgc attcggagaa atgaagatga |
| | agagggttat |
| 1801 | gtccccactt catatgtcga agtctgtttg gacaaaaatg ccaaagattc |
| | ctag |

FNBP1 Protein (Homo sapiens)

| SEQ ID NO: 30 | |
|---|---|
| 1 | mswgtelwdq fdnlekhtqw gidilekyik fvkerteiel syakqlrnls |
| | kkyqpkknsk |
| 61 | eeeeykytsc kafisnlnem ndyagqhevi senmasqiiv dlaryvqelk |
| | qerksnfhdg |
| 121 | rkaqqhietc wkqlesskrr ferdckeadr aqqyfekmda dinvtkadve |
| | karqqaqirh |
| 181 | qmaedskady ssilqkfnhe qheyyhthip nifqkiqeme errivrmges |
| | mktyaevdrq |
| 241 | vipiigkcld givkaaesid qkndsqlvie ayksgfeppg diefedytqp |
| | mkrtvsdnsl |
| 301 | snsrgegkpd lkfggkskgk lwpfikknkl mslltsphqp pppppasasp |
| | savpngpqsp |
| 361 | kqqkeplshr fnefmtskpk ihcfrslkrg lslklgatpe dfsnlppeqr |
| | rkklqqkvde |
| 421 | lnkeiqkemd qrdaitkmkd vylknpqmgd pasldhklae vsqnieklrv |
| | etqkfeawla |
| 481 | evegrlpars eqarrqsgly dsqnpptvnn caqdrespdg syteeqsqes |
| | emkvlatdfd |
| 541 | defddeeplp aigtckalyt fegqnegtis vvegetlyvi eedkgdgwtr |
| | irrnedeegy |
| 601 | vptsyvevcl dknakds |

Abl1 cDNA (Homo sapiens)

| SEQ ID NO: 31 | |
|---|---|
| 1 | atgttggaga tctgcctgaa gctggtgggc tgcaaatcca agaaggggct |
| | gtcctcgtcc |
| 61 | tccagctgtt atctggaaga agcccttcag cggccagtag catctgactt |
| | tgagcctcag |
| 121 | ggtctgagtg aagccgctcg ttggaactcc aaggaaaacc ttctcgctgg |
| | acccagtgaa |
| 181 | aatgacccca accttttcgt tgcactgtat gattttgtgg ccagtggaga |
| | taacactcta |
| 241 | agcataacta aaggtgaaaa gctccgggtc ttaggctata atcacaatgg |
| | ggaatggtgt |
| 301 | gaagcccaaa ccaaaaatgg ccaaggctgg gtcccaagca actacatcac |
| | gccagtcaac |
| 361 | agtctggaga aacactcctg gtaccatggg cctgtgtccc gcaatgccgc |
| | tgagtatctg |
| 421 | ctgagcagcg ggatcaatgg cagcttcttg gtgcgtgaga gtgagagcag |
| | tcctggccag |
| 481 | aggtccatct cgctgagata cgaagggagg gtgtaccatt acaggatcaa |
| | cactgcttct |
| 541 | gatggcaagc tctacgtctc ctccgagagc cgcttcaaca ccctggccga |
| | gttggttcat |
| 601 | catcattcaa cggtggccga cgggctcatc accacgctcc attatccagc |
| | cccaaagcgc |
| 661 | aacaagccca ctgtctatgg tgtgtccccc aactacgaca agtgggagat |
| | ggaacgcacg |
| 721 | gacatcacca tgaagcacaa gctgggcggg ggccagtacg gggaggtgta |
| | cgagggcgtg |
| 781 | tggaagaaat acagcctgac ggtggccgtg aagaccttga aggaggacac |
| | catggaggtg |
| 841 | gaagagttct tgaaagaagc tgcagtcatg aaagagatca aacaccctaa |
| | cctggtgcag |
| 901 | ctccttgggg tctgcacccg ggagcccccg ttctatatca tcactgagtt |
| | catgacctac |
| 961 | gggaacctcc tggactacct gagggagtgc aaccggcagg aggtgaacgc |
| | cgtggtgctg |
| 1021 | ctgtacatgg ccactcagat ctcgtcagcc atggagtacc tggagaagaa |
| | aaacttcatc |
| 1081 | cacagagatc ttgctgcccg aaactgcctg gtaggggaga accacttggt |
| | gaaggtagct |
| 1141 | gattttggcc tgagcaggtt gatgacaggg gacacctaca cagcccatgc |
| | tggagccaag |
| 1201 | ttccccatca aatggactgc acccgagagc ctggcctaca acaagttctc |
| | catcaagtcc |
| 1261 | gacgtctggg catttggagt attgctttgg gaaattgcta cctatggcat |
| | gtccccttac |
| 1321 | ccgggaattg acctgtccca ggtgtatgag ctgctagaga aggactaccg |
| | catggagcgc |
| 1381 | ccagaaggct gcccagagaa ggtctatgaa ctcatgcgag catgttggca |
| | gtggaatccc |
| 1441 | tctgaccggc cctcctttgc tgaaatccac caagcctttg aaacaatgtt |
| | ccaggaatcc |
| 1501 | agtatctcag acgaagtgga aaaggagctg gggaaacaag gcgtccgtgg |
| | ggctgtgagt |
| 1561 | accttgctgc aggccccaga gctgcccacc aagacgagga cctccaggag |
| | agctgcagag |
| 1621 | cacagagaca ccactgacgt gcctgagatg cctcactcca agggccaggg |
| | agagagcgat |
| 1681 | cctctggacc atgagcctgc cgtgtctcca ttgctccctc gaaaagagcg |
| | aggtcccccg |
| 1741 | gagggcggcc tgaatgaaga tgagcgcctt ctccccaaag acaaaaagac |
| | caacttgttc |
| 1801 | agcgccttga tcaagaagaa gaagaagaca gccccaaccc ctcccaaacg |
| | cagcagctcc |
| 1861 | ttccgggaga tggacggcca gccggagcgc agaggggccg gcgaggaaga |
| | gggccgagac |
| 1921 | atcagcaacg gggcactggc tttcaccccc ttggacacag ctgacccagc |
| | caagtcccca |
| 1981 | aagcccagca atggggctgg ggtccccaat ggagccctcc gggagtccgg |
| | gggctcaggc |
| 2041 | ttccggtctc cccacctgtg gaagaagtcc agcacgctga ccagcagccg |
| | cctagccacc |
| 2101 | ggcgaggagg agggcggtgg cagctccagc aagcgcttcc tgcgctcttg |
| | ctccgcctcc |
| 2161 | tgcgttcccc atggggccaa ggacacggag tggaggtcag tcacgctgcc |
| | tcgggacttg |
| 2221 | cagtccacgg gaagacagtt tgactcgtcc acatttggag ggcacaaaag |
| | tgagaagccg |
| 2281 | gctctgcctc ggaagagggc aggggagaac aggtctgacc aggtgacccg |
| | aggcacagta |
| 2341 | acgcctcccc ccaggctggt gaaaaagaat gaggaagctg ctgatgaggt |
| | cttcaaagac |
| 2401 | atcatggagt ccagcccggg ctccagcccg cccaacctga ctccaaaacc |
| | cctccggcgg |
| 2461 | caggtcaccg tggcccctgc ctcgggcctc ccccacaagg aagaagctgg |
| | aaagggcagt |
| 2521 | gccttaggga cccctgctgc agctgagcca gtgaccccca ccagcaaagc |
| | aggctcaggt |
| 2581 | gcaccagggg gcaccagcaa gggccccgcc gaggagtcca gagtgaggag |
| | gcacaagcac |
| 2641 | tcctctgagt cgccagggag ggacaagggg aaattgtcca ggctcaaacc |
| | tgccccgccg |
| 2701 | cccccaccag cagcctctgc agggaaggct ggaggaaagc cctcgcagag |
| | cccgagccag |
| 2761 | gaggcggccg gggaggcagt cctgggcgca aagacaaaag ccacgagtct |
| | ggttgatgct |
| 2821 | gtgaacagtg acgctgccaa gcccagccag ccgggagagg gcctcaaaaa |
| | gcccgtgctc |
| 2881 | ccggccactc caaagccaca gtccgccaag ccgtcgggga cccccatcag |
| | cccagccccc |
| 2941 | gttccctcca cgttgccatc agcatcctcg gccctggcag gggaccagcc |
| | gtcttccacc |
| 3001 | gccttcatcc ctctcatatc aacccgagtg tctcttcgga aaacccgcca |
| | gcctccagag |
| 3061 | cggatcgcca gcggcgccat caccaagggc gtggtcctgg acagcaccga |
| | ggcgctgtgc |
| 3121 | ctcgccatct ctaggaactc cgagcagatg gccagccaca gcgcagtgct |
| | ggaggccggc |
| 3181 | aaaaacctct acacgttctg cgtgagctat gtggattcca tccagcaaat |
| | gaggaacaag |
| 3241 | tttgccttcc gagaggccat caacaaactg gagaataatc tccgggagct |
| | tcagatctgc |
| 3301 | ccggcgacag caggcagtgg tccagcggcc actcaggact tcagcaagct |
| | cctcagttcg |
| 3361 | gtgaaggaaa tcagtgacat agtgcagagg tag |

ABL1 Protein (Homo sapiens)

| SEQ ID NO: 32 | |
|---|---|
| 1 | mleiclklvg ckskkglsss sscyleealq rpvasdfepq glseaarwns |
| | kenllagpse |
| 61 | ndpnlfvaly dfvasgdntl sitkgeklrv lgynhngewc eaqtkngqgw |
| | vpsnyitpvn |
| 121 | slekhswyhg pvsrnaaeyl lssgingsfl vresesspgq rsislryegr |
| | vyhyrintas |
| 181 | dgklyvsses rfntlaelvh hhstvadgli ttlhypapkr nkptvygvsp |
| | nydkwemert |
| 241 | ditmkhklgg gqygevyegv wkkysltvav ktlkedtmev eeflkeaavm |
| | keikhpnlvq |
| 301 | llgvctrepp fyiitefmty gnlldylrec nrqevnavvl lymatqissa |
| | meylekknfi |
| 361 | hrdlaarncl vgenhlvkva dfglsrlmtg dtytahagak fpikwtapes |
| | laynkfsiks |
| 421 | dvwafgvllw eiatygmspy pgidlsqvye llekdyrmer pegcpekvye |
| | lmracwqwnp |
| 481 | sdrpsfaeih qafetmfqes sisdevekel gkqgvrgavs tllqapelpt |
| | ktrtsrraae |
| 541 | hrdttdvpem phskgqgesd pldhepavsp llprkergpp egglnederl |
| | lpkdkktnlf |
| 601 | salikkkkkt aptppkrsss fremdgqper rgageeegrd isngalaftp |
| | ldtadpaksp |
| 661 | kpsngagvpn galresggsg frsphlwkks stltssrlat geeegggsss |
| | krflrscsas |
| 721 | cvphgakdte wrsvtlprdl qstgrqfdss tfgghksekp alprkragen |
| | rsdqvtrgtv |
| 781 | tppprlvkkn eeaadevfkd imesspgssp pnltpkplrr qvtvapasgl |
| | phkeeagkgs |
| 841 | algtpaaaep vtptskagsg apggtskgpa eesrvrrhkh ssespgrdkg |
| | klsrlkpapp |
| 901 | pppaasagka ggkpsqspsq eaageavlga ktkatslvda vnsdaakpsq |
| | pgeglkkpvl |
| 961 | patpkpqsak psgtpispap vpstlpsass alagdqpsst afiplistrv |
| | slrktrqppe |
| 1021 | riasgaitkg vvldstealc laisrnseqm ashsavleag knlytfcvsy |
| | vdsiqqmrnk |
| 1081 | fafreainkl ennlrelqic patagsgpaa tqdfskllss vkeisdivqr |

Nup214 cDNA (Homo sapiens)

| SEQ ID NO: 33 | |
|---|---|
| 1 | atgggagacg agatggatgc catgattccc gagcgggaga tgaaggattt |
| | tcagtttaga |
| 61 | gcgctaaaga aggtgagaat ctttgactcc cctgaggaat tgcccaagga |
| | acgctcgagt |
| 121 | ctgcttgctg tgtccaacaa atatggtctg gtcttcgctg gtggagccag |
| | tggcttgcag |
| 181 | atttttccta ctaaaaatct tcttattcaa aataaacccg gagatgatcc |
| | caacaaaata |
| 241 | gttgataaag tccaaggctt gctagttcct atgaaattcc caatccatca |
| | cctggccttg |
| 301 | agctgtgata acctcacact ctctgcgtgc atgatgtcca gtgaatatgg |
| | ttccattatt |
| 361 | gctttttttg atgttcgcac attctcaaat gaggctaaac agcaaaaacg |
| | cccatttgcc |
| 421 | tatcataagc ttttgaaaga tgcaggaggc atggtgattg atatgaagtg |
| | gaaccccact |
| 481 | gtcccctcca tggtggcagt ttgtctggct gatggtagta ttgctgtcct |
| | gcaagtcacg |
| 541 | gaaacagtga aagtatgtgc aactcttcct tccacggtag cagtaacctc |
| | tgtgtgctgg |
| 601 | agccccaaag gaaagcagct ggcagtggga aaacagaatg gaactgtggt |
| | ccagtatctt |
| 661 | cctactttgc aggaaaaaaa agtcattcct tgtcctccgt tttatgagtc |
| | agatcatcct |
| 721 | gtcagagttc tggatgtgct gtggattggt acctacgtct tcgccatagt |
| | gtatgctgct |
| 781 | gcagatggga ccctggaaac gtctccagat gtggtgatgg ctctactacc |
| | gaaaaaagaa |
| 841 | gaaaagcacc cagagatatt tgtgaacttt atggagccct gttatggcag |
| | ctgcacggag |
| 901 | agacagcatc attactacct cagttacatt gaggaatggg atttagtgct |
| | ggcagcatct |
| 961 | gcggcttcaa cagaagttag tatccttgct cgacaaagtg atcagattaa |
| | ttgggaatct |
| 1021 | tggctactgg aggattctag tcgagctgaa ttgcctgtga cagacaagag |
| | tgatgactcc |
| 1081 | ttgcccatgg gagttgtcgt agactataca aaccaagtgg aaatcaccat |
| | cagtgatgaa |
| 1141 | aagactcttc ctcctgctcc agttctcatg ttactttcaa cagatggtgt |
| | gctttgtcca |
| 1201 | ttttatatga ttaatcaaaa tcctggggtt aagtctctca tcaaaacacc |
| | agagcgactt |
| 1261 | tcattagaag gagagcgaca gcccaagtca ccaggaagta ctcccactac |
| | cccaacctcc |
| 1321 | tctcaagccc cacagaaact ggatgcttct gcagctgcag cccctgcctc |
| | tctgccacct |
| 1381 | tcatcacctg ctgctcccat tgccactttt tctttgcttc ctgctggtgg |
| | agcccccact |
| 1441 | gtgttctcct ttggttcttc atctttgaag tcatctgcta cggtcactgg |
| | ggagccccct |
| 1501 | tcatattcca gtggctccga cagctccaaa gcagccccag gccctggccc |
| | atcaaccttc |
| 1561 | tcttttgttc ccccttctaa agcctcccta gcccccaccc ctgcagcgtc |
| | tcctgtggct |
| 1621 | ccatcagctg cttcattctc ctttggatca tctggtttta agcctaccct |
| | ggaaagcaca |
| 1681 | ccagtgccaa gtgtgtctgc tccaaatata gcaatgaagc cctccttccc |
| | accctcaacc |
| 1741 | tctgctgtca aagtcaacct tagtgaaaag tttactgctg cagctacctc |
| | tactcctgtt |
| 1801 | agtagctccc agagcgcacc cccgatgtcg ccattctctt ctgcctccaa |
| | gccagctgct |
| 1861 | tctggaccac tcagccaccc cacacctctc tcagcaccac ctagttccgt |
| | gccattgaag |
| 1921 | tcctcagtct tgccctcacc atcaggacga tctgctcagg gcagttcaag |
| | cccagtgccc |
| 1981 | tcaatggtac agaaatcacc caggataacc cctccagcgg caaagccagg |
| | ctctccccag |
| 2041 | gcaaagtcac ttcagcctgc tgttgcagaa aagcagggac atcagtggaa |
| | agattcagat |
| 2101 | cctgtaatgg ctggaattgg ggaggagatt gcacactttc agaaggagtt |
| | ggaagagtta |
| 2161 | aaagcccgaa cttccaaagc ctgtttccaa gtgggcactt ctgaggagat |
| | gaagatgctg |
| 2221 | cgaacagaat cagatgactt gcataccttt cttttggaga ttaaagagac |
| | cacagagtcg |
| 2281 | cttcatggag atataagtag cctgaaaaca actttacttg agggctttgc |
| | tggtgttgag |
| 2341 | gaagccagag aacaaaatga aagaaatcgt gactctggtt atctgcattt |
| | gctttataaa |
| 2401 | agaccactgg atcccaagag tgaagctcag cttcaggaaa ttcggcgcct |
| | tcatcagtat |
| 2461 | gtgaaatttg ctgtccaaga tgtgaatgat gttctagact tggagtggga |
| | tcagcatctg |
| 2521 | gaacaaaaga aaaaacaaag gcacctgctt gtgccagagc gagagacact |
| | gtttaacacc |
| 2581 | ctagccaaca atcgggaaat catcaaccaa cagaggaaga ggctgaatca |
| | cctggtggat |
| 2641 | agtcttcagc agctccgcct ttacaaacag acttccctgt ggagcctgtc |
| | ctcggctgtt |
| 2701 | ccttcccaga gcagcattca cagttttgac agtgacctgg aaagcctgtg |
| | caatgctttg |
| 2761 | ttgaaaacca ccatagaatc tcacaccaaa tccttgccca aagtaccagc |
| | caaactgtcc |
| 2821 | cccatgaaac aggcacaact gagaaacttc ttggccaaga ggaagacccc |
| | accagtgaga |
| 2881 | tccactgctc cagccagcct gtctcgatca gcctttctgt ctcagagata |
| | ttatgaagac |
| 2941 | ttggatgaag tcagctcaac gtcatctgtc tcccagtctc tggagagtga |
| | agatgcacgg |
| 3001 | acgtcctgta aagatgacga ggcagtggtt caggcccctc ggcacgcccc |
| | cgtggttcgc |
| 3061 | actccttcca tccagcccag tctcttgccc catgcagcac cttttgctaa |
| | atctcacctg |
| 3121 | gttcatggtt cttcacctgg tgtgatggga acttcagtgg ctacatctgc |
| | tagcaaaatt |
| 3181 | attcctcaag gggccgatag cacaatgctt gccacgaaaa ccgtgaaaca |
| | tggtgcacct |
| 3241 | agtccttccc accccatctc agccccgcag gcagctgccg cagcagcact |
| | caggcggcag |
| 3301 | atggccagtc aggcaccagc tgtaaacact ttgactgaat caacgttgaa |
| | gaatgtccct |
| 3361 | caagtggtaa atgtgcagga attgaagaat aaccctgcaa ccccttctac |
| | agccatgggt |
| 3421 | tcttcagtgc cctactccac agccaaaaca cctcacccag tgttgacccc |
| | agtggctgct |
| 3481 | aaccaagcca agcaggggtc tctaataaat tcccttaagc catctgggcc |
| | tacaccagca |
| 3541 | tccggtcagt tatcatctgg tgacaaagct tcagggacag ccaagataga |
| | aacagctgtg |
| 3601 | acttcaaccc catctgcttc tgggcagttc agcaagcctt tctcattttc |
| | tccatcaggg |
| 3661 | actggcttta attttgggat aatcacacca acaccgtctt ctaatttcac |
| | tgctgcacaa |
| 3721 | ggggcaacac cctccactaa agagtcaagc cagccggacg cattctcatc |
| | tggtggggga |
| 3781 | agcaaacctt cttatgaggc cattcctgaa agctcacctc cctcaggaat |
| | cacatccgca |
| 3841 | tcaaacacca ccccaggaga acctgccgca tctagcagca gacctgtggc |
| | accttctgga |
| 3901 | actgctcttt ccaccacctc tagtaagctg gaaaccccac cgtccaagct |
| | gggagagctt |
| 3961 | ctgtttccaa gttctttggc tggagagact ctgggaagtt tttcaggact |
| | gcgggttggc |
| 4021 | caagcagatg attctacaaa accaaccaat aaggcttcat ccacaagcct |
| | aactagtacc |
| 4081 | cagccaacca agacgtcagg cgtgccctca gggtttaatt ttactgcccc |
| | cccggtgtta |
| 4141 | gggaagcaca cggagccccc tgtgacatcc tctgcaacca ccacctcagt |
| | agcaccacca |
| 4201 | gcagccacca gcacttcctc aactgccgtt tttggcagtc tgccagtcac |
| | cagtgcagga |
| 4261 | tcctctgggg tcatcagttt tggtgggaca tctctaagtg ctggcaagac |
| | tagtttttca |
| 4321 | tttggaagcc aacagaccaa tagcacagtg cccccatctg ccccaccacc |
| | aactacagct |
| 4381 | gccactcccc ttccaacatc attccccaca ttgtcatttg gtagcctcct |
| | gagttcagca |
| 4441 | actaccccct ccctgcctat gtccgctggc agaagcacag aagaggccac |
| | ttcatcagct |
| 4501 | ttgcctgaga agccaggtga cagtgaggtc tcagcatcag cagcctcact |
| | tctagaggag |
| 4561 | caacagtcag cccagcttcc ccaggctcct ccgcaaactt ctgactctgt |
| | taaaaaagaa |
| 4621 | cctgttcttg cccagcctgc agtcagcaac tctggcactg cagcatctag |
| | tactagtctt |
| 4681 | gtagcacttt ctgcagaggc taccccagcc accacggggg tccctgatgc |
| | caggacggag |
| 4741 | gcagtaccac ctgcttcctc cttttctgtg cctgggcaga ctgctgtcac |
| | agcagctgct |
| 4801 | atctcaagtg caggccctgt ggccgtcgaa acatcaagta cccccatagc |
| | ctccagcacc |
| 4861 | acgtccattg ttgctcccgg cccatctgca gaggcagcag catttggtac |
| | cgtcacttct |
| 4921 | ggctcatccg tctttgctca gcctcctgct gccagttcta gctcagcttt |
| | caaccagctc |
| 4981 | accaacaaca cagccactgc cccctctgcc acgcccgtgt ttgggcaagt |
| | ggcagccagc |
| 5041 | accgcaccaa gtctgtttgg gcagcagact ggtagcacag ccagcacagc |
| | agctgccaca |
| 5101 | ccacaggtca gcagctcagg gtttagcagc ccagcttttg gtaccacagc |
| | cccaggggtc |
| 5161 | tttggacaga caaccttcgg gcaggcctca gtctttgggc agtcggcgag |
| | cagtgctgca |
| 5221 | agtgtctttt ccttcagtca gcctgggttc agttccgtgc ctgccttcgg |
| | tcagcctgct |
| 5281 | tcctccactc ccacatccac cagtggaagt gtctttggtg ccgcctcaag |
| | taccagtagc |
| 5341 | tccagttcct tctcatttgg acagtcttct cccaacacag gaggggggct |
| | gtttggccaa |
| 5401 | agcaacgctc ctgcttttgg gcagagtcct ggctttggac agggaggctc |
| | tgtctttggt |
| 5461 | ggtacctcag ctgccaccac aacagcagca acctctgggt tcagcttttg |
| | ccaagcttca |
| 5521 | ggttttgggt ctagtaatac tggttctgtg tttggtcaag cagccagtac |
| | tggtggaata |
| 5581 | gtctttggcc agcaatcatc ctcttccagt ggtagcgtgt ttgggtctgg |
| | aaacactgga |
| 5641 | agagggggag gtttcttcag tggccttgga ggaaaaccca gtcaggatgc |
| | agccaacaaa |
| 5701 | aacccattca gctcggccag tgggggcttt ggatccacag ctacctcaaa |
| | tacctctaac |
| 5761 | ctatttggaa acagtggggc caagacattt ggtggatttg ccagctcgtc |
| | gtttggagag |
| 5821 | cagaaaccca ctggcacttt cagctctgga ggaggaagtg tggcatccca |
| | aggctttggg |
| 5881 | ttttcctctc caaacaaaac aggtggcttc ggtgctgctc cagtgtttgg |
| | cagccctcct |
| 5941 | acttttgggg gatcccctgg gtttggaggg gtgccagcat tcggttcagc |
| | cccagccttt |
| 6001 | acaagccctc tgggctcgac gggaggcaaa gtgttcggag agggcactgc |
| | agctgccagc |
| 6061 | gcaggaggat tcgggtttgg gagcagcagc aacaccacat ccttcggcac |
| | gctcgcgagt |
| 6121 | cagaatgccc ccactttcgg atcactgtcc caacagactt ctggttttgg |
| | gacccagagt |
| 6181 | agcggattct ctggttttgg atcaggcaca ggagggttca gctttgggtc |
| | aaataactcg |
| 6241 | tctgtccagg gttttggtgg ctggcgaagc tga |

NUP214 Protein (Homo sapiens)

| SEQ ID NO: 34 | |
|---|---|
| 1 | mgdemdamip eremkdfqfr alkkvrifds peelpkerss llavsnkygl |
| | vfaggasglq |
| 61 | ifptknlliq nkpgddpnki vdkvqgllvp mkfpihhlal scdnltlsac |
| | mmsseygsii |
| 121 | affdvrtfsn eakqqkrpfa yhkllkdagg mvidmkwnpt vpsmvavcla |
| | dgsiavlqvt |
| 181 | etvkvcatlp stvavtsvcw spkgkqlavg kqngtvvqyl ptlqekkvip |
| | cppfyesdhp |
| 241 | vrvldvlwig tyvfaivyaa adgtletspd vvmallpkke ekhpeifvnf |
| | mepcygscte |
| 301 | rqhhyylsyi eewdlvlaas aastevsila rqsdqinwes wlledssrae |
| | lpvtdksdds |
| 361 | lpmgvvvdyt nqveitisde ktlppapvlm llstdgvlcp fyminqnpgv |
| | ksliktperl |
| 421 | slegerqpks pgstpttpts sqapqkldas aaaapaslpp sspaapiatf |
| | sllpaggapt |
| 481 | vfsfgssslk ssatvtgepp syssgsdssk aapgpgpstf sfvppskasl |
| | aptpaaspva |
| 541 | psaasfsfgs sgfkptlest pvpsvsapni amkpsfppst savkvnlsek |
| | ftaaatstpv |
| 601 | sssqsappms pfssaskpaa sgplshptpl sappssvplk ssvlpspsgr |
| | saqgssspvp |
| 661 | smvqksprit ppaakpgspq akslqpavae kqghqwkdsd pvmagigeei |
| | ahfqkeleel |
| 721 | kartskacfq vgtseemkml rtesddlhtf lleikettes lhgdisslkt |
| | tllegfagve |
| 781 | eareqnernr dsgylhllyk rpldpkseaq lqeirrlhqy vkfavqdvnd |
| | vldlewdqhl |
| 841 | eqkkkqrhll vperetlfnt lannreiinq qrkrlnhlvd slqqlrlykq |
| | tslwslssav |
| 901 | psqssihsfd sdleslcnal lkttieshtk slpkvpakls pmkqaqlrnf |
| | lakrktppvr |
| 961 | stapaslsrs aflsqryyed ldevsstssv sqslesedar tsckddeavv |
| | qaprhapvvr |
| 1021 | tpsiqpsllp haapfakshl vhgsspgvmg tsvatsaski ipqgadstml |
| | atktvkhgap |
| 1081 | spshpisapq aaaaaalrrq masqapavnt ltestlknvp qvvnvqelkn |
| | npatpstamg |
| 1141 | ssvpystakt phpvltpvaa nqakqgslin slkpsgptpa sgqlssgdka |
| | sgtakietav |
| 1201 | tstpsasgqf skpfsfspsg tgfnfgiitp tpssnftaaq gatpstkess |
| | qpdafssggg |
| 1261 | skpsyeaipe ssppsgitsa snttpgepaa sssrpvapsg talsttsskl |
| | etppsklgel |
| 1321 | lfpsslaget lgsfsglrvg qaddstkptn kasstsltst qptktsgvps |
| | gfnftappvl |
| 1381 | gkhteppvts satttsvapp aatstsstav fgslpvtsag ssgvisfggt |
| | slsagktsfs |
| 1441 | fgsqqtnstv ppsappptta atplptsfpt lsfgsllssa ttpslpmsag |
| | rsteeatssa |
| 1501 | lpekpgdsev sasaasllee qqsaqlpqap pqtsdsvkke pvlaqpavsn |
| | sgtaasstsl |
| 1561 | valsaeatpa ttgvpdarte avppassfsv pgqtavtaaa issagpvave |
| | tsstpiasst |
| 1621 | tsivapgpsa eaaafgtvts gssvfaqppa assssafnql tnntatapsa |
| | tpvfgqvaas |
| 1681 | tapslfgqqt gstastaaat pqvsssgfss pafgttapgv fgqttfgqas |
| | vfgqsassaa |
| 1741 | svfsfsqpgf ssvpafgqpa sstptstsgs vfgaasstss sssfsfgqss |
| | pntggglfgq |
| 1801 | snapafgqsp gfgqggsvfg gtsaatttaa tsgfsfcqas gfgssntgsv |
| | fgqaastggi |
| 1861 | vfgqqsssss gsvfgsgntg rgggffsglg gkpsqdaank npfssasggf |
| | gstatsntsn |
| 1921 | lfgnsgaktf ggfasssfge qkptgtfssg ggsvasqgfg fsspnktggf |
| | gaapvfgspp |
| 1981 | tfggspgfgg vpafgsapaf tsplgstggk vfgegtaaas aggfgfgsss |
| | nttsfgtlas |
| 2041 | qnaptfgsls qqtsgfgtqs sgfsgfgsgt ggfsfgsnns svqgfggwrs |

Trp53 cDNA (Homo sapiens)

| SEQ ID NO: 35 | |
|---|---|
| 1 | atggaggagc cgcagtcaga tcctagcgtc gagccccctc tgagtcagga |
| | aacattttca |
| 61 | gacctatgga aactacttcc tgaaaacaac gttctgtccc ccttgccgtc |
| | ccaagcaatg |
| 121 | gatgatttga tgctgtcccc ggacgatatt gaacaatggt tcactgaaga |
| | cccaggtcca |
| 181 | gatgaagctc ccagaatgcc agaggctgct ccccccgtgg cccctgcacc |
| | agcagctcct |
| 241 | acaccggcgg cccctgcacc agccccctcc tggcccctgt catcttctgt |
| | cccttcccag |
| 301 | aaaacctacc agggcagcta cggtttccgt ctgggcttct tgcattctgg |
| | gacagccaag |
| 361 | tctgtgactt gcacgtactc ccctgccctc aacaagatgt tttgccaact |
| | ggccaagacc |
| 421 | tgccctgtgc agctgtgggt tgattccaca cccccgcccg gcacccgcgt |
| | ccgcgccatg |
| 481 | gccatctaca agcagtcaca gcacatgacg gaggttgtga ggcgctgccc |
| | ccaccatgag |
| 541 | cgctgctcag atagcgatgg tctggcccct cctcagcatc ttatccgagt |
| | ggaaggaaat |
| 601 | ttgcgtgtgg agtatttgga tgacagaaac acttttcgac atagtgtggt |
| | ggtgccctat |
| 661 | gagccgcctg aggttggctc tgactgtacc accatccact acaactacat |
| | gtgtaacagt |
| 721 | tcctgcatgg gcggcatgaa ccggaggccc atcctcacca tcatcacact |
| | ggaagactcc |
| 781 | agtggtaatc tactgggacg gaacagcttt gaggtgcgtg tttgtgcctg |
| | tcctgggaga |
| 841 | gaccggcgca cagaggaaga gaatctccgc aagaaagggg agcctcacca |
| | cgagctgccc |
| 901 | ccagggagca ctaagcgagc actgcccaac aacaccagct cctctcccca |
| | gccaaagaag |
| 961 | aaaccactgg atggagaata tttcaccctt cagatccgtg ggcgtgagcg |
| | cttcgagatg |
| 1021 | ttccgagagc tgaatgaggc cttggaactc aaggatgccc aggctgggaa |
| | ggagccaggg |
| 1081 | gggagcaggg ctcactccag ccacctgaag tccaaaaagg gtcagtctac |
| | ctcccgccat |
| 1141 | aaaaaactca tgttcaagac agaagggcct gactcagact ga |

TRP53 Protein (Homo sapiens)

| SEQ ID NO: 36 | |
|---|---|
| 1 | meepqsdpsv epplsqetfs dlwkllpenn vlsplpsqam ddlmlspddi |
| | eqwftedpgp |
| 61 | deaprmpeaa ppvapapaap tpaapapaps wplsssvpsq ktyqgsygfr |
| | lgflhsgtak |
| 121 | svtctyspal nkmfcqlakt cpvqlwvdst pppgtrvram aiykqsqhmt |
| | evvrrcphhe |
| 181 | rcsdsdglap pqhlirvegn lrveylddrn tfrhsvvvpy eppevgsdct |
| | tihynymcns |
| 241 | scmggmnrrp iltiitleds sgnllgrnsf evrvcacpgr drrteeenlr |
| | kkgephhelp |
| 301 | pgstkralpn ntssspqpkk kpldgeyftl qirgrerfem frelnealel |
| | kdaqagkepg |
| 361 | gsrahsshlk skkgqstsrh kklmfktegp dsd |

Bcl6 cDNA (Homo sapiens)

| SEQ ID NO: 37 | |
|---|---|
| 1 | atggcctcgc cggctgacag ctgtatccag ttcacccgcc atgccagtga |
| | tgttcttctc |
| 61 | aaccttaatc gtctccggag tcgagacatc ttgactgatg ttgtcattgt |
| | tgtgagccgt |
| 121 | gagcagttta gagcccataa aacggtcctc atggcctgca gtggcctgtt |
| | ctatagcatc |
| 181 | tttacagacc agttgaaatg caaccttagt gtgatcaatc tagatcctga |
| | gatcaaccct |
| 241 | gagggattct gcatcctcct ggacttcatg tacacatctc ggctcaattt |
| | gcgggagggc |
| 301 | aacatcatgg ctgtgatggc cacggctatg tacctgcaga tggagcatgt |
| | tgtggacact |
| 361 | tgccggaagt ttattaaggc cagtgaagca gagatggttt ctgccatcaa |
| | gcctcctcgt |
| 421 | gaagagttcc tcaacagccg gatgctgatg ccccaagaca tcatggccta |
| | tcggggtcgt |
| 481 | gaggtggtgg agaacaacct gccactgagg agcgcccctg ggtgtgagag |
| | cagagccttt |
| 541 | gcccccagcc tgtacagtgg cctgtccaca ccgccagcct cttattccat |
| | gtacagccac |
| 601 | ctccctgtca gcagcctcct cttctccgat gaggagtttc gggatgtccg |
| | gatgcctgtg |
| 661 | gccaacccct tccccaagga gcgggcactc ccatgtgata gtgccaggcc |
| | agtccctggt |
| 721 | gagtacagcc ggccgacttt ggaggtgtcc cccaatgtgt gccacagcaa |
| | tatctattca |
| 781 | cccaaggaaa caatcccaga agaggcacga agtgatatgc actacagtgt |
| | ggctgagggc |
| 841 | ctcaaacctg ctgccccctc agcccgaaat gccccctact tcccttgtga |
| | caaggccagc |
| 901 | aaagaagaag agagaccctc ctcggaagat gagattgccc tgcatttcga |
| | gccccccaat |
| 961 | gcacccctga accggaaggg tctggttagt ccacagagcc cccagaaatc |
| | tgactgccag |
| 1021 | cccaactcgc ccacagagtc ctgcagcagt aagaatgcct gcatcctcca |
| | ggcttctggc |
| 1081 | tcccctccag ccaagagccc cactgacccc aaagcctgca actggaagaa |
| | atacaagttc |
| 1141 | atcgtgctca acagcctcaa ccagaatgcc aaaccagagg ggcctgagca |
| | ggctgagctg |
| 1201 | ggccgccttt ccccacgagc ctacacggcc ccacctgcct gccagccacc |
| | catggagcct |
| 1261 | gagaaccttg acctccagtc cccaaccaag ctgagtgcca gcggggagga |
| | ctccaccatc |
| 1321 | ccacaagcca gccggctcaa taacatcgtt aacaggtcca tgacgggctc |
| | tccccgcagc |
| 1381 | agcagcgaga gccactcacc actctacatg caccccccga agtgcacgtc |
| | ctgcggctct |
| 1441 | cagtccccac agcatgcaga gatgtgcctc cacaccgctg gccccacgtt |
| | ccctgaggag |
| 1501 | atgggagaga cccagtctga gtactcagat tctagctgtg agaacggggc |
| | cttcttctgc |
| 1561 | aatgagtgtg actgccgctt ctctgaggag gcctcactca agaggcacac |
| | gctgcagacc |
| 1621 | cacagtgaca aaccctacaa gtgtgaccgc tgccaggcct ccttccgcta |
| | caagggcaac |
| 1681 | ctcgccagcc acaagaccgt ccataccggt gagaaaccct atcgttgcaa |
| | catctgtggg |
| 1741 | gcccagttca accggccagc caacctgaaa acccacactc gaattcactc |
| | tggagagaag |
| 1801 | ccctacaaat gcgaaacctg cggagccaga tttgtacagg tggcccacct |
| | ccgtgcccat |
| 1861 | gtgcttatcc acactggtga gaagccctat ccctgtgaaa tctgtggcac |
| | ccgtttccgg |
| 1921 | caccttcaga ctctgaagag ccacctgcga atccacacag gagagaaacc |
| | ttaccattgt |
| 1981 | gagaagtgta acctgcattt ccgtcacaaa agccagctgc gacttcactt |
| | gcgccagaag |
| 2041 | catggcgcca tcaccaacac caaggtgcaa taccgcgtgt cagccactga |
| | cctgcctccg |
| 2101 | gagctcccca aagcctgctg a |

BCL6 Protein (Homo sapiens)

| SEQ ID NO: 38 | |
|---|---|
| 1 | maspadsciq ftrhasdvll nlnrlrsrdi ltdvvivvsr eqfrahktvl |
| | macsglfysi |
| 61 | ftdqlkcnls vinldpeinp egfcilldfm ytsrlnlreg nimavmatam |
| | ylqmehvvdt |
| 121 | crkfikasea emvsaikppr eeflnsrmlm pqdimayrgr evvennlplr |
| | sapgcesraf |
| 181 | apslysglst ppasysmysh lpvssllfsd eefrdvrmpv anpfpkeral |
| | pcdsarpvpg |
| 241 | eysrptlevs pnvchsniys pketipeear sdmhysvaeg lkpaapsarn |
| | apyfpcdkas |
| 301 | keeerpssed eialhfeppn aplnrkglvs pqspqksdcq pnsptescss |
| | knacilqasg |
| 361 | sppaksptdp kacnwkkykf ivlnslnqna kpegpeqael grlsprayta |
| | ppacqppmep |
| 421 | enldlqsptk lsasgedsti pqasrlnniv nrsmtgsprs sseshsplym |
| | hppkctscgs |
| 481 | qspqhaemcl htagptfpee mgetqseysd sscengaffc necdcrfsee |
| | aslkrhtlqt |
| 541 | hsdkpykcdr cqasfrykgn lashktvhtg ekpyrcnicg aqfnrpanlk |
| | thtrihsgek |
| 601 | pykcetcgar fvqvahlrah vlihtgekpy pceicgtrfr hlqtlkshlr |
| | ihtgekpyhc |
| 661 | ekcnlhfrhk sqlrlhlrqk hgaitntkvq yrvsatdlpp elpkac |

Negr1 cDNA (Homo sapiens)

| SEQ ID NO: 39 | |
|---|---|
| 1 | atggacatga tgctgttggt gcagggtgct tgttgctcga accagtggct |
| | ggcggcggtg |
| 61 | ctcctcagcc tgtgctgcct gctaccctcc tgcctcccgg ctggacagag |
| | tgtggacttc |
| 121 | ccctgggcgg ccgtggacaa catgatggtc agaaaagggg acacggcggt |
| | gcttaggtgt |
| 181 | tatttggaag atggagcttc aaagggtgcc tggctgaacc ggtcaagtat |
| | tatttttgcg |
| 241 | ggaggtgata agtggtcagt ggatcctcga gtttcaattt caacattgaa |
| | taaaagggac |
| 301 | tacagcctcc agatacagaa tgtagatgtg acagatgatg gcccatacac |
| | gtgttctgtt |
| 361 | cagactcaac atacacccag aacaatgcag gtgcatctaa ctgtgcaagt |
| | tcctcctaag |
| 421 | atatatgaca tctcaaatga tatgaccgtc aatgaaggaa ccaacgtcac |
| | tcttacttgt |
| 481 | ttggccactg ggaaaccaga gccttccatt tcttggcgac acatctcccc |
| | atcagcaaaa |
| 541 | ccatttgaaa atggacaata tttggacatt tatggaatta caagggacca |
| | ggctggggaa |
| 601 | tatgaatgca gtgcggaaaa tgatgtgtca ttcccagatg tgaggaaagt |
| | aaaagttgtt |
| 661 | gtcaactttg ctcctactat tcaggaaatt aaatctggca ccgtgacccc |
| | cggacgcagt |
| 721 | ggcctgataa gatgtgaagg tgcaggtgtg ccgcctccag cctttgaatg |
| | gtacaaagga |
| 781 | gagaagaagc tcttcaatgg ccaacaagga attattattc aaaattttag |
| | cacaagatcc |
| 841 | attctcactg ttaccaacgt gacacaggag cacttcggca attatacctg |
| | tgtggctgcc |
| 901 | aacaagctag gcacaaccaa tgcgagcctg cctcttaacc ctccaagtac |
| | agcccagtat |
| 961 | ggaattaccg ggagcgctga tgttcttttc tcctgctggt accttgtgtt |
| | gacactgtcc |
| 1021 | tctttcacca gcatattcta cctgaagaat gccattctac aataa |

NEGR1 Protein (Homo sapiens)

SEQ ID NO: 40

| | |
|---|---|
| 1 | mdmmllvqga ccsnqwlaav llslccllps clpagqsvdf |
| | pwaavdnmmv rkgdtavlrc |
| 61 | yledgaskga wlnrssiifa ggdkwsvdpr vsistlnkrd |
| | yslqiqnvdv tddgpytcsv |
| 121 | qtqhtprtmq vhltvqvppk iydisndmtv negtnvtltc |
| | latgkpepsi swrhispsak |
| 181 | pfengqyldi ygitrdqage yecsaendvs fpdvrkvkvv |
| | vnfaptiqei ksgtvtpgrs |
| 241 | glircegagv pppafewykg ekklfngqqg iiiqnfstrs |
| | iltvtnvtqe hfgnytcvaa |
| 301 | nklgttnasl plnppstaqy gitgsadvlf scwylvltls |
| | sftsifylkn ailq |

Baalc cDNA (Homo sapiens)

SEQ ID NO: 41

| | |
|---|---|
| 1 | atgggctgcg gcgggagccg ggcggatgcc atcgagcccc |
| | gctactacga gagctggacc |
| 61 | cgggagacag aatccacctg gctcacctac accgactcgg |
| | acgcgccgcc cagcgccgcc |
| 121 | gccccggaca gcggccccga agcgggcggc ctgcactcgg |
| | gcatgctgga agatggactg |
| 181 | ccctccaatg gtgtgccccg atctacagcc ccaggtggaa |
| | tacccaaccc agagaagaag |
| 241 | acgaactgtg agacccagtg cccaaatccc cagagcctca |
| | gctcaggccc tctgacccag |
| 301 | aaacagaatg gccttcagac cacagaggct aaaagagatg |
| | ctaagagaat gcctgcaaaa |
| 361 | gaagtcacca ttaatgtaac agatagcatc caacagatgg |
| | acagaagtcg aagaatcaca |
| 421 | aagaactgtg tcaactag |

BAALC Protein (Homo sapiens)

SEQ ID NO: 42

| | |
|---|---|
| 1 | mgcggsrada iepryyeswt retestwlty tdsdappsaa |
| | apdsgpeagg lhsgmledgl |
| 61 | psngvprsta pggipnpekk tncetqcpnp qslssgpltq |
| | kqnglqttea krdakrmpak |
| 121 | evtinvtdsi qqmdrsrrit kncvn |

Fzd6 cDNA (Homo sapiens)

| SEQ ID NO: 43 | |
|---|---|
| 1 | atggaaatgt ttacattttt gttgacgtgt atttttctac ccctcctaag |
| | agggcacagt |
| 61 | ctcttcacct gtgaaccaat tactgttccc agatgtatga aaatggccta |
| | caacatgacg |
| 121 | tttttcccta atctgatggg tcattatgac cagagtattg ccgcggtgga |
| | aatggagcat |
| 181 | tttcttcctc tcgcaaatct ggaatgttca ccaaacattg aaactttcct |
| | ctgcaaagca |
| 241 | tttgtaccaa cctgcataga acaaattcat gtggttccac cttgtcgtaa |
| | actttgtgag |
| 301 | aaagtatatt ctgattgcaa aaaattaatt gacacttttg ggatccgatg |
| | gcctgaggag |
| 361 | cttgaatgtg acagattaca atactgtgat gagactgttc ctgtaacttt |
| | tgatccacac |
| 421 | acagaatttc ttggtcctca gaagaaaaca gaacaagtcc aaagagacat |
| | tggattttgg |
| 481 | tgtccaaggc atcttaagac ttctggggga caaggatata agtttctggg |
| | aattgaccag |
| 541 | tgtgcgcctc catgccccaa catgtatttt aaaagtgatg agctagagtt |
| | tgcaaaaagt |
| 601 | tttattggaa cagtttcaat attttgtctt tgtgcaactc tgttcacatt |
| | ccttactttt |
| 661 | ttaattgatg ttagaagatt cagataccca gagagaccaa ttatatatta |
| | ctctgtctgt |
| 721 | tacagcattg tatctcttat gtacttcatt ggatttttgc taggcgatag |
| | cacagcctgc |
| 781 | aataaggcag atgagaagct agaacttggt gacactgttg tcctaggctc |
| | tcaaaataag |
| 841 | gcttgcaccg ttttgttcat gcttttgtat tttttcacaa tggctggcac |
| | tgtgtggtgg |
| 901 | gtgattctta ccattacttg gttcttagct gcaggaagaa aatggagttg |
| | tgaagccatc |
| 961 | gagcaaaaag cagtgtggtt tcatgctgtt gcatggggaa caccaggttt |
| | cctgactgtt |
| 1021 | atgcttcttg ctatgaacaa agttgaagga gacaacatta gtggagtttg |
| | ctttgttggc |
| 1081 | ctttatgacc tggatgcttc tcgctacttt gtactcttgc cactgtgcct |
| | ttgtgtgttt |
| 1141 | gttgggctct ctcttctttt agctggcatt atttccttaa atcatgttcg |
| | acaagtcata |
| 1201 | caacatgatg gccggaacca agaaaaacta aagaaattta tgattcgaat |
| | tggagtcttc |
| 1261 | agcggcttgt atcttgtgcc attagtgaca cttctcggat gttacgtcta |
| | tgagcaagtg |
| 1321 | aacaggatta cctgggagat aacttgggtc tctgatcatt gtcgtcagta |
| | ccatatccca |
| 1381 | tgtccttatc aggcaaaagc aaaagctcga ccagaattgg ctttatttat |
| | gataaaatac |
| 1441 | ctgatgacat taattgttgg catctctgct gtcttctggg ttggaagcaa |
| | aaagacatgc |
| 1501 | acagaatggg ctgggttttt taaacgaaat cgcaagagag atccaatcag |
| | tgaaagtcga |
| 1561 | agagtactac aggaatcatg tgagtttttc ttaaagcaca attctaaagt |
| | taaacacaaa |
| 1621 | aagaagcact ataaaccaag ttcacacaag ctgaaggtca tttccaaatc |
| | catgggaacc |
| 1681 | agcacaggag ctacagcaaa tcatggcact tctgcagtag caattactag |
| | ccatgattac |
| 1741 | ctaggacaag aaactttgac agaaatccaa acctcaccag aaacatcaat |
| | gagagaggtg |
| 1801 | aaagcggacg gagctagcac ccccaggtta agagaacagg actgtggtga |
| | acctgcctcg |
| 1861 | ccagcagcat ccatctccag actctctggg gaacaggtcg acgggaaggg |
| | ccaggcaggc |
| 1921 | agtgtatctg aaagtgcgcg gagtgaagga aggattagtc caaagagtga |
| | tattactgac |
| 1981 | actggcctgg cacagagcaa caatttgcag gtccccagtt cttcagaacc |
| | aagcagcctc |
| 2041 | aaaggttcca catctctgct tgttcacccg gtttcaggag tgagaaaaga |
| | gcagggaggt |
| 2101 | ggttgtcatt cagatacttg a |

FZD6 Protein (Homo sapiens)

SEQ ID NO: 44

| | |
|---|---|
| 1 | memftflltc iflpllrghs lftcepitvp rcmkmaynmt |
| | ffpnlmghyd qsiaavemeh |
| 61 | flplanlecs pnietflcka fvptcieqih vvpperklce |
| | kvysdckkli dtfgirwpee |
| 121 | lecdrlqycd etvpvtfdph teflgpqkkt eqvqrdigfw |
| | cprhlktsgg qgykflgidq |
| 181 | cappcpnmyf ksdelefaks figtvsifcl catlftfltf |
| | lidvrrfryp erpiiyysvc |
| 241 | ysivslmyfi gfllgdstac nkadeklelg dtvvlgsqnk |
| | actvlfmlly fftmagtvww |
| 301 | viltitwfla agrkwsceai eqkavwfhav awgtpgfltv |
| | mllamnkveg dnisgvcfvg |
| 361 | lydldasryf vllplclcvf vglslllagi islnhvrqvi |
| | qhdgrnqekl kkfmirigvf |
| 421 | sglylvplvt llgcyvyeqv nritweitwv sdhcrqyhip |
| | cpyqakakar pelalfmiky |
| 481 | lmtlivgisa vfwvgskktc tewagffkrn rkrdpisesr |
| | rvlqesceff lkhnskvkhk |
| 541 | kkhykpsshk lkvisksmgt stgatanhgt savaitshdy |
| | lgqetlteiq tspetsmrev |
| 601 | kadgastprl reqdcgepas paasisrlsg eqvdgkgqag |
| | svsesarseg rispksditd |
| 661 | tglaqsnnlq vpsssepssl kgstsllvhp vsgvrkeqgg |
| | gchsdt |

Crebbp cDNA (Homo sapiens)

| SEQ ID NO: 45 | |
|---|---|
| 1 | atggctgaga acttgctgga cggaccgccc aaccccaaaa gagccaaact |
| | cagctcgccc |
| 61 | ggtttctcgg cgaatgacag cacagatttt ggatcattgt ttgacttgga |
| | aaatgatctt |
| 121 | cctgatgagc tgatacccaa tggaggagaa ttaggccttt taaacagtgg |
| | gaaccttgtt |
| 181 | ccagatgctg cttccaaaca taaacaactg tcggagcttc tacgaggagg |
| | cagcggctct |
| 241 | agtatcaacc caggaatagg aaatgtgagc gccagcagcc ccgtgcagca |
| | gggcctgggt |
| 301 | ggccaggctc aagggcagcc gaacagtgct aacatggcca gcctcagtgc |
| | catgggcaag |
| 361 | agccctctga gccagggaga ttcttcagcc cccagcctgc ctaaacaggc |
| | agccagcacc |
| 421 | tctgggccca cccccgctgc ctcccaagca ctgaatccgc aagcacaaaa |
| | gcaagtgggg |
| 481 | ctggcgacta gcagccctgc cacgtcacag actggacctg gtatctgcat |
| | gaatgctaac |
| 541 | tttaaccaga cccacccagg cctcctcaat agtaactctg gccatagctt |
| | aattaatcag |
| 601 | gcttcacaag ggcaggcgca agtcatgaat ggatctcttg gggctgctgg |
| | cagaggaagg |
| 661 | ggagctggaa tgccgtaccc tactccagcc atgcagggcg cctcgagcag |
| | cgtgctggct |
| 721 | gagaccctaa cgcaggtttc cccgcaaatg actggtcacg cgggactgaa |
| | caccgcacag |
| 781 | gcaggaggca tggccaagat gggaataact gggaacacaa gtccatttgg |
| | acagcccttt |
| 841 | agtcaagctg gagggcagcc aatgggagcc actggagtga acccccagtt |
| | agccagcaaa |
| 901 | cagagcatgg tcaacagttt gcccaccttc cctacagata tcaagaatac |
| | ttcagtcacc |
| 961 | aacgtgccaa atatgtctca gatgcaaaca tcagtgggaa ttgtacccac |
| | acaagcaatt |
| 1021 | gcaacaggcc ccactgcaga tcctgaaaaa cgcaaactga tacagcagca |
| | gctggttcta |
| 1081 | ctgcttcatg ctcataagtg tcagagacga gagcaagcaa acggagaggt |
| | tcgggcctgc |
| 1141 | tcgctcccgc attgtcgaac catgaaaaac gttttgaatc acatgacgca |
| | ttgtcaggct |
| 1201 | gggaaagcct gccaagttgc ccattgtgca tcttcacgac aaatcatctc |
| | tcattggaag |
| 1261 | aactgcacac gacatgactg tcctgtttgc ctccctttga aaaatgccag |
| | tgacaagcga |
| 1321 | aaccaacaaa ccatcctggg gtctccagct agtggaattc aaaacacaat |
| | tggttctgtt |
| 1381 | ggcacagggc aacagaatgc cacttcttta agtaacccaa atcccataga |
| | ccccagctcc |
| 1441 | atgcagcgag cctatgctgc tctcggactc ccctacatga accagcccca |
| | gacgcagctg |
| 1501 | cagcctcagg ttcctggcca gcaaccagca cagcctcaaa cccaccagca |
| | gatgaggact |
| 1561 | ctcaaccccc tgggaaataa tccaatgaac attccagcag gaggaataac |
| | aacagatcag |
| 1621 | cagcccccaa acttgatttc agaatcagct cttccgactt ccctgggggc |
| | cacaaaccca |
| 1681 | ctgatgaacg atggctccaa ctctggtaac attggaaccc tcagcactat |
| | accaacagca |
| 1741 | gctcctcctt ctagcaccgg tgtaaggaaa ggctggcacg aacatgtcac |
| | tcaggacctg |
| 1801 | cggagccatc tagtgcataa actcgtccaa gccatcttcc caacacctga |
| | tcccgcagct |
| 1861 | ctaaaggatc gccgcatgga aaacctggta gcctatgcta agaaagtgga |
| | aggggacatg |
| 1921 | tacgagtctg ccaacagcag ggatgaatat tatcacttat tagcagagaa |
| | aatctacaag |
| 1981 | atacaaaaag aactagaaga aaaacggagg tcgcgtttac ataaacaagg |
| | catcttgggg |
| 2041 | aaccagccag ccttaccagc cccgggggct cagccccctg tgattccaca |
| | ggcacaacct |
| 2101 | gtgagacctc caaatggacc cctgtccctg ccagtgaatc gcatgcaagt |
| | ttctcaaggg |
| 2161 | atgaattcat ttaaccccat gtccttgggg aacgtccagt tgccacaagc |
| | acccatggga |
| 2221 | cctcgtgcag cctccccaat gaaccactct gtccagatga acagcatggg |
| | ctcagtgcca |
| 2281 | gggatggcca tttctccttc ccgaatgcct cagcctccga acatgatggg |
| | tgcacacacc |
| 2341 | aacaacatga tggcccaggc gcccgctcag agccagtttc tgccacagaa |
| | ccagttcccg |
| 2401 | tcatccagcg gggcgatgag tgtgggcatg gggcagccgc cagcccaaac |
| | aggcgtgtca |
| 2461 | cagggacagg tgcctggtgc tgctcttcct aaccctctca acatgctggg |
| | gcctcaggcc |
| 2521 | agccagctac cttgccctcc agtgacacag tcaccactgc acccaacacc |
| | gcctcctgct |
| 2581 | tccacggctg ctggcatgcc atctctccag cacacgacac cacctgggat |
| | gactcctccc |
| 2641 | cagccagcag ctcccactca gccatcaact cctgtgtcgt cttccgggca |
| | gactcccacc |
| 2701 | ccgactcctg gctcagtgcc cagtgctacc caaacccaga gcacccctac |
| | agtccaggca |
| 2761 | gcagcccagg cccaggtgac cccgcagcct caaaccccag ttcagccccc |
| | gtctgtggct |
| 2821 | acccctcagt catcgcagca acagccgacg cctgtgcacg cccagcctcc |
| | tggcacaccg |
| 2881 | ctttcccagg cagcagccag cattgataac agagtcccta ccccctcctc |
| | ggtggccagc |
| 2941 | gcagaaacca attcccagca gccaggacct gacgtacctg tgctggaaat |
| | gaagacggag |
| 3001 | acccaagcag aggacactga gcccgatcct ggtgaatcca aaggggagcc |
| | caggtctgag |
| 3061 | atgatggagg aggatttgca aggagcttcc caagttaaag aagaaacaga |
| | catagcagag |
| 3121 | cagaaatcag aaccaatgga agtggatgaa aagaaacctg aagtgaaagt |
| | agaagttaaa |
| 3181 | gaggaagaag agagtagcag taacggcaca gcctctcagt caacatctcc |
| | ttcgcagccg |
| 3241 | cgcaaaaaaa tctttaaacc agaggagtta cgccaggccc tcatgccaac |
| | cctagaagca |
| 3301 | ctgtatcgac aggacccaga gtcattacct ttccggcagc ctgtagatcc |
| | ccagctcctc |
| 3361 | ggaattccag actattttga catcgtaaag aatcccatgg acctctccac |
| | catcaagcgg |
| 3421 | aagctggaca cagggcaata ccaagagccc tggcagtacg tggacgacgt |
| | ctggctcatg |
| 3481 | ttcaacaatg cctggctcta taatcgcaag acatcccgag tctataagtt |
| | ttgcagtaag |
| 3541 | cttgcagagg tctttgagca ggaaattgac cctgtcatgc agtcccttgg |
| | atattgctgt |
| 3601 | ggacgcaagt atgagttttc cccacagact ttgtgctgct atgggaagca |
| | gctgtgtacc |
| 3661 | attcctcgcg atgctgccta ctacagctat cagaataggt atcatttctg |
| | tgagaagtgt |
| 3721 | ttcacagaga tccagggcga gaatgtgacc ctgggtgacg acccttcaca |
| | gccccagacg |
| 3781 | acaatttcaa aggatcagtt tgaaaagaag aaaaatgata ccttagaccc |
| | cgaacctttc |
| 3841 | gttgattgca aggagtgtgg ccggaagatg catcagattt gcgttctgca |
| | ctatgacatc |
| 3901 | atttggcctt caggttttgt gtgcgacaac tgcttgaaga aaactggcag |
| | acctcgaaaa |
| 3961 | gaaaacaaat tcagtgctaa gaggctgcag accacaagac tgggaaacca |
| | cttggaagac |
| 4021 | cgagtgaaca aatttttgcg gcgccagaat caccctgaag ccggggaggt |
| | ttttgtccga |
| 4081 | gtggtggcca gctcagacaa gacggtggag gtcaagcccg ggatgaagtc |
| | acggtttgtg |
| 4141 | gattctgggg aaatgtctga atctttccca tatcgaacca aagctctgtt |
| | tgcttttgag |
| 4201 | gaaattgacg gcgtggatgt ctgctttttt ggaatgcacg tccaagaata |
| | cggctctgat |
| 4261 | tgcccccctc caaacacgag gcgtgtgtac atttcttatc tggatagtat |
| | tcatttcttc |
| 4321 | cggccacgtt gcctccgcac agccgtttac catgagatcc ttattggata |
| | tttagagtat |
| 4381 | gtgaagaaat tagggtatgt gacagggcac atctgggcct gtcctccaag |
| | tgaaggagat |
| 4441 | gattacatct tccattgcca cccacctgat caaaaaatac ccaagccaaa |
| | acgactgcag |
| 4501 | gagtggtaca aaaagatgct ggacaaggcg tttgcagagc ggatcatcca |
| | tgactacaag |
| 4561 | gatattttca aacaagcaac tgaagacagg ctcaccagtg ccaaggaact |
| | gccctatttt |
| 4621 | gaaggtgatt tctggcccaa tgtgttagaa gagagcatta aggaactaga |
| | acaagaagaa |
| 4681 | gaggagagga aaaaggaaga gagcactgca gccagtgaaa ccactgaggg |
| | cagtcagggc |
| 4741 | gacagcaaga atgccaagaa gaagaacaac aagaaaacca acaagaacaa |
| | aagcagcatc |
| 4801 | agccgcgcca acaagaagaa gcccagcatg cccaacgtgt ccaatgacct |
| | gtcccagaag |
| 4861 | ctgtatgcca ccatggagaa gcacaaggag gtcttcttcg tgatccacct |
| | gcacgctggg |
| 4921 | cctgtcatca acaccctgcc ccccatcgtc gaccccgacc ccctgctcag |
| | ctgtgacctc |
| 4981 | atggatgggc gcgacgcctt cctcaccctc gccagagaca agcactggga |
| | gttctcctcc |
| 5041 | ttgcgccgct ccaagtggtc cacgctctgc atgctggtgg agctgcacac |
| | ccagggccag |
| 5101 | gaccgctttg tctacacctg caacgagtgc aagcaccacg tggagacgcg |
| | ctggcactgc |
| 5161 | actgtgtgcg aggactacga cctctgcatc aactgctata acacgaagag |
| | ccatgcccat |
| 5221 | aagatggtga agtgggggct gggcctggat gacgagggca gcagccaggg |
| | cgagccacag |
| 5281 | tcaaagagcc cccaggagtc acgccggctg agcatccagc gctgcatcca |
| | gtcgctggtg |
| 5341 | cacgcgtgcc agtgccgcaa cgccaactgc tcgctgccat cctgccagaa |
| | gatgaagcgg |
| 5401 | gtggtgcagc acaccaaggg ctgcaaacgc aagaccaacg ggggctgccc |
| | ggtgtgcaag |
| 5461 | cagctcatcg ccctctgctg ctaccacgcc aagcactgcc aagaaaacaa |
| | atgccccgtg |
| 5521 | cccttctgcc tcaacatcaa acacaagctc cgccagcagc agatccagca |
| | ccgcctgcag |
| 5581 | caggcccagc tcatgcgccg gcggatggcc accatgaaca cccgcaacgt |
| | gcctcagcag |
| 5641 | agtctgcctt ctcctacctc agcaccgccc gggaccccca cacagcagcc |
| | cagcacaccc |
| 5701 | cagacgccgc agccccctgc ccagccccaa ccctcacccg tgagcatgtc |
| | accagctggc |
| 5761 | ttccccagcg tggcccggac tcagcccccc accacggtgt ccacagggaa |
| | gcctaccagc |
| 5821 | caggtgccgg cccccccacc cccggcccag ccccctcctg cagcggtgga |
| | agcggctcgg |
| 5881 | cagatcgagc gtgaggccca gcagcagcag cacctgtacc gggtgaacat |
| | caacaacagc |
| 5941 | atgcccccag gacgcacggg catggggacc ccggggagcc agatggcccc |
| | cgtgagcctg |
| 6001 | aatgtgcccc gacccaacca ggtgagcggg cccgtcatgc ccagcatgcc |
| | tcccgggcag |
| 6061 | tggcagcagg cgccccttcc ccagcagcag cccatgccag gcttgcccag |
| | gcctgtgata |
| 6121 | tccatgcagg cccaggcggc cgtggctggg ccccggatgc ccagcgtgca |
| | gccacccagg |
| 6181 | agcatctcac ccagcgctct gcaagacctg ctgcggaccc tgaagtcgcc |
| | cagctcccct |
| 6241 | cagcagcaac agcaggtgct gaacattctc aaatcaaacc cgcagctaat |
| | ggcagctttc |
| 6301 | atcaaacagc gcacagccaa gtacgtggcc aatcagcccg gcatgcagcc |
| | ccagcctggc |
| 6361 | ctccagtccc agcccggcat gcaaccccag cctggcatgc accagcagcc |
| | cagcctgcag |
| 6421 | aacctgaatg ccatgcaggc tggcgtgccg cggcccggtg tgcctccaca |
| | gcagcaggcg |
| 6481 | atgggaggcc tgaaccccca gggccaggcc ttgaacatca tgaacccagg |
| | acacaacccc |
| 6541 | aacatggcga gtatgaatcc acagtaccga gaaatgttac ggaggcagct |
| | gctgcagcag |
| 6601 | cagcagcaac agcagcagca acaacagcag caacagcagc agcagcaagg |
| | gagtgccggc |
| 6661 | atggctgggg gcatggcggg gcacggccag ttccagcagc ctcaaggacc |
| | cggaggctac |
| 6721 | ccaccggcca tgcagcagca gcagcgcatg cagcagcatc tccccctcca |
| | gggcagctcc |
| 6781 | atgggccaga tggcggctca gatgggacag cttggccaga tggggcagcc |
| | ggggctgggg |
| 6841 | gcagacagca cccccaacat ccagcaagcc ctgcagcagc ggattctgca |
| | gcaacagcag |
| 6901 | atgaagcagc agattgggtc cccaggccag ccgaacccca tgagccccca |
| | gcaacacatg |
| 6961 | ctctcaggac agccacaggc ctcgcatctc cctggccagc agatcgccac |
| | gtcccttagt |
| 7021 | aaccaggtgc ggtctccagc ccctgtccag tctccacggc cccagtccca |
| | gcctccacat |
| 7081 | tccagcccgt caccacggat acagccccag ccttcgccac accacgtctc |
| | accccagact |
| 7141 | ggttcccccc accccggact cgcagtcacc atggccagct ccatagatca |
| | gggacacttg |
| 7201 | gggaaccccg aacagagtgc aatgctcccc cagctgaaca cccccagcag |
| | gagtgcgctg |
| 7261 | tccagcgaac tgtccctggt cggggacacc acgggggaca cgctagagaa |
| | gtttgtggag |
| 7321 | ggcttgtag |

CREBBP Protein (Homo sapiens)

| SEQ ID NO: 46 | |
|---|---|
| 1 | maenlldgpp npkraklssp gfsandstdf gslfdlendl pdelipngge |
| | lgllnsgnlv |
| 61 | pdaaskhkql sellrggsgs sinpgignvs asspvqqglg gqaqgqpnsa |
| | nmaslsamgk |
| 121 | splsqgdssa pslpkqaast sgptpaasqa lnpqaqkqvg latsspatsq |
| | tgpgicmnan |
| 181 | fnqthpglln snsghslinq asqgqaqvmn gslgaagrgr gagmpyptpa |
| | mqgasssvla |
| 241 | etltqvspqm tghaglntaq aggmakmgit gntspfgqpf sqaggqpmga |
| | tgvnpqlask |
| 301 | qsmvnslptf ptdikntsvt nvpnmsqmqt svgivptqai atgptadpek |
| | rkliqqqlvl |
| 361 | llhahkcqrr eqangevrac slphcrtmkn vlnhmthcqa gkacqvahca |
| | ssrqiishwk |
| 421 | nctrhdcpvc lplknasdkr nqqtilgspa sgiqntigsv gtgqqnatsl |
| | snpnpidpss |
| 481 | mqrayaalgl pymnqpqtql qpqvpgqqpa qpqthqqmrt lnplgnnpmn |
| | ipaggittdq |
| 541 | qppnlisesa lptslgatnp lmndgsnsgn igtlstipta appsstgvrk |
| | gwhehvtqdl |
| 601 | rshlvhklvq aifptpdpaa lkdrrmenlv ayakkvegdm yesansrdey |
| | yhllaekiyk |
| 661 | iqkeleekrr srlhkqgilg nqpalpapga qppvipqaqp vrppngplsl |
| | pvnrmqvsqg |
| 721 | mnsfnpmslg nvqlpqapmg praaspmnhs vqmnsmgsvp gmaispsrmp |
| | qppnmmgaht |
| 781 | nnmmaqapaq sqflpqnqfp sssgamsvgm gqppaqtgvs qgqvpgaalp |
| | nplnmlgpqa |
| 841 | sqlpcppvtq splhptpppa staagmpslq httppgmtpp qpaaptqpst |
| | pvsssgqtpt |
| 901 | ptpgsvpsat qtqstptvqa aaqaqvtpqp qtpvqppsva tpqssqqqpt |
| | pvhaqppgtp |
| 961 | lsqaaasidn rvptpssvas aetnsqqpgp dvpvlemkte tqaedtepdp |
| | geskgeprse |
| 1021 | mmeedlqgas qvkeetdiae qksepmevde kkpevkvevk eeeesssngt |
| | asqstspsqp |
| 1081 | rkkifkpeel rqalmptlea lyrqdpeslp frqpvdpqll gipdyfdivk |
| | npmdlstikr |
| 1141 | kldtgqyqep wqyvddvwlm fnnawlynrk tsrvykfcsk laevfeqeid |
| | pvmqslgycc |
| 1201 | grkyefspqt lccygkqlct iprdaayysy qnryhfcekc fteiqgenvt |
| | lgddpsqpqt |
| 1261 | tiskdqfekk kndtldpepf vdckecgrkm hqicvlhydi iwpsgfvcdn |
| | clkktgrprk |
| 1321 | enkfsakrlq ttrlgnhled rvnkflrrqn hpeagevfvr vvassdktve |
| | vkpgmksrfv |
| 1381 | dsgemsesfp yrtkalfafe eidgvdvcff gmhvqeygsd cpppntrrvy |
| | isyldsihff |
| 1441 | rprclrtavy heiligyley vkklgyvtgh iwacppsegd dyifhchppd |
| | qkipkpkrlq |
| 1501 | ewykkmldka faeriihdyk difkqatedr ltsakelpyf egdfwpnvle |
| | esikeleqee |
| 1561 | eerkkeesta asettegsqg dsknakkknn kktnknkssi srankkkpsm |
| | pnvsndlsqk |
| 1621 | lyatmekhke vffvihlhag pvintlppiv dpdpllscdl mdgrdafltl |
| | ardkhwefss |
| 1681 | lrrskwstlc mlvelhtqgq drfvytcnec khhvetrwhc tvcedydlci |
| | ncyntkshah |
| 1741 | kmvkwglgld degssqgepq skspqesrrl siqrciqslv hacqcrnanc |
| | slpscqkmkr |
| 1801 | vvqhtkgckr ktnggcpvck qlialccyha khcqenkcpv pfclnikhkl |
| | rqqqiqhrlq |
| 1861 | qaqlmrrrma tmntrnvpqq slpsptsapp gtptqqpstp qtpqppaqpq |
| | pspvsmspag |
| 1921 | fpsvartqpp ttvstgkpts qvpappppaq pppaaveaar qiereaqqqq |
| | hlyrvninns |
| 1981 | mppgrtgmgt pgsqmapvsl nvprpnqvsg pvmpsmppgq wqqaplpqqq |
| | pmpglprpvi |
| 2041 | smqaqaavag prmpsvqppr sispsalqdl lrtlkspssp qqqqqvlnil |
| | ksnpqlmaaf |
| 2101 | ikqrtakyva nqpgmqpqpg lqsqpgmqpq pgmhqqpslq nlnamqagvp |
| | rpgvppqqqa |
| 2161 | mgglnpqgqa lnimnpghnp nmasmnpqyr emlrrqllqg qqqqqqqqqq |
| | qqqqqqgsag |
| 2221 | maggmaghgq fqqpqgpggy ppamqqqqrm qqhlplqgss mgqmaaqmgq |
| | lgqmgqpglg |
| 2281 | adstpniqqa lggrilqqqg mkgqigspgq pnpmspqqhm lsgqpgashl |
| | pgqqiatsls |
| 2341 | nqvrspapvq sprpqsqpph sspspriqpq psphhvspqt gsphpglavt |
| | massidqghl |
| 2401 | gnpeqsamlp qlntpsrsal sselslvgdt tgdtlekfve gl |

C2ta cDNA (Homo sapiens)

| SEQ ID NO: 47 | |
|---|---|
| 1 | atgcgttgcc tggctccacg ccctgctggg tcctacctgt cagagcccca |
| | aggcagctca |
| 61 | cagtgtgcca ccatggagtt ggggccccta gaaggtggct acctggagct |
| | tcttaacagc |
| 121 | gatgctgacc ccctgtgcct ctaccacttc tatgaccaga tggacctggc |
| | tggagaagaa |
| 181 | gagattgagc tctactcaga acccgacaca gacaccatca actgcgacca |
| | gttcagcagg |
| 241 | ctgttgtgtg acatggaagg tgatgaagag accagggagg cttatgccaa |
| | tatcgcggaa |
| 301 | ctggaccagt atgtcttcca ggactcccag ctggagggcc tgagcaagga |
| | cattttcaag |
| 361 | cacataggac cagatgaagt gatcggtgag agtatggaga tgccagcaga |
| | agttgggcag |
| 421 | aaaagtcaga aaagaccctt cccagaggag cttccggcag acctgaagca |
| | ctggaagcca |
| 481 | gctgagcccc ccactgtggt gactggcagt ctcctagtgg gaccagtgag |
| | cgactgctcc |
| 541 | accctgccct gcctgccact gcctgcgctg ttcaaccagg agccagcctc |
| | cggccagatg |
| 601 | cgcctggaga aaaccgacca gattcccatg cctttctcca gttcctcgtt |
| | gagctgcctg |
| 661 | aatctccctg agggacccat ccagtttgtc cccaccatct ccactctgcc |
| | ccatgggctc |
| 721 | tggcaaatct ctgaggctgg aacaggggtc tccagtatat tcatctacca |
| | tggtgaggtg |
| 781 | ccccaggcca gccaagtacc ccctcccagt ggattcactg tccacggcct |
| | cccaacatct |
| 841 | ccagaccggc caggctccac cagccccttc gctccatcag ccactgacct |
| | gcccagcatg |
| 901 | cctgaacctg ccctgacctc ccgagcaaac atgacagagc acaagacgtc |
| | ccccacccaa |
| 961 | tgcccggcag ctggagaggt ctccaacaag cttccaaaat ggcctgagcc |
| | ggtggagcag |
| 1021 | ttctaccgct cactgcagga cacgtatggt gccgagcccg caggcccgga |
| | tggcatccta |
| 1081 | gtggaggtgg atctggtgca ggccaggctg gagaggagca gcagcaagag |
| | cctggagcgg |
| 1141 | gaactggcca ccccggactg ggcagaacgg cagctggccc aaggaggcct |
| | ggctgaggtg |
| 1201 | ctgttggctg ccaaggagca ccggcggccg cgtgagacac gagtgattgc |
| | tgtgctgggc |
| 1261 | aaagctggtc agggcaagag ctattgggct ggggcagtga gccgggcctg |
| | ggcttgtggc |
| 1321 | cggcttcccc agtacgactt tgtcttctct gtcccctgcc attgcttgaa |
| | ccgtccgggg |
| 1381 | gatgcctatg gcctgcagga tctgctcttc tccctgggcc cacagccact |
| | cgtggcggcc |
| 1441 | gatgaggttt tcagccacat cttgaagaga cctgaccgcg ttctgctcat |
| | cctagacggc |
| 1501 | ttcgaggagc tggaagcgca agatggcttc ctgcacagca cgtgcggacc |
| | ggcaccggcg |
| 1561 | gagccctgct ccctccgggg gctgctggcc ggccttttcc agaagaagct |
| | gctccgaggt |
| 1621 | tgcaccctcc tcctcacagc ccggccccgg ggccgcctgg tccagagcct |
| | gagcaaggcc |
| 1681 | gacgccctat ttgagctgtc cggcttctcc atggagcagg cccaggcata |
| | cgtgatgcgc |
| 1741 | tactttgaga gctcagggat gacagagcac caagacagag ccctgacgct |
| | cctccgggac |
| 1801 | cggccacttc ttctcagtca cagccacagc cctactttgt gccgggcagt |
| | gtgccagctc |
| 1861 | tcagaggccc tgctggagct tggggaggac gccaagctgc cctccacgct |
| | cacgggactc |
| 1921 | tatgtcggcc tgctgggccg tgcagccctc gacagccccc ccggggccct |
| | ggcagagctg |
| 1981 | gccaagctgg cctgggagct gggccgcaga catcaaagta ccctacagga |
| | ggaccagttc |
| 2041 | ccatccgcag acgtgaggac ctgggcgatg gccaaaggct tagtccaaca |
| | cccaccgcgg |
| 2101 | gccgcagagt ccgagctggc cttccccagc ttcctcctgc aatgcttcct |
| | gggggccctg |
| 2161 | tggctggctc tgagtggcga aatcaaggac aaggagctcc cgcagtacct |
| | agcattgacc |
| 2221 | ccaaggaaga agaggcccta tgacaactgg ctggagggcg tgccacgctt |
| | tctggctggg |
| 2281 | ctgatcttcc agcctcccgc ccgctgcctg ggagccctac tcgggccatc |
| | ggcggctgcc |
| 2341 | tcggtggaca ggaagcagaa ggtgcttgcg aggtacctga agcggctgca |
| | gccggggaca |
| 2401 | ctgcgggcgc ggcagctgct ggagctgctg cactgcgccc acgaggccga |
| | ggaggctgga |
| 2461 | atttggcagc acgtggtaca ggagctcccc ggccgcctct cttttctggg |
| | cacccgcctc |
| 2521 | acgcctcctg atgcacatgt actgggcaag gccttggagg cggcgggcca |
| | agacttctcc |
| 2581 | ctggacctcc gcagcactgg catttgcccc tctggattgg ggagcctcgt |
| | gggactcagc |
| 2641 | tgtgtcaccc gtttcagggc tgccttgagc gacacggtgg cgctgtggga |
| | gtccctgcag |
| 2701 | cagcatgggg agaccaagct acttcaggca gcagaggaga agttcaccat |
| | cgagcctttc |
| 2761 | aaagccaagt ccctgaagga tgtggaagac ctgggaaagc ttgtgcagac |
| | tcagaggacg |
| 2821 | agaagttcct cggaagacac agctggggag ctccctgctg ttcgggacct |
| | aaagaaactg |
| 2881 | gagtttgcgc tgggccctgt ctcaggcccc caggctttcc ccaaactggt |
| | gcggatcctc |
| 2941 | acggcctttt cctccctgca gcatctggac ctggatgcgc tgagtgagaa |
| | caagatcggg |
| 3001 | gacgagggtg tctcgcagct ctcagccacc ttcccccagc tgaagtcctt |
| | ggaaaccctc |
| 3061 | aatctgtccc agaacaacat cactgacctg ggtgcctaca aactcgccga |
| | ggccctgcct |
| 3121 | tcgctcgctg catccctgct caggctaagc ttgtacaata actgcatctg |
| | cgacgtggga |
| 3181 | gccgagagct tggctcgtgt gcttccggac atggtgtccc tccgggtgat |
| | ggacgtccag |
| 3241 | tacaacaagt tcacggctgc cggggcccag cagctcgctg ccagccttcg |
| | gaggtgtcct |
| 3301 | catgtggaga cgctggcgat gtggacgccc accatcccat tcagtgtcca |
| | ggaacacctg |
| 3361 | caacaacagg attcacggat cagcctgaga t |

C2TA Protein (Homo sapiens)

| SEQ ID NO: 48 | |
|---|---|
| 1 | mrclaprpag sylsepqgss qcatmelgpl eggylellns dadplclyhf |
| | ydqmdlagee |
| 61 | eielysepdt dtincdqfsr llcdmegdee treayaniae ldqyvfqdsq |
| | leglskdifk |
| 121 | higpdevige smempaevgq ksqkrpfpee lpadlkhwkp aepptvvtgs |
| | llvgpvsdcs |
| 181 | tlpclplpal fnqepasgqm rlektdqipm pfsssslscl nlpegpiqfv |
| | ptistlphgl |
| 241 | wqiseagtgv ssifiyhgev pqasqvppps gftvhglpts pdrpgstspf |
| | apsatdlpsm |
| 301 | pepaltsran mtehktsptq cpaagevsnk lpkwpepveq fyrslqdtyg |
| | aepagpdgil |
| 361 | vevdlvqarl ersssksler elatpdwaer qlaqgglaev llaakehrrp |
| | retrviavlg |
| 421 | kagqgksywa gavsrawacg rlpqydfvfs vpchclnrpg dayglqdllf |
| | slgpqplvaa |
| 481 | devfshilkr pdrvllildg feeleaqdgf lhstcgpapa epcslrglla |
| | glfqkkllrg |
| 541 | ctllltarpr grlvqslska dalfelsgfs meqaqayvmr yfessgmteh |
| | qdraltllrd |
| 601 | rplllshshs ptlcravcql seallelged aklpstltgl yvgllgraal |
| | dsppgalael |
| 661 | aklawelgrr hqstlqedqf psadvrtwam akglvqhppr aaeselafps |
| | fllqcflgal |
| 721 | wlalsgeikd kelpqylalt prkkrpydnw legvprflag lifqpparcl |
| | gallgpsaaa |
| 781 | svdrkqkvla rylkrlqpgt lrarqllell hcaheaeeag iwqhvvqelp |
| | grlsflgtrl |
| 841 | tppdahvlgk aleaagqdfs ldlrstgicp sglgslvgls cvtrfraals |
| | dtvalweslq |
| 901 | qhgetkllqa aeekftiepf kakslkdved lgklvqtqrt rsssedtage |
| | lpavrdlkkl |
| 961 | efalgpvsgp qafpklvril tafsslqhld ldalsenkig degvsqlsat |
| | fpqlksletl |
| 1021 | nlsqnnitdl gayklaealp slaasllrls lynncicdvg aeslarvlpd |
| | mvslrvmdvq |
| 1081 | ynkftaagaq qlaaslrrcp hvetlamwtp tipfsvqehl qqqdsrislr |

Mxi1 cDNA (Homo sapiens)

SEQ ID NO: 49

| | |
|---|---|
| 1 | atggagcggg tgaagatgat caacgtgcag cgtctgctgg |
| | aggctgccga gtttttggag |
| 61 | cgccgggagc gagagtgtga acatggctac gcctcttcat |
| | tcccgtccat gccgagcccc |
| 121 | cgactgcagc attcaaagcc cccacggagg ttgagccggg |
| | cacagaaaca cagcagcggg |
| 181 | agcagcaaca ccagcactgc caacagatct acacacaatg |
| | agctggaaaa gaatcgacga |
| 241 | gctcatctgc gcctttgttt agaacgctta aaagttctga |
| | ttccactagg accagactgc |
| 301 | acccggcaca caacacttgg tttgctcaac aaagccaaag |
| | cacacatcaa gaaacttgaa |
| 361 | gaagctgaaa gaaaaagcca gcaccagctc gagaatttgg |
| | aacgagaaca gagattttta |
| 421 | aagtggcgac tggaacagct gcagggtcct caggagatgg |
| | aacgaatacg aatggacagc |
| 481 | attggatcaa ctatttcttc agatcgttct gattcagagc |
| | gagaggagat tgaagtggat |
| 541 | gttgaaagca cagagttctc ccatggagaa gtggacaata |
| | taagtaccac cagcatcagt |
| 601 | gacattgatg accacagcag cctgccgagt attgggagtg |
| | acgagggtta ctccagtgcc |
| 661 | agtgtcaaac tttcattcac ttcatag |

MXI1 Protein (Homo sapiens)

SEQ ID NO: 50

| | |
|---|---|
| 1 | mervkminvq rlleaaefle rrerecehgy assfpsmpsp |
| | rlqhskpprr lsraqkhssg |
| 61 | ssntstanrs thneleknrr ahlrlclerl kvliplgpdc |
| | trhttlglln kakahikkle |
| 121 | eaerksqhql enlereqrfl kwrleqlqgp qemerirmds |
| | igstissdrs dsereeievd |
| 181 | vestefshge vdnisttsis diddhsslps igsdegyssa |
| | svklsfts |

Hes3 cDNA (Homo sapiens)

SEQ ID NO: 51

| | |
|---|---|
| 1 | atggagaaaa agcgccgggc acgcatcaat gtgtcactgg |
| | agcagctcaa gtcgctgctg |
| 61 | gagaaacact actcgcacca gatccggaag cgcaaattgg |
| | agaaggccga catcctggag |
| 121 | ttgagcgtga agtacatgag aagccttcag aactccttgc |
| | aagggctctg gcctgtgccc |
| 181 | aggggagccg agcaaccgtc gggcttccgc agctgcctgc |
| | ccggcgtgag ccagctcctt |
| 241 | cggcgcggag atgaggtcgg cagcggcctg cgctgccccc |
| | tggtgcccga gagcgccgcc |
| 301 | ggcagcacca tggacagcgc cgggttgggc caggaggcgc |
| | ccgcgctgtt ccgcccttgc |
| 361 | acccctgccg tctgggctcc tgctccggcc gccggcggcc |
| | cgcggtcccc accacccctg |
| 421 | ctcctcctcc ccgaaagtct ccctggctcg tccgccagcg |
| | tccccccgcc gcagccagcg |
| 481 | tcgagtcgct gcgccgagag tcccgggctg ggcctgcgcg |
| | tgtggcggcc ctggggaagc |
| 541 | cccggggatg acctgaactg a |

HES3 Protein (Homo sapiens)

SEQ ID NO: 52

| | |
|---|---|
| 1 | mekkrrarin vsleqlksll ekhyshqirk rklekadile |
| | lsvkymrslq nslqglwpvp |
| 61 | rgaeqpsgfr sclpgvsqll rrgdevgsgl rcplvpesaa |
| | gstmdsaglg qeapalfrpc |
| 121 | tpavwapapa aggprspppl lllpeslpgs sasvpppqpa |
| | ssrcaespgl glrvwrpwgs |
| 181 | pgddln |

Rpl22 cDNA (Homo sapiens)

SEQ ID NO: 53

| | |
|---|---|
| 1 | atggctcctg tgaaaaagct tgtggtgaag gggggcaaaa |
| | aaaagaagca agttctgaag |
| 61 | ttcactcttg attgcaccca ccctgtagaa gatggaatca |
| | tggatgctgc caattttgag |
| 121 | cagtttttgc aagaaaggat caaagtgaac ggaaaagctg |
| | ggaaccttgg tggaggggtg |
| 181 | gtgaccatcg aaaggagcaa gagcaagatc accgtgacat |
| | ccgaggtgcc tttctccaaa |
| 241 | aggtatttga aatatctcac caaaaaatat ttgaagaaga |
| | ataatctacg tgactggttg |
| 301 | cgcgtagttg ctaacagcaa agagagttac gaattacgtt |
| | acttccagat taaccaggac |
| 361 | gaagaagagg aggaagacga ggattaa |

RPL22 Protein (Homo sapiens)

SEQ ID NO: 54

| | |
|---|---|
| 1 | mapvkklvvk ggkkkkqvlk ftldcthpve dgimdaanfe |
| | qflqerikvn gkagnlgggv |
| 61 | vtierskski tvtsevpfsk rylkyltkky lkknnlrdwl |
| | rvvanskesy elryfqinqd |
| 121 | eeeeeded |

Chd5 cDNA (Homo sapiens)

| SEQ ID NO: 55 | |
|---|---|
| 1 | atgcggggcc cagtgggcac cgaggaggag ctgccgcggc tgttcgccga |
| | ggagatggag |
| 61 | aatgaggacg agatgtcaga agaagaagat ggtggtcttg aagccttcga |
| | tgactttttc |
| 121 | cctgtggagc ccgtgagcct tcctaagaag aagaaaccca agaagctcaa |
| | ggaaaacaag |
| 181 | tgtaaaggga agcggaagaa gaaagagggg agcaatgatg agctatcaga |
| | gaatgaagag |
| 241 | gatctggaag agaagtcgga gagtgaaggc agtgactact ccccgaataa |
| | aaagaagaag |
| 301 | aagaaactca aggacaagaa ggagaaaaaa gccaagcgaa aaaagaagga |
| | tgaggatgag |
| 361 | gatgataatg atgatggatg cttaaaggag cccaagtcct cggggcagct |
| | catggccgag |
| 421 | tggggcctgg acgacgtgga ctacctgttc tcggaggagg attaccacac |
| | gctgaccaac |
| 481 | tacaaggcct tcagccagtt cctcaggcca ctcattgcca agaagaaccc |
| | gaagatcccc |
| 541 | atgtccaaaa tgatgaccgt cctgggtgcc aagtggcggg agttcagcgc |
| | caacaacccc |
| 601 | ttcaagggca gctccgcggc agcagcggcg gcggcggtgg ctgcggctgt |
| | agagacggtc |
| 661 | accatctccc ctccgctagc cgtcagcccc ccgcaggtgc cccagcctgt |
| | gcctatccgc |
| 721 | aaggccaaga ccaaggaggg caaagggcct ggagtgagga agaagatcaa |
| | aggctccaaa |
| 781 | gatgggaaga aaaagggcaa agggaaaaag acggccgggc tcaagttccg |
| | cttcgggggg |
| 841 | atcagcaaca agaggaagaa aggctcctcg agtgaagaag atgagaggga |
| | ggagtcggac |
| 901 | ttcgacagcg ccagcatcca cagtgcctcc gtgcgctccg aatgctctgc |
| | agccctgggc |
| 961 | aagaagagca agaggaggcg caagaagaag aggattgatg atggtgacgg |
| | ctatgagaca |
| 1021 | gaccaccagg attactgtga ggtgtgccag cagggtgggg agatcatcct |
| | gtgcgacacc |
| 1081 | tgcccgaggg cctaccatct cgtatgcctg gacccagagc tggagaaggc |
| | tcccgagggc |
| 1141 | aagtggagct gcccccactg tgagaaggag gggatccagt gggagccgaa |
| | ggacgacgac |
| 1201 | gatgaagagg aggagggcgg ctgcgaggag gaggaggacg accacatgga |
| | gttctgccgc |
| 1261 | gtgtgcaagg acgggggcga gctgctctgc tgcgacgcct gcccctcctc |
| | ctaccacctg |
| 1321 | cattgcctca acccgccgct gcccgagatc ccaaacggtg aatggctctg |
| | cccgcgctgt |
| 1381 | acttgccccc cactgaaggg caaagtccag cggattctac actggaggtg |
| | gacggagccc |
| 1441 | cctgccccct tcatggtggg gctgccgggg cctgacgtgg agcccagcct |
| | ccctccacct |
| 1501 | aagcccctgg agggcatccc tgagagagag ttctttgtca agtgggcagg |
| | gctgtcctac |
| 1561 | tggcattgct cctgggtgaa ggagctacag ctggagctgt accacacggt |
| | gatgtatcgc |
| 1621 | aactaccaaa gaaagaacga catggatgag ccgcccccct ttgactacgg |
| | ctctggggat |
| 1681 | gaagacggca agagcgagaa gaggaagaac aaggaccccc tctatgccaa |
| | gatggaggag |
| 1741 | cgcttctacc gctatggcat caagccagag tggatgatga ttcaccgaat |
| | cctgaaccat |
| 1801 | agctttgaca agaaggggga tgtgcactac ctgatcaagt ggaaagacct |
| | gccctacgac |
| 1861 | cagtgcacct gggagatcga tgacatcgac atcccctact acgacaacct |
| | caagcaggcc |
| 1921 | tactggggcc acagggagct gatgctggga gaagacacca ggctgcccaa |
| | gaggctgctc |
| 1981 | aagaagggca agaagctgag ggacgacaag caggagaagc cgccggacac |
| | gcccattgtg |
| 2041 | gaccccacgg tcaagttcga caagcagcca tggtacatcg actccacagg |
| | cggcacactg |
| 2101 | cacccgtacc agctggaggg cctcaactgg ctgcgcttct cttgggccca |
| | gggcactgac |
| 2161 | accatcctgg ccgatgagat gggtctgggc aagacggtgc agaccatcgt |
| | gttcctttac |
| 2221 | tccctctaca aggagggcca ctccaaaggg ccctacctgg ttagcgcgcc |
| | cctctccacc |
| 2281 | atcatcaact gggaacgcga gtttgagatg tgggcgcccg acttctacgt |
| | ggtcacctac |
| 2341 | acgggggaca aggagagccg ctcggtgatt cgggagaacg agttttcctt |
| | tgaggacaac |
| 2401 | gccattcgga gtgggaagaa ggtattccgt atgaagaaag aagtgcagat |
| | caaattccac |
| 2461 | gtgctgctca cctcctatga gctcatcacc attgaccagg ccatcctggg |
| | ctccatcgag |
| 2521 | tgggcctgcc tggtggtaga tgaggcccac cgcctcaaga acaaccagtc |
| | caagtttttt |
| 2581 | agggtcttaa acagctacaa gattgattac aagctgctgc tgacagggac |
| | cccccttcag |
| 2641 | aacaacctgg aggagctgtt ccatctcctc aacttcctga ctccagagag |
| | gttcaacaac |
| 2701 | ctggagggct tcctggagga gtttgctgac atctccaagg aagaccagat |
| | caagaagctg |
| 2761 | catgacctgc tggggccgca catgctcagg cggctcaagg ctgacgtgtt |
| | caagaacatg |
| 2821 | ccggccaaga ccgagctcat tgtccgggtg gagctgagcc agatgcagaa |
| | gaagtactac |
| 2881 | aagttcatcc tcacacggaa ctttgaggca ctgaactcca aggggggcgg |
| | gaaccaagta |
| 2941 | tcgctgctca acatcatgat ggacctgaaa aagtgctgca accaccccta |
| | cctcttccct |
| 3001 | gtggctgccg tggaggcccc tgtcttgccc aatggctcct acgatggaag |
| | ctccctggtc |
| 3061 | aagtcttcag ggaagctcat gctgctacag aagatgctga agaaactgcg |
| | ggatgagggg |
| 3121 | caccgtgtgc tcatcttctc ccagatgacc aagatgctgg acctcctgga |
| | ggacttcctg |
| 3181 | gagtacgaag gctacaagta tgagcggatt gatggtggca tcaccggggg |
| | cctccggcag |
| 3241 | gaggcaatcg acagattcaa tgcccccggg gcccagcagt tctgcttcct |
| | cctctcaacc |
| 3301 | cgggcaggtg gtctgggcat caacctggcc acggcggaca ctgtcatcat |
| | ctacgactcg |
| 3361 | gactggaacc cgcacaatga catccaggcc ttcagccgcg cccaccgcat |
| | cggccagaac |
| 3421 | aagaaggtga tgatctaccg cttcgtgact cgggcctcgg tggaggagcg |
| | catcacgcag |
| 3481 | gtggccaagc gcaagatgat gctcacccac ctggtggtgc ggcccggcct |
| | cggctccaag |
| 3541 | tcggggtcca tgaccaagca ggagctggac gacatcctca agttcggcac |
| | ggaggaactc |
| 3601 | ttcaaggacg acgtggaggg catgatgtct cagggccaga ggccggtcac |
| | acccatccct |
| 3661 | gatgtccagt cctccaaagg ggggaacttg gccgccagtg caaagaagaa |
| | gcacggtagc |
| 3721 | accccgccag gtgacaacaa ggacgtggag gacagcagtg tgatccacta |
| | tgacgatgcg |
| 3781 | gccatctcca agctgctgga ccggaaccag gacgctacag atgacacgga |
| | gctacagaac |
| 3841 | atgaacgagt acctgagctc cttcaaggtg gcgcagtacg tggtgcgcga |
| | ggaggacggc |
| 3901 | gtggaggagg tggagcggga aatcatcaag caggaggaga acgtggaccc |
| | cgactactgg |
| 3961 | gagaagctgc tgcggcacca ctatgagcag cagcaggagg acctggcccg |
| | caacctgggc |
| 4021 | aagggcaagc gcatccgcaa gcaggtcaac tacaacgatg cctcccagga |
| | ggaccaggag |
| 4081 | tggcaggatg agctctctga taaccagtca gaatattcca ttggctctga |
| | ggatgaggat |
| 4141 | gaggactttg aagagaggcc ggaagggcag agtggacgac gacaatcccg |
| | gaggcagctg |
| 4201 | aagagtgaca gggacaagcc cctgcccccg cttctcgccc gagttggtgg |
| | caacatcgag |
| 4261 | gtgctgggct tcaatgcccg acagcggaag gcctttctga acgccatcat |
| | gcgctggggc |
| 4321 | atgcccccgc aggacgcctt caactcccac tggctggtgc gggaccttcg |
| | agggaagagc |
| 4381 | gagaaggagt ttagagccta tgtgtccctc ttcatgcggc acctgtgtga |
| | gccgggggcg |
| 4441 | gatggtgcag agaccttcgc agacggcgtg ccccgggagg gcctctccag |
| | gcagcacgtg |
| 4501 | ctgacccgca tcggggtcat gtcactagtt aggaagaagg ttcaggagtt |
| | tgagcatgtc |
| 4561 | aacgggaagt acagcacccc agacttgatc cctgaggggc ccgaggggaa |
| | gaagtcgggc |
| 4621 | gaggtgatct cctcggaccc caacacacca gtgcccgcca gccctgccca |
| | cctcctgcca |
| 4681 | gccccgctgg gcctgccaga caaaatggaa gcccagctgg gctacatgga |
| | tgagaaagac |
| 4741 | cccggggcac agaagccaag gcagcccctg gaagtccagg cccttccagc |
| | cgccttggat |
| 4801 | agagtggaga gtgaggacaa gcacgagagc ccagccagca aggagagagc |
| | ccgagaggag |
| 4861 | cggccagagg agacggagaa ggccccgccc tccccggagc agctgccgag |
| | agaggaggtg |
| 4921 | cttcctgaga aggagaagat cctggacaag ctggagctga gcttgatcca |
| | cagcagaggg |
| 4981 | gacagttccg aactcaggcc agatgacacc aaggctgagg agaaggagcc |
| | cattgaaaca |
| 5041 | cagcaaaatg gtgacaaaga ggaagatgac gaggggaaga aggaggacaa |
| | gaaggggaaa |
| 5101 | ttcaagttca tgttcaacat cgcggacggg ggcttcacgg agttgcacac |
| | gctgtggcag |
| 5161 | aacgaggagc gggctgctgt atcctctggg aaaatctacg acatctggca |
| | ccggcgccat |
| 5221 | gactactggc tgctggcggg catcgtgacg cacggctacg cccgctggca |
| | ggacatccag |
| 5281 | aatgacccac ggtacatgat cctcaacgag cccttcaagt ctgaggtcca |
| | caagggcaac |
| 5341 | tacctggaga tgaagaacaa gttcctggcc cgcaggttta agctgctgga |
| | gcaggcgttg |
| 5401 | gtcattgagg agcagctccg gagggccgcg tacctgaaca tgacgcagga |
| | ccccaaccac |
| 5461 | cccgccatgg ccctcaacgc ccgcctggct gaagtggagt gcctcgccga |
| | gagccaccag |
| 5521 | cacctgtcca aggagtccct tgctgggaac aagcctgcca atgccgtcct |
| | gcacaaggtc |
| 5581 | ctgaaccagc tggaggagct gctgagcgac atgaaggccg acgtgacccg |
| | gctgccatcc |
| 5641 | atgctgtccc gcatcccccc ggtggccgcc cggctgcaga tgtcggagcg |
| | cagcatcctg |
| 5701 | agccgcctga ccaaccgcgc cggggacccc accatccagc agggcgcttt |
| | cggctcctcc |
| 5761 | cagatgtaca gcaacaactt tgggcccaac ttccggggcc ctggaccggg |
| | agggattgtc |
| 5821 | aactacaacc agatgcccct ggggccctat gtgaccgata tctag |

CHD5 Protein (Homo sapiens)

| SEQ ID NO: 56 | |
|---|---|
| 1 | mrgpvgteee lprlfaeeme nedemseeed ggleafddff pvepvslpkk |
| | kkpkklkenk |
| 61 | ckgkrkkkeg sndelsenee dleekseseg sdyspnkkkk kklkdkkekk |
| | akrkkkdede |
| 121 | ddnddgclke pkssgqlmae wglddvdylf seedyhtltn ykafsqflrp |
| | liakknpkip |
| 181 | mskmmtvlga kwrefsannp fkgssaaaaa aavaaavetv tispplavsp |
| | pqvpqpvpir |
| 241 | kaktkegkgp gvrkkikgsk dgkkkgkgkk taglkfrfgg isnkrkkgss |
| | seedereesd |
| 301 | fdsasihsas vrsecsaalg kkskrrrkkk riddgdgyet dhqdycevcq |
| | qggeiilcdt |
| 361 | cprayhlvcl dpelekapeg kwscphceke giqwepkddd deeeeggcee |
| | eeddhmefcr |
| 421 | vckdggellc cdacpssyhl hclnpplpei pngewlcprc tcpplkgkvq |
| | rilhwrwtep |
| 481 | papfmvglpg pdvepslppp kplegipere ffvkwaglsy whcswvkelq |
| | lelyhtvmyr |
| 541 | nyqrkndmde pppfdygsgd edgksekrkn kdplyakmee rfyrygikpe |
| | wmmihrilnh |
| 601 | sfdkkgdvhy likwkdlpyd qctweiddid ipyydnlkqa ywghrelmlg |
| | edtrlpkrll |
| 661 | kkgkklrddk qekppdtpiv dptvkfdkqp wyidstggtl hpyqleglnw |
| | lrfswaqgtd |
| 721 | tilademglg ktvqtivfly slykeghskg pylvsaplst iinwerefem |
| | wapdfyvvty |
| 781 | tgdkesrsvi renefsfedn airsgkkvfr mkkevqikfh vlltsyelit |
| | idqailgsie |
| 841 | waclvvdeah rlknnqskff rvlnsykidy kllltgtplq nnleelfhll |
| | nfltperfnn |
| 901 | legfleefad iskedqikkl hdllgphmlr rlkadvfknm paktelivrv |
| | elsqmqkkyy |
| 961 | kfiltrnfea lnskgggnqv sllnimmdlk kccnhpylfp vaaveapvlp |
| | ngsydgsslv |
| 1021 | kssgklmllq kmlkklrdeg hrvlifsqmt kmldlledfl eyegykyeri |
| | dggitgglrq |
| 1081 | eaidrfnapg aqqfcfllst ragglginla tadtviiyds dwnphndiqa |
| | fsrahrigqn |
| 1141 | kkvmiyrfvt rasveeritq vakrkmmlth lvvrpglgsk sgsmtkqeld |
| | dilkfgteel |
| 1201 | fkddvegmms qgqrpvtpip dvqsskggnl aasakkkhgs tppgdnkdve |
| | dssvihydda |
| 1261 | aisklldrnq datddtelqn mneylssfkv aqyvvreedg veevereiik |
| | qeenvdpdyw |
| 1321 | ekllrhhyeq qqedlarnlg kgkrirkqvn yndasqedqe wqdelsdnqs |
| | eysigseded |
| 1381 | edfeerpegq sgrrqsrrql ksdrdkplpp llarvggnie vlgfnarqrk |
| | aflnaimrwg |
| 1441 | mppqdafnsh wlvrdlrgks ekefrayvsl fmrhlcepga dgaetfadgv |
| | preglsrqhv |
| 1501 | ltrigvmslv rkkvqefehv ngkystpdli pegpegkksg evissdpntp |
| | vpaspahllp |
| 1561 | aplglpdkme aqlgymdekd pgaqkprqpl evqalpaald rvesedkhes |
| | paskeraree |
| 1621 | rpeetekapp speqlpreev lpekekildk lelslihsrg dsselrpddt |
| | kaeekepiet |
| 1681 | qqngdkeedd egkkedkkgk fkfmfniadg gftelhtlwq neeraavssg |
| | kiydiwhrrh |
| 1741 | dywllagivt hgyarwqdiq ndprymilne pfksevhkgn ylemknkfla |
| | rrfklleqal |
| 1801 | vieeqlrraa ylnmtqdpnh pamalnarla eveclaeshq hlskeslagn |
| | kpanavlhkv |
| 1861 | lnqleellsd mkadvtrlps mlsrippvaa rlqmsersil srltnragdp |
| | tiqqgafgss |
| 1921 | qmysnnfgpn frgpgpggiv nynqmplgpy vtdi |

Ikaros cDNA (Homo sapiens)

| SEQ ID NO: 57 | |
|---|---|
| 1 | atggatgctg atgagggtca agacatgtcc caagtttcag ggaaggaaag |
| | cccccctgta |
| 61 | agcgatactc cagatgaggg cgatgagccc atgccgatcc ccgaggacct |
| | ctccaccacc |
| 121 | tcgggaggac agcaaagctc caagagtgac agagtcgtgg ccagtaatgt |
| | taaagtagag |
| 181 | actcagagtg atgaagagaa tgggcgtgcc tgtgaaatga atggggaaga |
| | atgtgcggag |
| 241 | gatttacgaa tgcttgatgc ctcgggagag aaaatgaatg gctcccacag |
| | ggaccaaggc |
| 301 | agctcggctt tgtcgggagt tggaggcatt cgacttccta acggaaaact |
| | aaagtgtgat |
| 361 | atctgtggga tcatttgcat cgggcccaat gtgctcatgg ttcacaaaag |
| | aagccacact |
| 421 | ggagaacggc ccttccagtg caatcagtgc ggggcctcat tcacccagaa |
| | gggcaacctg |
| 481 | ctccggcaca tcaagctgca ttccggggag aagcccttca aatgccacct |
| | ctgcaactac |
| 541 | gcctgccgcc ggagggacgc cctcactggc cacctgagga cgcactccgt |
| | tggtaaacct |
| 601 | cacaaatgtg gatattgtgg ccgaagctat aaacagcgaa gctctttaga |
| | ggaacataaa |
| 661 | gagcgctgcc acaactactt ggaaagcatg ggccttccgg gcacactgta |
| | cccagtcatt |
| 721 | aaagaagaaa ctaatcacag tgaaatggca gaagacctgt gcaagatagg |
| | atcagagaga |
| 781 | tctctcgtgc tggacagact agcaagtaac gtcgccaaac gtaagagctc |
| | tatgcctcag |
| 841 | aaatttcttg gggacaaggg cctgtccgac acgccctacg acagcagcgc |
| | cagctacgag |
| 901 | aaggagaacg aaatgatgaa gtcccacgtg atggaccaag ccatcaacaa |
| | cgccatcaac |
| 961 | tacctggggg ccgagtccct gcgcccgctg gtgcagacgc ccccgggcgg |
| | ttccgaggtg |
| 1021 | gtcccggtca tcagcccgat gtaccagctg cacaagccgc tcgcggaggg |
| | caccccgcgc |
| 1081 | tccaaccact cggcccagga cagcgccgtg gagaacctgc tgctgctctc |
| | caaggccaag |
| 1141 | ttggtgccct cggagcgcga ggcgtccccg agcaacagct gccaagactc |
| | cacggacacc |
| 1201 | gagagcaaca acgaggagca gcgcagcggt ctcatctacc tgaccaacca |
| | catcgccccg |
| 1261 | cacgcgcgca acgggctgtc gctcaaggag gagcaccgcg cctacgacct |
| | gctgcgcgcc |
| 1321 | gcctccgaga actcgcagga cgcgctccgc gtggtcagca ccagcgggga |
| | gcagatgaag |
| 1381 | gtgtacaagt gcgaacactg ccgggtgctc ttcctggatc acgtcatgta |
| | caccatccac |
| 1441 | atgggctgcc acggcttccg tgatcctttt gagtgcaaca tgtgcggcta |
| | ccacagccag |
| 1501 | gaccggtacg agttctcgtc gcacataacg cgaggggagc accgcttcca |
| | catgagctaa |

IKAROS Protein (Homo sapiens)

| SEQ ID NO: 58 | |
|---|---|
| 1 | mdadegqdms qvsgkesppv sdtpdegdep mpipedlstt sggqqssksd |
| | rvvasnvkve |
| 61 | tqsdeengra cemngeecae dlrmldasge kmngshrdqg ssalsgvggi |
| | rlpngklkcd |
| 121 | icgiicigpn vlmvhkrsht gerpfqcnqc gasftqkgnl lrhiklhsge |
| | kpfkchlcny |
| 181 | acrrrdaltg hlrthsvgkp hkcgycgrsy kqrssleehk erchnylesm |
| | glpgtlypvi |
| 241 | keetnhsema edlckigser slvldrlasn vakrkssmpq kflgdkglsd |
| | tpydssasye |
| 301 | kenemmkshv mdqainnain ylgaeslrpl vqtppggsev vpvispmyql |
| | hkplaegtpr |
| 361 | snhsaqdsav enllllskak lvpsereasp snscqdstdt esnneeqrsg |
| | liyltnhiap |
| 421 | harnglslke ehraydllra asensqdalr vvstsgeqmk vykcehcrvl |
| | fldhvmytih |
| 481 | mgchgfrdpf ecnmcgyhsq dryefsshit rgehrfhms |

Ptprn2 cDNA (Homo sapiens)

| SEQ ID NO: 59 | |
|---|---|
| 1 | atggggccgc cgctcccgct gctgctgctg ctactgctgc tgctgccgcc |
| | acgcgtcctg |
| 61 | cctgccgccc cttcgtccgt cccccgcggc cggcagctcc cggggcgtct |
| | gggctgcctg |
| 121 | ctcgaggagg gcctctgcgg agcgtccgag gcctgtgtga acgatggagt |
| | gtttggaagg |
| 181 | tgccagaagg ttccggcaat ggacttttac cgctacgagg tgtcgcccgt |
| | ggccctgcag |
| 241 | cgcctgcgcg tggcgttgca gaagctttcc ggcacaggtt tcacgtggca |
| | ggatgactat |
| 301 | actcagtatg tgatggacca ggaacttgca gacctcccga aaacctacct |
| | gaggcgtcct |
| 361 | gaagcatcca gcccagccag gccctcaaaa cacagcgttg gcagcgagag |
| | gaggtacagt |
| 421 | cgggagggcg gtgctgccct ggccaacgcc ctccgacgcc acctgccctt |
| | cctggaggcc |
| 481 | ctgtcccagg ccccagcctc agacgtgctc gccaggaccc atacggcgca |
| | ggacagaccc |
| 541 | cccgctgagg gtgatgaccg cttctccgag agcatcctga cctatgtggc |
| | ccacacgtct |
| 601 | gcgctgacct accctcccgg gccccggacc cagctccgcg aggacctcct |
| | gccgcggacc |
| 661 | ctcggccagc tccagccaga tgagctcagc cctaaggtgg acagtggtgt |
| | ggacagacac |
| 721 | catctgatgg cggccctcag tgcctatgct gcccagaggc ccccagctcc |
| | ccccggggag |
| 781 | ggcagcctgg agccacagta ccttctgcgt gcaccctcaa gaatgcccag |
| | gcctttgctg |
| 841 | gcaccagccg ccccccagaa gtggccttca cctctgggag attccgaaga |
| | cccctccagc |
| 901 | acaggcgatg gagcacggat tcataccctc ctgaaggacc tgcagaggca |
| | gccggctgag |
| 961 | gtgaggggcc tgagtggcct ggagctggac ggcatggctg agctgatggc |
| | tggcctgatg |
| 1021 | caaggcgtgg accatggagt agctcgaggc agccctggga gagcggccct |
| | gggagagtct |
| 1081 | ggagaacagg cggatggccc caaggccacc ctccgtggag acagctttcc |
| | agatgacgga |
| 1141 | gtgcaggacg acgatgatag actttaccaa gaggtccatc gtctgagtgc |
| | cacactcggg |
| 1201 | ggcctcctgc aggaccacgg gtctcgactc ttacctggag ccctcccctt |
| | tgcaaggccc |
| 1261 | ctcgacatgg agaggaagaa gtccgagcac cctgagtctt ccctgtcttc |
| | agaagaggag |
| 1321 | actgccggag tggagaacgt caagagccag acgtattcca aagatctgct |
| | ggggcagcag |
| 1381 | ccgcattcgg agcccggggc cgctgcgttt ggggagctcc aaaaccagat |
| | gcctgggccc |
| 1441 | tcgaaggagg agcagagcct tccagcgggt gctcaggagg ccctcagcga |
| | cggcctgcaa |
| 1501 | ttggaggtcc agccttccga ggaagaggcg cggggctaca tcgtgacaga |
| | cagagacccc |
| 1561 | ctgcgccccg aggaaggaag gcggctggtg gaggacgtcg cccgcctcct |
| | gcaggtgccc |
| 1621 | agcagtgcgt tcgctgacgt ggaggttctc ggaccagcag tgaccttcaa |
| | agtgagcgcc |
| 1681 | aatgtccaaa acgtgaccac tgaggatgtg gagaaggcca cagttgacaa |
| | caaagacaaa |
| 1741 | ctggaggaaa cctctggact gaaaattctt caaaccggag tcgggtcgaa |
| | aagcaaactc |
| 1801 | aagttcctgc ctcctcaggc ggagcaagaa gactccacca agttcatcgc |
| | gctcaccctg |
| 1861 | gtctccctcg cctgcatcct gggcgtcctc ctggcctctg gcctcatcta |
| | ctgcctccgc |
| 1921 | catagctctc agcacaggct gaaggagaag ctctcgggac tagggggcga |
| | cccaggtgca |
| 1981 | gatgccactg ccgcctacca ggagctgtgc cgccagcgta tggccacgcg |
| | gccaccagac |
| 2041 | cgacctgagg gcccgcacac gtcacgcatc agcagcgtct catcccagtt |
| | cagcgacggg |
| 2101 | ccgatcccca gcccctccgc acgcagcagc gcctcatcct ggtccgagga |
| | gcctgtgcag |
| 2161 | tccaacatgg acatctccac cggccacatg atcctgtcct acatggagga |
| | ccacctgaag |
| 2221 | aacaagaacc ggctggagaa ggagtgggaa gcgctgtgcg cctaccaggc |
| | ggagcccaac |
| 2281 | agctcgttcg tggcccagag ggaggagaac gtgcccaaga accgctccct |
| | ggctgtgctg |
| 2341 | acctatgacc actcccgggt cctgctgaag gcggagaaca gccacagcca |
| | ctcagactac |
| 2401 | atcaacgcta gccccatcat ggatcacgac ccgaggaacc ccgcgtacat |
| | cgccacccag |
| 2461 | ggaccgctgc ccgccaccgt ggctgacttt tggcagatgg tgtgggagag |
| | cggctgcgtg |
| 2521 | gtgatcgtca tgctgacacc cctcgcggag aacggcgtcc ggcagtgcta |
| | ccactactgg |
| 2581 | ccggatgaag gctccaatct ctaccacatc tatgaggtga acctggtctc |
| | cgagcacatc |
| 2641 | tggtgtgagg acttcctggt gaggagcttc tatctgaaga acctgcagac |
| | caacgagacg |
| 2701 | cgcaccgtga cgcagttcca cttcctgagt tggtatgacc gaggagtccc |
| | ttcctcctca |
| 2761 | aggtccctcc tggacttccg cagaaaagta aacaagtgct acaggggccg |
| | ttcttgtcca |
| 2821 | ataattgttc attgcagtga cggtgcaggc cggagcggca cctacgtcct |
| | gatcgacatg |
| 2881 | gttctcaaca agatggccaa aggtgctaaa gagattgata tcgcagcgac |
| | cctggagcac |
| 2941 | ttgagggacc agagacccgg catggtccag acgaaggagc agtttgagtt |
| | cgcgctgaca |
| 3001 | gccgtggctg aggaggtgaa cgccatcctc aaggcccttc cccagtga |

PTPRN2 Protein (Homo sapiens)

| SEQ ID NO: 60 | |
|---|---|
| 1 | mgpplpllll lllllpprvl paapssvprg rqlpgrlgcl leeglcgase |
| | acvndgvfgr |
| 61 | cqkvpamdfy ryevspvalq rlrvalqkls gtgftwqddy tqyvmdqela |
| | dlpktylrrp |
| 121 | eassparpsk hsvgserrys reggaalana lrrhlpflea lsqapasdvl |
| | arthtaqdrp |
| 181 | paegddrfse siltyvahts altyppgprt qlredllprt lgqlqpdels |
| | pkvdsgvdrh |
| 241 | hlmaalsaya aqrppappge gslepqyllr apsrmprpll apaapqkwps |
| | plgdsedpss |
| 301 | tgdgarihtl lkdlqrqpae vrglsgleld gmaelmaglm qgvdhgvarg |
| | spgraalges |
| 361 | geqadgpkat lrgdsfpddg vqddddrlyq evhrlsatlg gllqdhgsrl |
| | lpgalpfarp |
| 421 | ldmerkkseh pesslsseee tagvenvksq tyskdllgqq phsepgaaaf |
| | gelqnqmpgp |
| 481 | skeeqslpag aqealsdglq levqpseeea rgyivtdrdp lrpeegrrlv |
| | edvarllqvp |
| 541 | ssafadvevl gpavtfkvsa nvqnvttedv ekatvdnkdk leetsglkil |
| | qtgvgskskl |
| 601 | kflppqaeqe dstkfialtl vslacilgvl lasgliyclr hssqhrlkek |
| | lsglggdpga |
| 661 | dataayqelc rqrmatrppd rpegphtsri ssvssqfsdg pipspsarss |
| | asswseepvq |
| 721 | snmdistghm ilsymedhlk nknrlekewe alcayqaepn ssfvaqreen |
| | vpknrslavl |
| 781 | tydhsrvllk aenshshsdy inaspimdhd prnpayiatq gplpatvadf |
| | wqmvwesgcv |
| 841 | vivmltplae ngvrqcyhyw pdegsnlyhi yevnlvsehi wcedflvrsf |
| | ylknlqtnet |
| 901 | rtvtqfhfls wydrgvpsss rslldfrrkv nkcyrgrscp iivhcsdgag |
| | rsgtyvlidm |
| 961 | vlnkmakgak eidiaatleh lrdqrpgmvq tkeqfefalt avaeevnail |
| | kalpq |

Tcrb cDNA (Partial Sequence) (Homo sapiens)

| SEQ ID NO: 61 | |
|---|---|
| 1 | atgggctgaa gtctccactg tggtgtggtc cattgtctca ggctccatgg |
| | atactggaat |
| 61 | tacccagaca ccaaaatacc tggtcacagc aatggggagt aaaaggacaa |
| | tgaaacgtga |
| 121 | gcatctggga catgattcta tgtattggta cagacagaaa gctaagaaat |
| | ccctggagtt |
| 181 | catgttttac tacaactgta aggaattcat tgaaaacaag actgtgccaa |
| | atcacttcac |
| 241 | acctgaatgc cctgacagct ctcgcttata ccttcatgtg gtcgcactgc |
| | agcaagaaga |
| 301 | ctcagctgcg tatctctgca ccagcagcca aga |

TCRB Protein (Homo sapiens)

| SEQ ID NO: 62 | |
|---|---|
| 1 | mgtsllcwma lcllgadhad tgvsqnprhn itkrgqnvtf rcdpisehnr |
| | lywyrqtlgq |
| 61 | gpefltyfqn eaqleksrll sdrfsaerpk gsfstleiqr teqgdsamyl |
| | casslaglnq |
| 121 | pqhfgdgtrl sil |

Gnaq cDNA (Homo sapiens)

| SEQ ID NO: 63 | |
|---|---|
| 1 | atgactctgg agtccatcat ggcgtgctgc ctgagcgagg aggccaagga |
| | agcccggcgg |
| 61 | atcaacgacg agatcgagcg gcagctccgc agggacaagc gggacgcccg |
| | ccgggagctc |
| 121 | aagctgctgc tgctcgggac aggagagagt ggcaagagta cgtttatcaa |
| | gcagatgaga |
| 181 | atcatccatg ggtcaggata ctctgatgaa gataaaaggg gcttcaccaa |
| | gctggtgtat |
| 241 | cagaacatct tcacggccat gcaggccatg atcagagcca tggacacact |
| | caagatccca |
| 301 | tacaagtatg agcacaataa ggctcatgca caattagttc gagaagttga |
| | tgtggagaag |
| 361 | gtgtctgctt ttgagaatcc atatgtagat gcaataaaga gtttatggaa |
| | tgatcctgga |
| 421 | atccaggaat gctatgatag acgacgagaa tatcaattat ctgactctac |
| | caaatactat |
| 481 | cttaatgact tggaccgcgt agctgaccct gcctacctgc ctacgcaaca |
| | agatgtgctt |
| 541 | agagttcgag tccccaccac agggatcatc gaatacccct ttgacttaca |
| | aagtgtcatt |
| 601 | ttcagaatgg tcgatgtagg gggccaaagg tcagagagaa gaaaatggat |
| | acactgcttt |
| 661 | gaaaatgtca cctctatcat gtttctagta gcgcttagtg aatatgatca |
| | agttctcgtg |
| 721 | gagtcagaca atgagaaccg aatggaggaa agcaaggctc tctttagaac |
| | aattatcaca |
| 781 | tacccctggt tccagaactc ctcggttatt ctgttcttaa acaagaaaga |
| | tcttctagag |
| 841 | gagaaaatca tgtattccca tctagtcgac tacttcccag aatatgatgg |
| | accccagaga |
| 901 | gatgcccagg cagcccgaga attcattctg aagatgttcg tggacctgaa |
| | cccagacagt |
| 961 | gacaaaatta tctactccca cttcacgtgc gccacagaca ccgagaatat |
| | ccgctttgtc |
| 1021 | tttgctgccg tcaaggacac catcctccag ttgaacctga aggagtacaa |
| | tctggtctaa |

GNAQ Protein (Homo sapiens)

| SEQ ID NO: 64 | |
|---|---|
| 1 | mtlesimacc lseeakearr indeierqlr rdkrdarrel kllllgtges |
| | gkstfikqmr |
| 61 | iihgsgysde dkrgftklvy gniftamqam iramdtlkip ykyehnkaha |
| | qlvrevdvek |
| 121 | vsafenpyvd aikslwndpg iqecydrrre yqlsdstkyy lndldrvadp |
| | aylptqqdvl |
| 181 | rvrvpttgii eypfdlqsvi frmvdvggqr serrkwihcf envtsimflv |
| | alseydqvlv |
| 241 | esdnenrmee skalfrtiit ypwfqnssvi lflnkkdlle ekimyshlvd |
| | yfpeydgpqr |
| 301 | daqaarefil kmfvdlnpds dkiiyshftc atdtenirfv faavkdtilq |
| | lnlkeynlv |

Pten cDNA (Homo sapiens)

| SEQ ID NO: 65 | |
|---|---|
| 1 | atgacagcca tcatcaaaga gatcgttagc agaaacaaaa ggagatatca |
| | agaggatgga |
| 61 | ttcgacttag acttgaccta tatttatcca aacattattg ctatgggatt |
| | tcctgcagaa |
| 121 | agacttgaag gcgtatacag gaacaatatt gatgatgtag taaggttttt |
| | ggattcaaag |
| 181 | cataaaaacc attacaagat atacaatctt tgtgctgaaa gacattatga |
| | caccgccaaa |
| 241 | tttaattgca gagttgcaca atatcctttt gaagaccata acccaccaca |
| | gctagaactt |
| 301 | atcaaaccct tttgtgaaga tcttgaccaa tggctaagtg aagatgacaa |
| | tcatgttgca |
| 361 | gcaattcact gtaaagctgg aaagggacga actggtgtaa tgatatgtgc |
| | atatttatta |
| 421 | catcggggca aatttttaaa ggcacaagag gccctagatt tctatgggga |
| | agtaaggacc |
| 481 | agagacaaaa agggagtaac tattcccagt cagaggcgct atgtgtatta |
| | ttatagctac |
| 541 | ctgttaaaga atcatctgga ttatagacca gtggcactgt tgtttcacaa |
| | gatgatgttt |
| 601 | gaaactattc caatgttcag tggcggaact tgcaatcctc agtttgtggt |
| | ctgccagcta |
| 661 | aaggtgaaga tatattcctc caattcagga cccacacgac gggaagacaa |
| | gttcatgtac |
| 721 | tttgagttcc ctcagccgtt acctgtgtgt ggtgatatca aagtagagtt |
| | cttccacaaa |
| 781 | cagaacaaga tgctaaaaaa ggacaaaatg tttcactttt gggtaaatac |
| | attcttcata |
| 841 | ccaggaccag aggaaacctc agaaaaagta gaaaatggaa gtctatgtga |
| | tcaagaaatc |
| 901 | gatagcattt gcagtataga gcgtgcagat aatgacaagg aatatctagt |
| | acttacttta |
| 961 | acaaaaaatg atcttgacaa agcaaataaa gacaaagcca accgatactt |
| | ttctccaaat |
| 1021 | tttaaggtga agctgtactt cacaaaaaca gtagaggagc cgtcaaatcc |
| | agaggctagc |
| 1081 | agttcaactt ctgtaacacc agatgttagt gacaatgaac ctgatcatta |
| | tagatattct |
| 1141 | gacaccactg actctgatcc agagaatgaa ccttttgatg aagatcagca |
| | tacacaaatt |
| 1201 | acaaaagtct ga |

PTEN Protein (Homo sapiens)

| SEQ ID NO: 66 | |
|---|---|
| 1 | mtaiikeivs rnkrryqedg fdldltyiyp niiamgfpae rlegvyrnni |
| | ddvvrfldsk |
| 61 | hknhykiynl caerhydtak fncrvaqypf edhnppqlel ikpfcedldq |
| | wlseddnhva |
| 121 | aihckagkgr tgvmicayll hrgkflkaqe aldfygevrt rdkkgvtips |
| | qrryvyyysy |
| 181 | llknhldyrp vallfhkmmf etipmfsggt cnpqfvvcql kvkiyssnsg |
| | ptrredkfmy |
| 241 | fefpqplpvc gdikveffhk qnkmlkkdkm fhfwvntffi pgpeetsekv |
| | engslcdqei |
| 301 | dsicsierad ndkeylvltl tkndldkank dkanryfspn fkvklyftkt |
| | veepsnpeas |
| 361 | sstsvtpdvs dnepdhyrys dttdsdpene pfdedqhtqi tkv |

Fbxw7 cDNA (Homo sapiens)

| SEQ ID NO: 67 | |
|---|---|
| 1 | atgaatcagg aactgctctc tgtgggcagc aaaagacgac gaactggagg |
| | ctctctgaga |
| 61 | ggtaaccctt cctcaagcca ggtagatgaa gaacagatga atcgtgtggt |
| | agaggaggaa |
| 121 | cagcaacagc aactcagaca acaagaggag gagcacactg caaggaatgg |
| | tgaagttgtt |
| 181 | ggagtagaac ctagacctgg aggccaaaat gattcccagc aaggacagtt |
| | ggaagaaaac |
| 241 | aataatagat ttatttcggt agatgaggac tcctcaggaa accaagaaga |
| | acaagaggaa |
| 301 | gatgaagaac atgctggtga acaagatgag gaggatgagg aggaggagga |
| | gatggaccag |
| 361 | gagagtgacg attttgatca gtctgatgat agtagcagag aagatgaaca |
| | tacacatact |
| 421 | aacagtgtca cgaactccag tagtattgtg gacctgcccg ttcaccaact |
| | ctcctcccca |
| 481 | ttctatacaa aaacaacaaa aatgaaaaga aagttggacc atggttctga |
| | ggtccgctct |
| 541 | ttttctttgg gaaagaaacc atgcaaagtc tcagaatata caagtaccac |
| | tgggcttgta |
| 601 | ccatgttcag caacaccaac aacttttggg gacctcagag cagccaatgg |
| | ccaagggcaa |
| 661 | caacgacgcc gaattacatc tgtccagcca cctacaggcc tccaggaatg |
| | gctaaaaatg |
| 721 | tttcagagct ggagtggacc agagaaattg cttgctttag atgaactcat |
| | tgatagttgt |
| 781 | gaaccaacac aagtaaaaca tatgatgcaa gtgatagaac cccagtttca |
| | acgagacttc |
| 841 | atttcattgc tccctaaaga gttggcactc tatgtgcttt cattcctgga |
| | acccaaagac |
| 901 | ctgctacaag cagctcagac atgtcgctac tggagaattt tggctgaaga |
| | caaccttctc |
| 961 | tggagagaga aatgcaaaga agaggggatt gatgaaccat tgcacatcaa |
| | gagaagaaaa |
| 1021 | gtaataaaac caggtttcat acacagtcca tggaaaagtg catacatcag |
| | acagcacaga |
| 1081 | attgatacta actggaggcg aggagaactc aaatctccta aggtgctgaa |
| | aggacatgat |
| 1141 | gatcatgtga tcacatgctt acagttttgt ggtaaccgaa tagttagtgg |
| | ttctgatgac |
| 1201 | aacactttaa aagtttggtc agcagtcaca ggcaaatgtc tgagaacatt |
| | agtgggacat |
| 1261 | acaggtggag tatggtcatc acaaatgaga gacaacatca tcattagtgg |
| | atctacagat |
| 1321 | cggacactca aagtgtggaa tgcagagact ggagaatgta tacacacctt |
| | atatgggcat |
| 1381 | acttccactg tgcgttgtat gcatcttcat gaaaaaagag ttgttagcgg |
| | ttctcgagat |
| 1441 | gccactctta gggtttggga tattgagaca ggccagtgtt tacatgtttt |
| | gatgggtcat |
| 1501 | gttgcagcag tccgctgtgt tcaatatgat ggcaggaggg ttgttagtgg |
| | agcatatgat |
| 1561 | tttatggtaa aggtgtggga tccagagact gaaacctgtc tacacacgtt |
| | gcaggggcat |
| 1621 | actaatagag tctattcatt acagtttgat ggtatccatg tggtgagtgg |
| | atctcttgat |
| 1681 | acatcaatcc gtgtttggga tgtggagaca gggaattgca ttcacacgtt |
| | aacagggcac |
| 1741 | cagtcgttaa caagtggaat ggaactcaaa gacaatattc ttgtctctgg |
| | gaatgcagat |
| 1801 | tctacagtta aaatctggga tatcaaaaca ggacagtgtt tacaaacatt |
| | gcaaggtccc |
| 1861 | aacaagcatc agagtgctgt gacctgttta cagttcaaca agaactttgt |
| | aattaccagc |
| 1921 | tcagatgatg gaactgtaaa actatgggac ttgaaaacgg gtgaatttat |
| | tcgaaaccta |
| 1981 | gtcacattgg agagtggggg gagtggggga gttgtgtggc ggatcagagc |
| | ctcaaacaca |
| 2041 | aagctggtgt gtgcagttgg gagtcggaat gggactgaag aaaccaagct |
| | gctggtgctg |
| 2101 | gactttgatg tggacatgaa gtga |

FBXW7 Protein (Homo sapiens)

| SEQ ID NO: 68 | |
|---|---|
| 1 | mnqellsvgs krrrtggslr gnpsssqvde eqmnrvveee qqqqlrqqee |
| | ehtarngevv |
| 61 | gveprpggqn dsqqgqleen nnrfisvded ssgnqeeqee deehageqde |
| | edeeeeemdq |
| 121 | esddfdqsdd ssredehtht nsvtnsssiv dlpvhqlssp fytkttkmkr |
| | kldhgsevrs |
| 181 | fslgkkpckv seytsttglv pcsatpttfg dlraangqgq qrrritsvqp |
| | ptglqewlkm |
| 241 | fqswsgpekl laldelidsc eptqvkhmmq viepqfqrdf isllpkelal |
| | yvlsflepkd |
| 301 | llqaaqtcry wrilaednll wrekckeegi deplhikrrk vikpgfihsp |
| | wksayirqhr |
| 361 | idtnwrrgel kspkvlkghd dhvitclqfc gnrivsgsdd ntlkvwsavt |
| | gkclrtlvgh |
| 421 | tggvwssqmr dniiisgstd rtlkvwnaet gecihtlygh tstvrcmhlh |
| | ekrvvsgsrd |
| 481 | atlrvwdiet gqclhvlmgh vaavrcvqyd grrvvsgayd fmvkvwdpet |
| | etclhtlqgh |
| 541 | tnrvyslqfd gihvvsgsld tsirvwdvet gncihtltgh qsltsgmelk |
| | dnilvsgnad |
| 601 | stvkiwdikt gqclqtlqgp nkhqsavtcl qfnknfvits sddgtvklwd |
| | lktgefirnl |
| 661 | vtlesggsgg vvwrirasnt klvcavgsrn gteetkllvl dfdvdmk |

TABLE 1
MCR overlap between murine TKO and human T-ALL datasets

| | Mouse | | | | | | Cancer | | | | | |
|---|---|---|---|---|---|---|---|---|---|---|---|---|
| | TKO | | | | | | Genes | Human T-ALL | | | | |
| | | | | | Peak | | or | | | | | Peak |
| MCR # | Cytoband | Start | End | Size (bp) | Ratio | Rec | Candidates | Chr | Start | End | Size (bp) | Ratio |

Amplified MCRs

| | | | | | | | | | | | | |
|---|---|---|---|---|---|---|---|---|---|---|---|---|
| 1 | 4E2 | 153362787 | 154677539 | 1,314,752 | 0.88 | 13 | Dvl1; Ccnl2; | 1 | 1286939.5 | 1536335.5 | 249,396 | 1.11 |
| | | | | | | | Aurkaip1 | | | | | |
| 2 | 10A3 | 18124375 | 22105516 | 3,981,141 | 1.91 | 11 | Myb; Ahi1 | 6 | 135471648.5 | 135829074.5 | 357,426 | 1.07 |
| 3 | 16C4 | 91250715 | 97408345 | 6,157,630 | 1.38 | 21 | Runx1; Ets2; | 21 | 40837575.5 | 42285661.5 | 1,448.086 | 0.95 |
| | | | | | | | Tmprss2; | | | | | |
| | | | | | | | Ripk4; Erg | | | | | |
| 4 | 5G2 | 136128574 | 138413308 | 2,284,734 | 0.87 | 14 | Gnb2; Perq1 | 7 | 99901102.5 | 99949527 | 48,425 | 1.09 |
| 5 | 4A1 | 5601642 | 13568807 | 7,967,165 | 1.00 | 11 | Tox | 8 | 59880732.5 | 60101149.5 | 220,417 | 0.82 |
| 6 | 2B | 29315580 | 31992174 | 2,676,594 | 1.78 | 7 | Set; Fnbp1; | 9 | 130710910.5 | 131134550.5 | 423,640 | 2.06 |
| | | | | | | | Abl1; | | | | | |
| | | | | | | | NUP214 | | | | | |

Deleted MCRs

| | | | | | | | | | | | | |
|---|---|---|---|---|---|---|---|---|---|---|---|---|
| 7 | 11B3-B4 | 68759068 | 72041187 | 3,282,119 | −0.93 | 4 | Trp53; Bcl6b | 17 | 6494426.5 | 7767821.5 | 1,273,395 | −0.76 |
| 8 | 3H4 | 155474073 | 158861389 | 3,387,316 | −0.75 | 3 | Negr1 | 1 | 71919083.5 | 72444137.5 | 525,054 | −0.92 |
| 9 | 15B3.1 | 33212025 | 41060793 | 7,848,768 | −0.93 | 2 | Baalc; Fzd6 | 8 | 104310865.5 | 104499581.5 | 188,716 | −0.93 |
| 10 | 16A1 | 3264231 | 10275117 | 7,010,886 | −0.97 | 21 | Crebbp; C2ta | 16 | 3195168 | 11549999.5 | 8,354,832 | −1.09 |
| 11 | 19C3-D2 | 46457272 | 56116765 | 9,659,493 | −0.77 | 8 | Mxi1 | 10 | 111672720.5 | 112043485.5 | 370,765 | −0.90 |
| 12 | 4E2 | 150778332 | 154677539 | 3,899,207 | −0.83 | 2 | Hes3; | 1 | 5983967.5 | 6318619.5 | 334,652 | −0.85 |
| | | | | | | | RPL22; | | | | | |
| | | | | | | | CHD5 | | | | | |
| 13 | 11A1 | 8844892 | 12372703 | 3,527,811 | −3.73 | 14 | Ikaros | 7 | 49539939.5 | 50229252.5 | 689,313 | −0.75 |
| 14 | 12F2 | 111667310 | 115272402 | 3,605,092 | −1.43 | 9 | Ptprn2 | 7 | 156125925.5 | 158194699.5 | 2,068,774 | −0.84 |
| 15 | 6B1 | 41191601 | 41690238 | 498,637 | −5.48 | 28 | TCRβ | 7 | 141785426.5 | 142078458.5 | 293,032 | −3.07 |
| 16 | 19A | 11295986 | 15610191 | 4,314,205 | −0.77 | 4 | Gnaq | 9 | 77572992.5 | 77916022.5 | 343,030 | −0.76 |
| 17 | 19C1 | 31573449 | 32118682 | 545,233 | −4.48 | 13 | Pten | 10 | 89594719.5 | 90035234.5 | 440,515 | −3.30 |
| 18 | 3E3-F1 | 79297034 | 87003791 | 7,706,757 | −0.93 | 2 | Fbxw7 | 4 | 153078068.5 | 154979435.5 | 1,901,367 | −1.74 |

Each murine TKO MCR with syntenic overlap with an MCR in the human T-ALL dataset is listed, separated by amplification and deletion, along with its chromosomal location (Cytoband/Chr) and base number (Start and End, in Mb).
The minimal size of each MCR is indicated in bp.
Peak ratio refers to the maximal log2 array-CGH ratio for each MCR.
Rec refers to the number of tumors in which the MCR was defined.

TABLE 2
Summary of mutations in human T-ALL cell lines and primary
samples
Each case has been characterized for mutations in NOTCH1, FBXW7
and PTEN. The table shows the breakdown of cell lines and primary
T-ALL samples by two pairwise comparisons NOTCH1 × FBXW7
and NOTCH1 × PTEN. Thus each case appears twice in the table,
once in the FBXW7 column and once in the PTEN column.

| | | FBXW7 | | | |
|---|---|---|---|---|---|
| | | | Mut'd/ | PTEN | |
| | | Wildtype | Del'd* | Wildtype | Mutated |
| Cell lines | | | | | |
| NOTCH1 | Wildtype | 5 | 3 | 7 | 1 |
| | HD only | 1 | 6 | 4 | 3 |
| | PEST only | 3 | 1 | 3 | 1 |
| | HD + PEST | 3 | 1 | 2 | 2 |
| Primary Samples | | | | | |
| NOTCH1 | Wildtype | 12 | 2 | 12 | 2 |
| | HD only | 6 | 7 | 13 | 0 |
| | PEST only | 2 | 1 | 3 | 0 |
| | HD + PEST | 7 | 1 | 8 | 0 |

*mutated or deleted

TABLE 3
Murine TKO tumors used in this study.

| | Genotype | | | | Characterization | | |
|---|---|---|---|---|---|---|---|
| TUMOR | mTerc | Atm | p53 | Surface marker phenotype | aCGH | SKY | Notch1 Status |
| A701 | WT | null | het | nd | yes | yes | |
| KM343 | WT | null | het | CD4+/− CD8+ | yes | yes | |
| CA342 | WT | null | het | mixed CD4+ CD8+ and CD4− | yes | yes | ins CC after 6961A |
| | | | | CD8+ | | | |
| A494 | G0 | null | WT | CD4+ CD8+ | yes | yes | ex34 deletion |
| A934 | G0 | null | ? | nd | yes | yes | |
| A1005 | G0 | null | het | CD4− CD8+ | yes | yes | aa1685 S to C |
| A1252 | G0 | null | het | CD4− CD8+ | yes | yes | ampl/trans? |
| CA373 | G0 | null | ? | nd | yes | yes | |
| CA325 | G0 | null | WT | CD4+ CD8+/− | yes | yes | del6848-6850CTA, ins |
| | | | | | | | GGGG |
| CA318 | G0 | null | ? | nd | yes | no | del 7094A, insCCCCC |
| CA290 | G0 | null | het | CD4− CD8+ | yes | yes | del 7082G, insAA |
| CA235 | G0 | null | het | nd | yes | no | |
| CA250 | G0 | null | het | nd | yes | no | |
| CA371 | G0 | null | het | nd | yes | no | |
| A1118 | G1 | null | het | nd | yes | no | aa1685 S to C |
| A725 | G1 | null | WT | CD4+ CD8+ | yes | yes | del @ nt7260 |
| A933 | G1 | null | het | CD4− CD8+ | yes | no | |
| A1040 | G2 | null | het | CD4− CD8+ | yes | no | |
| A1240 | G2 | null | het | CD4− CD8− | yes | yes | aa1685 S to C |
| A689 | G4 | null | het | CD4+ CD8+ | yes | no | del nt7219-7593 of ORF |
| A785 | G3 | null | WT | CD4+ CD8+ | yes | no | |
| A570 | G3 | null | het | nd | yes | no | |
| A764 | G4 | null | het | nd | yes | no | |
| A543 | G4 | null | het | nd | yes | no | |
| A577 | G4 | null | het | CD4+ CD8+ | yes | yes | ampl/trans? |
| A897 | G4 | null | null | nd | yes | no | |
| A878 | G3 | null | het | Mixed CD4− CD8+ and CD4+ | yes | yes | del @ nt7461 |
| | | | | CD8+ | | | |
| A791 | G3 | null | het | nd | yes | yes | del @ nt7083 |
| A1060 | G3 | null | het | Mixed CD4+ CD8− and CD4+ | yes | yes | aa1683 F to S |
| | | | | CD8+ | | | |
| A895 | G4 | null | null | CD4+CD8+ | yes | yes | ampl/trans? |
| A684 | G4 | null | het | nd | yes | yes | |
| A1052 | G3 | null | WT | nd | yes | yes | ampl/trans? |
| CA456 | G0 | WT | null | CD4+/− CD8+ | yes | no | amplification |
| CA427 | G0 | het | null | CD4+/− CD8+ | yes | no | amplification |
| KM168 | G0 | WT | null | nd | yes | no | |

TABLE 4A
T-ALL cell lines

| | | | | | Array- |
|---|---|---|---|---|---|
| Sample | Type | Age | Sex | Sequenced* | CGH* |
| BE-13 | cell line | 4 | F | yes | yes |
| CCRF- | cell line | 4 | F | yes | yes |
| CEM | | | | | |
| CML-T1 | cell line | 36 | F | yes | no |
| CTV-1 | cell line | 40 | F | yes | no |
| DND41 | cell line | 13 | M | yes | yes |
| DU528 | cell line | 16 | M | yes | yes |
| HBP-ALL | cell line | 14 | M | yes | yes |
| J-RT3-T3-5 | cell line | 14 | M | yes | no |
| KARPAS- | cell line | 2 | M | yes | no |
| 45 | | | | | |
| KE-37 | cell line | 27 | M | yes | no |
| KopTK1 | cell line | pediatric | | yes | yes |
| LOUCY | cell line | 38 | F | yes | yes |
| ML-2 | cell line | 26 | M | yes | no |
| MOLT-13 | cell line | 2 | F | yes | yes |
| MOLT-16 | cell line | 5 | F | yes | yes |
| MOLT-4 | cell line | 19 | M | yes | yes |
| P12- | cell line | 7 | M | yes | no |
| ICHIKAWA | | | | | |
| PF-382 | cell line | 6 | F | yes | yes |
| RPMI- | cell line | 16 | F | yes | yes |
| 8402 | | | | | |
| SupT11 | cell line | 74 | M | yes | yes |
| SupT13 | cell line | pediatric | | yes | yes |
| SupT7 | cell line | pediatric | | yes | yes |
| TALL-1 | cell line | 28 | M | yes | yes |
| Jurkat | cell line | 14 | M | no | yes |
| ALL-SIL | cell line | 17 | M | no | yes |

*indicates whether samples were used for either aCGH and/or re-squencing efforts

TABLE 4B
T-ALL tumors profiled by array-CGH*

| Sample | Type | Age | Sex |
|---|---|---|---|
| XC018-PB | clinical | 10 | M |
| TL037 | clinical | 11 | M |
| MD108 | clinical | 15 | F |
| CO155 | clinical | 15 | F |
| RS128 | clinical | 4 | F |
| MP496 | clinical | 13 | F |
| JB238-PB | clinical | 4 | M |
| BN066- | normal | | |
| D28 | remission | | |

*Clinical samples profiled by aCGH; samples not subjected to re-sequencing

TABLE 4C
Clinical specimens Sequenced*

| Sample | Type | Age | Sex |
|---|---|---|---|
| PD2716a | clinical | 17 | F |
| PD2717a | clinical | 19 | M |
| PD2718a | clinical | 16 | M |
| PD2719a | clinical | 14 | M |
| PD2720a | clinical | 9 | M |
| PD2721a | clinical | 33 | M |
| PD2722a | clinical | 26 | F |
| PD2724a | clinical | 55 | M |
| PD2725a | clinical | 46 | M |
| PD2726a | clinical | 25 | M |
| PD2727a | clinical | 39 | M |
| PD2728a | clinical | 24 | M |
| PD2729a | clinical | 42 | M |
| PD2730a | clinical | 26 | F |
| PD2731a | clinical | 19 | M |
| PD2732a | clinical | 46 | F |
| PD2733a | clinical | 21 | M |
| PD2734a | clinical | 37 | F |
| PD2735a | clinical | 27 | M |
| PD2736a | clinical | 16 | M |
| PD2737a | clinical | 36 | M |
| PD2738a | clinical | 8 | M |
| PD2739a | clinical | 31 | M |
| PD2740a | clinical | 35 | M |
| PD2741a | clinical | 37 | M |
| PD2742a | clinical | 44 | M |
| PD2743a | clinical | 2 | M |
| PD2744a | clinical | 25 | M |
| PD2745a | clinical | 39 | F |
| PD2746a | clinical | 32 | M |
| PD2747a | clinical | 32 | M |
| PD2748a | clinical | 7 | M |
| PD2749a | clinical | 19 | M |
| PD2750a | clinical | 44 | M |
| PD2751a | clinical | 17 | M |
| PD2752a | clinical | 30 | M |
| PD2753a | clinical | 15 | M |
| PD2754a | clinical | 17 | M |

*Clinical specimens used for re-sequencing; samples not profiled by aCGH

TABLE 5
List of 160 MCRs defined in TKO genomes

| | | Position | | Cytobands | | Peak | | | |
|---|---|---|---|---|---|---|---|---|---|
| mid | chn | start | end | start | end | Ratio | Recurrence | Width (bp) | # of Genes |
| 141 | 1 | 1.05E+08 | 1.06E+08 | 1qE2.1 | 1qE2.1 | 1.044 | 9 | 1,110,166 | 5 |
| 68 | 1 | 1.28E+08 | 1.28E+08 | 1qE3 | 1qE3 | 0.945 | 10 | 362,010 | 5 |
| 67 | 1 | 1.28E+08 | 1.28E+08 | 1qE3 | 1qE3 | 2.099 | 13 | 142,785 | 4 |
| 70 | 1 | 1.31E+08 | 1.36E+08 | 1qE4 | 1qE4 | 0.888 | 10 | 5,086,790 | 100 |
| 69 | 1 | 1.36E+08 | 1.39E+08 | 1qE4 | 1qE4 | 0.888 | 11 | 2,430,212 | 14 |
| 149 | 1 | 1.5E+08 | 1.5E+08 | 1qG1 | 1qG1 | 1.041 | 13 | 31,937 | 2 |
| 86 | 2 | 18256403 | 19011398 | 2qA3 | 2qA3 | 1.552 | 11 | 754,995 | 7 |
| 85 | 2 | 26220146 | 26426743 | 2qA3 | 2qA3 | 2.521 | 13 | 206,597 | 10 |
| 87 | 2 | 29076116 | 29113534 | 2qB | 2qB | 0.946 | 7 | 37,418 | 1 |
| 88 | 2 | 29315580 | 31992174 | 2qB | 2qB | 1.782 | 7 | 2,676,594 | 60 |
| 89 | 2 | 32141443 | 33152477 | 2qB | 2qB | 1.258 | 6 | 1,011,034 | 35 |
| 5 | 2 | 86526803 | 87088323 | 2qD | 2qD | 0.937 | 5 | 561,520 | 33 |
| 105 | 2 | 1.29E+08 | 1.31E+08 | 2qF1 | 2qF1 | 1.191 | 6 | 2,182,234 | 49 |
| 73 | 2 | 1.49E+08 | 1.57E+08 | 2qG3 | 2qH1 | 0.907 | 7 | 8,124,884 | 176 |
| 72 | 2 | 1.57E+08 | 1.58E+08 | 2qH1 | 2qH1 | 0.898 | 8 | 89,827 | 2 |
| 42 | 2 | 1.78E+08 | 1.78E+08 | 2qH4 | 2qH4 | 1.043 | 5 | 56,696 | 4 |
| 45 | 4 | 5601642 | 13568807 | 4qA1 | 4qA1 | 1.001 | 11 | 7,967,165 | 50 |
| 48 | 4 | 43960797 | 44207047 | 4qB1 | 4qB1 | 0.855 | 14 | 246,250 | 2 |
| 49 | 4 | 46581252 | 48074866 | 4qB1 | 4qB1 | 0.966 | 15 | 1,493,614 | 12 |
| 46 | 4 | 59204015 | 59696580 | 4qB3 | 4qB3 | 1.312 | 15 | 492,565 | 6 |
| 47 | 4 | 61574346 | 61615586 | 4qB3 | 4qB3 | 1.759 | 16 | 41,240 | 4 |
| 50 | 4 | 67845996 | 69605630 | 4qC1 | 4qC2 | 0.962 | 15 | 1,759,634 | 6 |
| 107 | 4 | 73573051 | 82835399 | 4qC3 | 4qC3 | 0.844 | 15 | 9,262,348 | 24 |
| 8 | 4 | 1.06E+08 | 1.06E+08 | 4qC7 | 4qC7 | 0.928 | 16 | 121,051 | 4 |
| 6 | 4 | 1.47E+08 | 1.51E+08 | 4qE2 | 4qE2 | 0.821 | 15 | 4,128,560 | 67 |
| 7 | 4 | 1.53E+08 | 1.55E+08 | 4qE2 | 4qE2 | 0.881 | 13 | 1,314,752 | 53 |
| 118 | 5 | 29600288 | 31438940 | 5qB1 | 5qB1 | 0.882 | 11 | 1,838,652 | 30 |
| 75 | 5 | 44135455 | 44256743 | 5qB3 | 5qB3 | 1.188 | 12 | 121,288 | 2 |
| 9 | 5 | 85392518 | 85451062 | 5qE1 | 5qE1 | 0.882 | 11 | 58,544 | 2 |
| 14 | 5 | 1.02E+08 | 1.02E+08 | 5qE5 | 5qE5 | 0.841 | 9 | 185,602 | 3 |
| 12 | 5 | 1.05E+08 | 1.08E+08 | 5qE5 | 5qF | 1.956 | 10 | 2,704,253 | 33 |
| 15 | 5 | 1.13E+08 | 1.15E+08 | 5qF | 5qF | 0.839 | 12 | 2,276,889 | 54 |
| 11 | 5 | 1.35E+08 | 1.36E+08 | 5qG2 | 5qG2 | 1.472 | 13 | 905,844 | 15 |
| 13 | 5 | 1.36E+08 | 1.38E+08 | 5qG2 | 5qG2 | 0.867 | 14 | 2,284,734 | 75 |
| 10 | 5 | 1.48E+08 | 1.5E+08 | 5qG3 | 5qG3 | 0.958 | 15 | 1,707,628 | 22 |
| 120 | 6 | 98525054 | 1.03E+08 | 6qD3 | 6qD3 | 1.417 | 1 | 4,114,423 | 14 |
| 121 | 8 | 30677625 | 34627880 | 8qA3 | 8qA4 | 0.752 | 6 | 3,950,255 | 31 |
| 111 | 8 | 74189294 | 74204190 | 8qC1 | 8qC1 | 0.895 | 5 | 14,896 | 2 |
| 17 | 9 | 29333867 | 32712352 | 9qA4 | 9qA4 | 1.776 | 12 | 3,378,485 | 21 |
| 20 | 9 | 44813433 | 45348832 | 9qA5.2 | 9qA5.2 | 0.850 | 7 | 535,399 | 15 |
| 16 | 9 | 46329619 | 47484838 | 9qA5.3 | 9qA5.3 | 1.555 | 15 | 1,155,219 | 5 |
| 123 | 9 | 53345703 | 54059125 | 9qA5.3 | 9qA5.3 | 0.752 | 4 | 713,422 | 14 |
| 124 | 9 | 56482435 | 56638553 | 9qB | 9qB | 0.887 | 5 | 156,118 | 2 |
| 125 | 9 | 59310802 | 59590013 | 9qB | 9qB | 0.752 | 5 | 279,211 | 3 |
| 76 | 10 | 18124375 | 22105516 | 10qA3 | 10qA3 | 1.914 | 11 | 3,981,141 | 37 |
| 77 | 10 | 39797713 | 39991041 | 10qB1 | 10qB1 | 0.933 | 10 | 193,328 | 4 |
| 114 | 10 | 75079313 | 75286215 | 10qC1 | 10qC1 | 0.918 | 5 | 206,902 | 5 |
| 127 | 10 | 93180073 | 99904446 | 10qC2 | 10qD1 | 0.854 | 5 | 6,724,373 | 56 |
| 104 | 10 | 1.27E+08 | 1.27E+08 | 10qD3 | 10qD3 | 0.854 | 11 | 299,603 | 18 |
| 143 | 11 | 3094931 | 4168597 | 11qA1 | 11qA1 | 0.757 | 2 | 1,073,666 | 33 |
| 100 | 11 | 32195496 | 36843135 | 11qA4 | 11qA5 | 0.872 | 7 | 4,647,639 | 29 |
| 101 | 11 | 40488257 | 44855717 | 11qA5 | 11qB1.1 | 0.898 | 6 | 4,367,460 | 23 |
| 102 | 11 | 45787203 | 48749988 | 11qB1.1 | 11qB1.2 | 0.932 | 7 | 2,962,785 | 32 |
| 128 | 11 | 1.17E+08 | 1.18E+08 | 11qE2 | 11qE2 | 0.755 | 7 | 822,168 | 21 |
| 129 | 11 | 1.18E+08 | 1.19E+08 | 11qE2 | 11qE2 | 0.808 | 8 | 726,438 | 14 |
| 78 | 12 | 38086004 | 46238385 | 12qB1 | 12qB3 | 0.981 | 11 | 8,152,381 | 20 |
| 79 | 12 | 47390537 | 52540991 | 12qB3 | 12qC1 | 1.466 | 10 | 5,150,454 | 44 |
| 80 | 12 | 55790095 | 55837560 | 12qC1 | 12qC1 | 0.942 | 11 | 47,465 | 5 |
| 51 | 12 | 75416967 | 76481214 | 12qC3 | 12qC3 | 0.828 | 11 | 1,064,247 | 17 |
| 53 | 13 | 3825590 | 10409879 | 13qA1 | 13qA1 | 1.243 | 3 | 6,584,289 | 34 |
| 54 | 13 | 23330778 | 24380522 | 13qA3.1 | 13qA3.1 | 1.039 | 1 | 1,049,744 | 17 |
| 56 | 13 | 46322053 | 47532316 | 13qA5 | 13qA5 | 0.976 | 1 | 1,210,263 | 10 |
| 25 | 13 | 99644459 | 1.01E+08 | 13qD1 | 13qD1 | 1.195 | 2 | 1,193,251 | 13 |
| 26 | 13 | 1.03E+08 | 1.1E+08 | 13qD2.1 | 13qD2.2 | 1.811 | 2 | 6,946,446 | 47 |
| 57 | 14 | 40458276 | 41162221 | 14qB | 14qB | 2.846 | 25 | 703,945 | 9 |
| 58 | 14 | 41747861 | 44316485 | 14qC1 | 14qC1 | 2.997 | 24 | 2,568,624 | 30 |
| 59 | 14 | 46887800 | 48318364 | 14qC1 | 14qC1 | 1.980 | 22 | 1,430,564 | 63 |
| 62 | 14 | 61322898 | 67876948 | 14qD1 | 14qD2 | 0.957 | 15 | 6,554,050 | 72 |
| 60 | 14 | 73311656 | 73991889 | 14qD3 | 14qD3 | 1.042 | 14 | 680,233 | 11 |
| 61 | 14 | 81055230 | 81965738 | 14qE1 | 14qE1 | 2.163 | 14 | 910,508 | 2 |
| 64 | 14 | 90605302 | 91070049 | 14qE2.1 | 14qE2.1 | 2.038 | 14 | 464,747 | 1 |
| 65 | 14 | 92428111 | 93598116 | 14qE2.1 | 14qE2.1 | 1.919 | 14 | 1,170,005 | 5 |
| 66 | 14 | 94810852 | 97523812 | 14qE2.2 | 14qE2.3 | 1.526 | 14 | 2,712,960 | 10 |
| 63 | 14 | 1.16E+08 | 1.17E+08 | 14qE5 | 14qE5 | 0.982 | 16 | 966,790 | 12 |
| 28 | 15 | 4902782 | 6271853 | 15qA1 | 15qA1 | 1.578 | 17 | 1,369,071 | 9 |
| 30 | 15 | 23144859 | 32967402 | 15qA2 | 15qB3.1 | 1.233 | 18 | 9,822,543 | 41 |
| 29 | 15 | 54425386 | 63790043 | 15qD1 | 15qD1 | 1.498 | 20 | 9,364,657 | 68 |
| 27 | 15 | 95452330 | 1.03E+08 | 15qF1 | 15qF3 | 1.028 | 20 | 7,131,911 | 192 |
| 33 | 16 | 42899450 | 43217357 | 16qB4 | 16qB4 | 0.988 | 12 | 317,907 | 5 |
| 31 | 16 | 48142711 | 55198270 | 16qB5 | 16qC1.1 | 0.989 | 13 | 7,055,559 | 27 |
| 32 | 16 | 55961953 | 56077653 | 16qC1.1 | 16qC1.1 | 0.913 | 13 | 115,700 | 4 |
| 34 | 16 | 74969013 | 76202427 | 16qC3.1 | 16qC3.1 | 1.030 | 16 | 1,233,414 | 4 |
| 83 | 16 | 83801341 | 84228153 | 16qC3.3 | 16qC3.3 | 1.293 | 18 | 426,812 | 7 |
| 82 | 16 | 86584797 | 87663238 | 16qC3.3 | 16qC3.3 | 1.178 | 18 | 1,078,441 | 11 |
| 81 | 16 | 91250715 | 97408345 | 16qC4 | 16qC4 | 1.378 | 21 | 6,157,630 | 53 |
| 36 | 17 | 11029895 | 11172149 | 17qA1 | 17qA1 | 0.997 | 5 | 142,254 | 2 |
| 35 | 17 | 12996985 | 13092851 | 17qA1 | 17qA1 | 1.423 | 9 | 95,866 | 6 |
| 37 | 17 | 28187374 | 28772915 | 17qA3.3 | 17qA3.3 | 1.272 | 14 | 585,541 | 4 |
| 40 | 17 | 31307004 | 32045121 | 17qB1 | 17qB1 | 0.920 | 6 | 738,117 | 46 |
| 39 | 17 | 33888591 | 33972790 | 17qB1 | 17qB1 | 1.647 | 6 | 84,199 | 2 |
| 41 | 17 | 48468702 | 54249820 | 17qC | 17qC | 0.834 | 4 | 5,781,118 | 65 |
| 84 | 18 | 44249076 | 44496478 | 18qB3 | 18qB3 | 0.907 | 3 | 247,402 | 6 |
| 92 | 19 | 3307019 | 4813998 | 19qA | 19qA | 1.091 | 3 | 1,506,979 | 64 |
| 93 | 19 | 8172318 | 9587961 | 19qA | 19qA | 1.242 | 4 | 1,415,643 | 23 |
| 94 | 19 | 9746944 | 12276560 | 19qA | 19qA | 1.449 | 4 | 2,529,616 | 107 |
| 103 | 19 | 38219064 | 38791620 | 19qC3 | 19qC3 | 0.763 | 3 | 572,556 | 7 |
| 95 | 19 | 43353084 | 43585182 | 19qC3 | 19qC3 | 0.961 | 2 | 232,098 | 5 |
| 96 | 19 | 44700687 | 44972460 | 19qC3 | 19qC3 | 1.023 | 2 | 271,773 | 3 |
| 97 | 19 | 45365601 | 46170449 | 19qC3 | 19qC3 | 0.876 | 2 | 804,848 | 20 |
| 140 | 19 | 54723418 | 54846569 | 19qD2 | 19qD2 | 0.898 | 2 | 123,151 | 5 |
| 98 | 19 | 59483972 | 60620320 | 19qD3 | 19qD3 | 1.339 | 3 | 1,136,348 | 13 |
| 221 | 1 | 29038485 | 29089894 | 1qA5 | 1qA5 | −1.092 | 1 | 51,409 | 2 |
| 193 | 2 | 26426743 | 30018849 | 2qA3 | 2qB | −0.884 | 1 | 3,592,106 | 70 |
| 209 | 2 | 33052450 | 33773524 | 2qB | 2qB | −0.948 | 3 | 721,074 | 9 |
| 177 | 2 | 1.67E+08 | 1.68E+08 | 2qH3 | 2qH3 | −1.072 | 2 | 694,349 | 12 |
| 194 | 2 | 1.69E+08 | 1.7E+08 | 2qH3 | 2qH3 | −0.871 | 2 | 548,165 | 3 |
| 195 | 2 | 1.72E+08 | 1.72E+08 | 2qH3 | 2qH3 | −0.786 | 3 | 64,794 | 2 |
| 196 | 3 | 53093840 | 57750461 | 3qC | 3qD | −1.000 | 3 | 4,656,621 | 39 |
| 237 | 3 | 72799409 | 73392410 | 3qE3 | 3qE3 | −0.841 | 3 | 593,001 | 2 |
| 191 | 3 | 78211040 | 78797254 | 3qE3 | 3qE3 | −0.841 | 5 | 586,214 | 4 |
| 197 | 3 | 79297034 | 87003791 | 3qE3 | 3qF1 | −0.932 | 2 | 7,706,757 | 56 |
| 186 | 3 | 1.55E+08 | 1.59E+08 | 3qH4 | 3qH4 | −0.752 | 3 | 3,387,316 | 13 |
| 198 | 4 | 1.11E+08 | 1.12E+08 | 4qD1 | 4qD1 | −0.921 | 2 | 654,234 | 8 |
| 212 | 4 | 1.37E+08 | 1.37E+08 | 4qD3 | 4qD3 | −1.153 | 3 | 217,944 | 2 |
| 224 | 4 | 1.51E+08 | 1.55E+08 | 4qE2 | 4qE2 | −0.834 | 2 | 3,899,207 | 78 |
| 150 | 5 | 21196088 | 21737788 | 5qA3 | 5qA3 | −1.044 | 2 | 541,700 | 1 |
| 151 | 6 | 41191601 | 41690238 | 6qB1 | 6qB1 | −5.480 | 28 | 498,637 | 21 |
| 235 | 6 | 73593839 | 80776018 | 6qC1 | 6qC3 | −0.787 | 3 | 7,182,179 | 20 |
| 229 | 7 | 1.26E+08 | 1.26E+08 | 7qF3 | 7qF3 | −1.048 | 2 | 106,584 | 3 |
| 225 | 7 | 1.37E+08 | 1.4E+08 | 7qF5 | 7qF5 | −0.895 | 3 | 2,633,930 | 38 |
| 213 | 8 | 76735909 | 76808515 | 8qC1 | 8qC1 | −0.881 | 4 | 72,606 | 2 |
| 201 | 10 | 3207257 | 9357502 | 10qA1 | 10qA1 | −0.976 | 1 | 6,150,245 | 38 |
| 183 | 11 | 8844892 | 12372703 | 11qA1 | 11qA1 | −3.730 | 14 | 3,527,811 | 18 |
| 184 | 11 | 16565410 | 17157549 | 11qA2 | 11qA2 | −0.947 | 7 | 592,139 | 11 |
| 230 | 11 | 25513879 | 33407529 | 11qA3.2 | 11qA4 | −0.916 | 5 | 7,893,650 | 61 |
| 226 | 11 | 44209892 | 44304867 | 11qB1.1 | 11qB1.1 | −0.935 | 5 | 94,975 | 2 |
| 189 | 11 | 68759068 | 72041187 | 11qB3 | 11qB4 | −0.932 | 4 | 3,282,119 | 125 |
| 218 | 11 | 92848956 | 93404029 | 11qD | 11qD | −0.927 | 3 | 555,073 | 2 |
| 227 | 12 | 93606364 | 93916807 | 12qE | 12qE | −0.870 | 3 | 310,443 | 3 |
| 154 | 12 | 96250531 | 96496843 | 12qE | 12qE | −0.895 | 5 | 246,312 | 4 |
| 153 | 12 | 98783592 | 1.04E+08 | 12qE | 12qF1 | −1.602 | 15 | 5,234,816 | 66 |
| 155 | 12 | 1.12E+08 | 1.15E+08 | 12qF2 | 12qF2 | −1.427 | 9 | 3,605,092 | 25 |
| 179 | 13 | 18627216 | 18826113 | 13qA2 | 13qA2 | −3.237 | 12 | 198,897 | 1 |
| 180 | 13 | 37254725 | 37524185 | 13qA3.3 | 13qA3.3 | −0.986 | 9 | 269,460 | 3 |
| 181 | 13 | 48176346 | 50100290 | 13qA5 | 13qA5 | −1.190 | 9 | 1,923,944 | 31 |
| 156 | 13 | 97118503 | 98856406 | 13qD1 | 13qD1 | −0.875 | 8 | 1,737,903 | 2 |
| 203 | 13 | 1.14E+08 | 1.15E+08 | 13qD2.3 | 13qD2.3 | −0.913 | 8 | 405,653 | 1 |
| 157 | 14 | 24250524 | 24460588 | 14qA3 | 14qA3 | −1.187 | 6 | 210,064 | 6 |
| 240 | 14 | 44277623 | 45455380 | 14qC1 | 14qC1 | −0.833 | 4 | 1,177,757 | 22 |
| 214 | 14 | 46642257 | 46906069 | 14qC1 | 14qC1 | −2.581 | 7 | 263,812 | 7 |
| 215 | 14 | 46983329 | 47000386 | 14qC1 | 14qC1 | −0.874 | 3 | 17,057 | 3 |
| 158 | 14 | 47563191 | 48727495 | 14qC1 | 14qC1 | −4.918 | 20 | 1,164,304 | 41 |
| 204 | 14 | 63792812 | 64013139 | 14qD1 | 14qD1 | −1.202 | 8 | 220,327 | 4 |
| 234 | 14 | 1.1E+08 | 1.19E+08 | 14qE4 | 14qE5 | −0.990 | 3 | 8,712,984 | 54 |
| 205 | 15 | 3059822 | 10112117 | 15qA1 | 15qA1 | −0.999 | 2 | 7,052,295 | 52 |
| 206 | 15 | 33212025 | 41060793 | 15qB3.1 | 15qB3.1 | −0.935 | 2 | 7,848,768 | 59 |
| 228 | 15 | 91904361 | 93343014 | 15qE3 | 15qE3 | −0.997 | 2 | 1,438,653 | 9 |
| 159 | 16 | 3264231 | 10275117 | 16qA1 | 16qA1 | −0.971 | 21 | 7,010,886 | 74 |
| 160 | 16 | 15680940 | 16190296 | 16qA2 | 16qA2 | −0.779 | 10 | 509,356 | 16 |
| 161 | 16 | 17292404 | 18721258 | 16qA3 | 16qA3 | −0.958 | 11 | 1,428,854 | 35 |
| 162 | 16 | 19589196 | 21020820 | 16qA3 | 16qB1 | −0.892 | 9 | 1,431,624 | 20 |
| 208 | 18 | 11094974 | 11165506 | 18qA1 | 18qA1 | −0.791 | 3 | 70,532 | 2 |
| 239 | 19 | 11295986 | 15610191 | 19qA | 19qA | −0.773 | 4 | 4,314,205 | 106 |
| 164 | 19 | 26046566 | 28527676 | 19qC1 | 19qC1 | −0.851 | 7 | 2,481,110 | 21 |
| 165 | 19 | 28881381 | 29036087 | 19qC1 | 19qC1 | −0.851 | 5 | 154,706 | 4 |
| 163 | 19 | 31573449 | 32118682 | 19qC1 | 19qC1 | −4.479 | 13 | 545,233 | 8 |
| 166 | 19 | 33295876 | 35125747 | 19qC1 | 19qC2 | −3.887 | 6 | 1,829,871 | 22 |
| 187 | 19 | 36783412 | 41421335 | 19qC2 | 19qC3 | −0.951 | 6 | 4,637,923 | 62 |
| 220 | 19 | 46457272 | 56116765 | 19qC3 | 19qD2 | −0.768 | 8 | 9,659,493 | 65 |
| 185 | 19 | 59063578 | 59662870 | 19qD3 | 19qD3 | −0.768 | 9 | 599,292 | 3 |

TABLE 6
Mutations in human T-ALL cell lines and primary samples.

| Sample | FBXW7 mutation | NOTCH1 mutation | PTEN mutation |
|---|---|---|---|
| BE-13 | Homozygous Deletion | Hom c.4802T > C p.L1601P | |
| CCRF-CEM | Het c.1393C > T p.R465C | Het c.4784insCGCGCCTTCCCCACAACAGCTCCTTCCACTTCCTGC | |
| | | p.R1595 > PRLPHNSSSHFL | |
| CML-T1 | Het c.1394G > A p.R465H | | |
| CTV-1 | Het c.1513C > T p.R505C | Het c.7571C > A p.S2524* | |
| DND41 | | Hom c.4781T > C p.L1594P | |
| DU528 | Het c.1394G > A p.R465H | | |
| HBP-ALL | Het c.1580A > G p.D527G | Het c.4724T > C p.L1575P, Het c.7329insGGGCCGTGGACG | |
| | | p.D2443fs*39 | |
| J-RT3-T3-5 | Het c.1513C > T p.R505C | | Het c.696_697 > |
| | | | GGCCCATGG p.R233fs*11 |
| KARPAS-45 | Het c.1513C > T p.R505C | Het c.5129T > C p.L1710P | Hom c.1000C > T p.R334* |
| KE-37 | | Het c.7378C > T p.Q2460* | |
| KopTK1 | | Het c.4802T > C p.L1601P, Het c.7544_7545delCT p.P2515fs*4 | |
| LOUCY | | | |
| ML-2 | | Het c.7544_7545delCT p.P2515fs*4 | |
| MOLT-13 | Het c.1394G > A p.R465H | Het c.5036T > C p.L1679P | |
| MOLT-16 | | | |
| MOLT-4 | | Het c.7544_7545delCT p.P2515fs*4 | Hom c.797delA p.K266fs*9 |
| P12- | Hom c.1513C > T p.R505C | Het c.5165ins- | Hom c.818G > A p.W273* |
| | | CCCGGTTGGGCAGCCTCAACATCCCCTACAAGATCGAGGCCG | |
| ICHIKAWA | | p.V1722 > ARWGSLNIPYLIEA | |
| PF-382 | | Het c.4724T > C p.L1575P, Het c.7480insGCCTCTTAGCT p.P2494fs*3 | Hom Exon 5 + 2 ins GCCG p.? |
| RPMI-8402 | Hom c.1394G > | Het c.4754insCCGTGGAGCTGATGCCGCCGGAGC | Het c.477G > T p.R159S, Het |
| | A p.R465H | p.Q1585 > PVELMPPE | c.702_703insCCCCCGGCCC |
| | | | p.D235fs*10 |
| SupT11 | | | |
| SupT13 | | | |
| SupT7 | | Het c.4778insGGGTGC p.F1593 > LGA, Het c.7285insGC p.H2429fs*8 | Het c.699_700insAAGG |
| | | | p.E234fs*9 |
| TALL-1 | | | |
| PD2716a | | | |
| PD2717a | | Het c.4802T > C p.L1601P, Het c.7472insAA p.Y2491fs*1 | |
| PD2718a | | | |
| PD2719a | | Het c.4757T > C p.L1586P, Het c.7331insGGGCATC p.V2444fs*37 | |
| PD2720a | Het c.1513C > T p.R505C | Het c.7253C > T p.P2418L | |
| PD2721a | | Het c.5036T > A p.L16797Q | |
| PD2722a | Het c.1393C > T p.R465C | Het c.4781T > C p.L1594P, Het c.7333C > T p.Q2445* | |
| PD2724a | | Het c.4781T > C p.L1594P | |
| PD2725a | | Het c.4780insTTCGATA p.L1594_R1595 > FDR | |
| PD2726a | | | |
| PD2727a | Het c.1436G > T p.R479L | Het c.4844insTGTGCCG p.Q1615_F1618 > LCR | |
| PD2728a | | | |
| PD2729a | Het c.1268G > T p.G423V | Het c.4751insGTACCCACCCTAAGG p.E1584insGTHPKE | |
| PD2730a | | | Het c.697_698insCACGCTA |
| | | | p.R233fs*3 |
| PD2731a | | | |
| PD2732a | Het c.1393C > T p.R465C | Het c.4858_4859 > CCAGGGT p.Y1620 > PGS | |
| PD2733a | | Het c.5164insCCCCCGGGCAGT p.V1722 > PPGSL | |
| PD2734a | Het. c.1436G > A p.R479Q | Het c.4802T > C p.L1601P | |
| PD2735a | | Het c.4757T > C p.L1586P, Het c.7544_7545delCT p.P2515fs*4 | |
| PD2736a | | | |
| PD2737a | Het c.1393C > T p.R465C | Het c.4776_8delCTT 4776insGAC p.H1592Q F1593T | |
| PD2738a | | Het c.7478insCCCTTGACAGGC p.V2495* | |
| PD2739a | | | |
| PD2740a | Het c.1393C > T p.R465C | Het c.4852_4854delTTC p.F1618del | |
| PD2741a | | Het c.4790T > A p.L1597H | |
| PD2742a | | Het c.5025insGGG p.S1675_I1676insG, | |
| | | Het c.7330insAGGAAAAG p.V2444fs*37 | |
| PD2743a | | | |
| PD2744a | | Het c.4724T > C p.L1575P, Het c.4757T > C p.L1586P, Het c.7390delG | |
| | | p.A2464fs*13 | |
| PD2745a | | Het c.4850T > A p.I1617N, Het c.7305insGGGTG p.S2436fs*2 | |
| PD2746a | Het c.1393C > T p.R465C | Het c.4779insGTCGCC p.L1594 > VA | |
| PD2747a | | Het c.4771insCCA p.F1591 > SI, Het c.7538C > T p.P2513L | |
| PD2748a | | Het c.7372insTAGGGGTTA p.L2458fs*1 | |
| PD2749a | | | |
| PD2750a | | | |
| PD2751a | | | |
| PD2752a | Het Exon 7 + 1G > AA p.? | | |
| PD2753a | | | Het c.694 > GGGAGG |
| | | | p.R232fs*25 |
| PD2754a | Het c.2001insG | | |
| | p.S668fs*26 | | |

TABLE 7
List of known cancer genes mapped to syntenic MCRs in TKO tumors

| | | |
|---|---|---|
| Gene | | Gene |
| Symbols | Gene Symbols | Name |

Oncogenes

| | | |
|---|---|---|
| Myc | myelocytomatosis oncogene | 29 |
| Btg1 | B-cell translocation gene 1, anti-proliferative | 127 |
| Set | SET translocation | 88 |
| Fnbp1 | formin binding protein 1 | 88 |
| Abl1 | v-abl Abelson murine leukemia oncogene 1 | 88 |
| Nup214 | nucleoporin 214 | 88 |
| (BC039282) | | |
| Notch1 | Notch gene homolog 1 | 85 |
| Cdk4 | cyclin-dependent kinase 4 | 104 |
| Ddit3 | DNA-damage inducible transcript 3 | 104 |
| Bcr | breakpoint cluster region homolog | 114 |
| Patz1 | POZ (BTB) and AT hook containing zinc finger 1 | 143 |
| (Zfp278) | | |
| Tpr | translocated promoter region | 149 |
| Rpl22 | ribosomal protein L22 | 6 |
| Nr4a3 | nuclear receptor subfamily 4, group A, member 3 | 49 |
| Mll1(Mll) | myeloid/lymphoid or mixed-lineage leukemia 1 | 20 |
| Gphn | gephyrin | 51 |
| Fli1 | Friend leukemia integration 1 | 17 |

Tumor Suppressors

| | | |
|---|---|---|
| Crebbp | CREB binding protein | 159 |
| Trp53 | transformation related protein 53 | 189 |
| Pten | phosphatase and tensin homolog | 163 |
| Fbxw7 | F-box and WD-40 domain protein 7, | 197 |
| | archipelago homolog (Drosophila) | |
| Npm1 | nucleophosmin 1 | 230 |
| Fas | Fas (TNF receptor superfamily member) | 166 |
| (Tnfrsf6) | | |
| Tsc1 | tuberous sclerosis 1 | 193 |

TABLE 8
primers used for real-time PCR

| | alternative | | |
|---|---|---|---|
| primer | name | sequence | COMMENT |
| D19MIT13A | | TCTGGCACAAAGAGTTCGTG (SEQ ID NO: 69) | PAPSS2 gene |
| D19MIT13B | | CTTTTGCAGGAGCAGGTAGG (SEQ ID NO: 70) | |
| RM120 | AW107648 | AACAGGATATGTTTCTTGGCG (SEQ ID NO: 71) | ATAD1 |
| RM121 | | GGGTTATAGATTGCGGGAGA (SEQ ID NO: 72) | |
| RM127 | | CAGCCGCTGCGAGGATTATCCGTCTTC (SEQ ID | PTEN exon 1 |
| | | NO: 73) | |
| RM128 | | GCGGTCGCTGATGCCCCTCGCTCTG (SEQ ID | |
| | | NO: 74) | |
| RM122 | PMC270016P1 | AAAAGTTCCCCTGCTGATGATTTGT (SEQ ID NO: | Between PTEN exon 5&6 |
| | | 75) | |
| RM123 | | TGTTTTTGACCAATTAAAGTAGGCTGTG (SEQ ID | |
| | | NO: 76) | |
| 119211 FOR | | TGCAGTATAGAGCGTGCAGA (SEQ ID NO: 77) | PTEN EXON 8 |
| 119211 REV | | AGTATCGGTTGGCCTTGTCT (SEQ ID NO: 78) | |

TABLE 9
NCBI accession and reference numbers for cancer genes or
candidate cancer genes listed in Table 1

| | Murine mRNA NM | Murine Entrez | Human Gene |
|---|---|---|---|
| Gene Name | designation | Gene ID | ID |
| Mm Dvl1 | NM_010091 | 13542 | 1855 |
| ccnl2 | NM_207678 | 56036 | 81669 |
| aurkaip1 | NM_025338 | 66077 | 54998 |
| myb | NM_010848 | 17863 | 4602 |
| ahi1 | NM_026203 | 52906 | 54806 |
| runx1 | NM_009821; | 12394 | 861 |
| | NM_001111021; | | |
| | NM_001111022; | | |
| | NM_001111023 | | |
| ets2 | NM_011809 | 23872 | 2114 |
| tmprss2 | NM_015775 | 50528 | 7113 |
| ripk4 | NM_023663 | 72388 | 54101 |
| erg | NM_133659 | 13876 | 2078 |
| gnb2 | NM_010312 | 14693 | 2783 |
| perq1 | NM_031408 | 57330 | 64599 |
| tox | NM_145711 | 252838 | 9760 |
| set | NM_023871 | 56086 | 6418 |
| fnbp1 | NM_001038700; | 14269 | 23048 |
| | NM_019406 | | |
| abl1 | NM_001112703; | 11350 | 25 |
| | NM_009594 | | |
| nup214 | NM_172268 | 227720 | 8021 |
| trp53 | NM_011640.3 | 22059 | 7157 |
| bcl6 | NM_009744 | 12053 | 604 |
| negr1 | NM_001039094; | 320840 | 257194 |
| | NM_177274 | | |
| baalc | NM_080640 | 118452 | 79870 |
| fzd6 | NM_008056 | 14368 | 8323 |
| crebbp | NM_001025432 | 12914 | 1387 |
| c2ta | NM_007575 | 12265 | 4261 |
| mxi1 | NM_010847; | 17859 | 4601 |
| | NM_001008542; | | |
| | NM_001008543 | | |
| hes3 | NM_008237 | 15207 | 390992 |
| rpl22 | NM_009079 | 19934 | 6146 |
| chd5 | NM_001081376 | 269610 | 26038 |
| ikaros | NM_009578 | 22778 | 10320 |
| ptprn2 | NM_011215 | 19276 | 5799 |
| tcrb | | 21577 | 6957 |
| gnaq | NM_008139 | 14682 | 2776 |
| pten | NM_008960 | 19211 | 5728 |
| fbxw7 | NM_080428 | 50754 | 55294 |

1. A non-human transgenic mammal that is genetically modified to develop cancer, such that the genome of a cancer cell from the mammal comprises chromosomal structural aberrations at a frequency that is at least 5-fold higher than the frequency of chromosomal structural aberrations in such mammal without the genetic modification.

2. The non-human transgenic mammal according to claim 1 which is a rodent.

3. The non-human transgenic mammal according to claim 1, which is a mouse.

4. The non-human transgenic mammal according to claim 1 that comprises engineered inactivation of
(a) at least one allele of one or more genes encoding a protein involved in DNA repair function and at least one allele of one or more genes encoding a component that synthesizes and maintains telomere length; or
(b) at least one allele of one or more genes encoding a protein involved in DNA repair function and at least one allele of one or more genes encoding a DNA damage checkpoint protein; or
(c) at least one allele of one or more genes encoding a DNA damage checkpoint protein and at least one allele of one or more genes encoding a component that synthesizes and maintains telomere length.

5. The non-human transgenic mammal according to claim 4, wherein the one or more genes encoding a protein involved in DNA repair function is selected from the group consisting of a protein involved in non-homologous end joining (NHEJ), a protein involved in homologous recombination, and a DNA repair helicase.

6. The non-human transgenic mammal according to claim 5, wherein the protein involved is NHEJ selected from the group consisting of Ligase4, XRCC4, H2AX, DNAPKcs, Ku70, Ku80, Artemis, Cernunnos/XLF, MRE11, NBS1, and RAD50.

7. The non-human transgenic mammal according to claim 5, wherein the protein involved in homologous recombination is selected from the group consisting of RAD51, RAD52, RAD54, XRCC3, RAD51C, BRCA1, BRCA2 (FANCD1), FANCA, FANCB, FANCC, FANCD2, FANCE, FANCF, FANCG, FANCJ (BRIP1/BACH1), FANCL, and FANCM.

8. The non-human transgenic mammal according to claim 5, wherein the DNA repair helicase is selected from the group consisting of BLM and WRN.

9. The non-human transgenic mammal according to claim 4, wherein the one or more genes encoding a DNA damage checkpoint protein is selected from the group consisting of p53, p21, APC, ATM, ATR, BRCA1, MDM2, MDM4, CHK1, CHK2, MRE11, NBS1, RAD50, MDC1, SMC1, ATRIP, and claspin.

10. The non-human transgenic mammal according to claim 4, wherein one or more genes encoding a component that synthesizes or maintains telomere length is a protein maintaining telomere structure.

11. The non-human transgenic mammal according to claim 10, wherein the protein maintaining telomere structure is selected from the group consisting of TRF1, TRF2, POT1a, POT1b, RAP 1, TIN2, and TPP1.

12. The non-human transgenic mammal according to claim 1, wherein the mammal is engineered for decreased telomerase activity.

13. The non-human transgenic mammal according to claim 4 or 12, wherein at least one allele of a telomerase reverse transcriptase (tert) gene is inactivated.

14. The non-human transgenic mammal according to claim 13, wherein both alleles of the telomerase reverse transcriptase (tert) gene are inactivated.

15. The non-human transgenic mammal according to claim 4 or 12, wherein at lease one allele of a telomerase RNA (terc) gene is inactivated.

16. The non-human transgenic mammal according to claim 15, wherein both alleles of the telomerase RNA (terc) gene are inactivated.

17. The non-human transgenic mammal according to any one of claims 1, 12 or 15, wherein at least one allele of p53 is inactivated.

18. The non-human transgenic mammal according to claim 17, wherein both alleles of p53 are inactivated.

19. The non-human transgenic mammal according to any one of claims 1, 12, 15 or 17, wherein at least one allele of the ataxia telangiectasia mutated (atm) gene is inactivated.

20. The non-human transgenic mammal according to any one of claims 1, 12, 15 or 17, wherein both alleles of the ataxia telangiectasia mutated (atm) gene are inactivated.

21. The non-human transgenic mammal according to claim 1, wherein the genome of the mammal comprises at least one additional cancer-promoting modification.

22. The non-human transgenic mammal according to claim 21, wherein the at least one additional cancer-promoting modification is an activated oncogene, an inactivated tumor suppressor gene, or both.

23. The non-human transgenic mammal according to claim 22, wherein the activated oncogene or the inactivated tumor suppressor gene is a recombinant gene.

24. The non-human transgenic mammal according to claim 21, wherein the additional cancer-producing modification is inducible.

25. The non-human transgenic mammal according to claim 21, wherein the additional cancer-producing modification is tissue-specific.

26. The non-human transgenic mammal according to claims 22, 24, or 25, wherein the additional cancer-producing modification is Kras activation.

27. The non-human transgenic mammal according to claim 26, wherein the activation of Kras is pancreas-specific.

28. A method of identifying a chromosomal region of interest for the identification of a gene or genetic element that is potentially related to human cancer, comprising the step of identifying a DNA copy number alteration in a population of cancer cells from a non-human mammal, wherein the genome of the non-human mammal is engineered to produce chromosomal instability, wherein the chromosomal region of the DNA copy number alteration is a chromosomal region of interest for the identification of a gene or genetic element that is potentially related to human cancer.

29-61. (canceled)

62. A method of identifying a chromosomal region of interest for the identification of a gene or genetic element that is potentially related to human cancer, comprising the step of identifying a chromosomal structural aberration in a population of cancer cells from a non-human mammal, wherein the genome of the non-human mammal is engineered to produce genome instability, wherein a chromosomal region containing the chromosomal structural aberration is a chromosomal region of interest for the identification of a gene or genetic element that is potentially related to human cancer.

63-67. (canceled)

68. A method for identifying a potential human cancer-related gene, comprising the steps of
(a) identifying a chromosomal region of interest by the method of claim 28 or 62;
(b) identifying a gene or genetic element within the chromosomal region of interest in the non-human mammal, and
(c) identifying a human gene or genetic element that corresponds to the gene or genetic element identified in step (b), wherein the human gene or genetic element is a potential human cancer-related gene or genetic element.

69-70. (canceled)

71. A method of identifying a potential human cancer-related gene or genetic element, comprising the steps of:
(a) detecting a DNA copy number alteration in a population of cancer cells from a non-human mammal, wherein the genome of the non-human mammal is engineered to produce genome instability,
(b) identifying a gene or genetic element located within the boundaries of the DNA copy number alteration detected in step (a),
(c) identifying a human gene or genetic element that corresponds to the gene or genetic element identified in step (b) and that is located within the boundaries of a DNA copy number alteration or of a chromosomal structural aberration in a human cancer cell;
wherein the human gene or genetic element identified in step (c) is a gene or genetic element potentially related to human cancer.

72. (canceled)

73. A method of identifying a potential human cancer-related gene or genetic element, comprising the steps of
(a) detecting a chromosomal structural aberration in a population of cancer cells from a non-human mammal, wherein the genome of the non-human mammal is engineered to produce genome instability,
(b) identifying a gene or genetic element located at the site of the chromosomal structural aberration detected in step (a),
(c) identifying a human gene or genetic element that corresponds to the gene or genetic element identified in step (b) and that is located within the boundaries of a DNA copy number alteration or at the site of a chromosomal structural aberration in a human cancer cell, wherein the human gene or genetic element identified in step (c) is a gene or genetic element potentially related to human cancer.

74-85. (canceled)

86. A method for identifying subjects with T-cell acute lymphoblastic leukemia (T-ALL) who may have a decreased response to γ-secretase inhibitor therapy, comprising: detecting the expression or activity of FBXW7 in a tumor cell from the subject, wherein a decreased expression or activity of FBXW7, as compared to a control, is indicative that the subject may have a decreased response to γ-secretase inhibitor therapy.

87-110. (canceled)

111. A method for identifying subjects with T-ALL that may benefit from treatment with a PI3K pathway inhibitor, comprising: detecting the expression or activity of PTEN in a tumor cell from the subject, wherein a decreased expression or activity of PTEN, as compared to a control, is indicative that the subject may benefit from a treatment with a PI3K inhibitor.

112-129. (canceled)

130. A method of assessing whether a subject is afflicted with cancer or at risk for developing cancer, comprising: determining the expression or activity level of at least one cancer gene or candidate cancer gene located in an amplified MCR in Table 1 in a biological sample from the subject; wherein an increase in the expression or activity the gene, as compared to a control, indicates that the subject is afflicted with cancer or at risk for developing cancer.

131. A method of assessing whether a subject is afflicted with cancer or at risk for developing cancer, comprising: determining the expression or activity level of at least one cancer gene or candidate cancer gene located in a deleted MCR in Table 1 in a biological sample from the subject; wherein a decrease in the expression or activity the gene, as compared to a control, indicates that the subject is afflicted with cancer or at risk for developing cancer.

132-133. (canceled)

134. A method of assessing whether a subject is afflicted with cancer or at risk for developing cancer, the method comprising: determining the copy number of at least one amplified minimal common region (MCR) listed in Table 1 in a biological sample from the subject; wherein an increased copy number of the MCR in the sample, as compared to the normal copy number of the MCR, indicates that the subject is afflicted with cancer or at risk for developing cancer.

135. A method of assessing whether a subject is afflicted with cancer or at risk for developing cancer, the method comprising: determining the copy number of at least one deleted minimal common region (MCR) listed in Table 1 in a biological sample from the subject; wherein a decreased copy number of the MCR in the sample, as compared to the normal copy number of the MCR, indicates that the subject is afflicted with cancer or at risk for developing cancer.

136-137. (canceled)

138. A method for monitoring the progression of cancer in a subject, the method comprising:
a) determining in a biological sample from the subject at a first point in time, the expression or activity level of a cancer gene or a candidate cancer gene listed in Table 1;
b) repeating step a) at a subsequent point in time; and
c) comparing the expression or activity of the gene in steps a) and b), and therefrom monitoring the progression of cancer in the subject.

139-140. (canceled)

141. A method of assessing the efficacy of a test agent for treating a cancer in a subject, the method comprising:
a) determining the expression or activity level of at least one cancer gene or a candidate cancer gene located in an amplified MCR in Table 1 in a biological sample from the subject in the presence of the test agent; and
b) determining the expression or activity level of the gene in a biological sample from the subject in the absence of the test agent, wherein a decreased expression or activity of the gene in step (a), as compared to that of (b), is indicative of the test agent's potential efficacy for treating the cancer in the subject.

142. A method of assessing the efficacy of a test agent for treating a cancer in a subject, the method comprising:
a) determining the expression or activity level of at least one cancer gene or a candidate cancer gene located in a deleted MCR in Table 1 in a biological sample from the subject in the presence of the test agent; and
b) determining the expression or activity level of the gene in a biological sample from the subject in the absence of the test agent, wherein an increased expression or activity of the gene in step (a), as compared to that of (b), is indicative of the test agent's potential efficacy for treating the cancer in the subject.

143-144. (canceled)

145. A method of assessing the efficacy of a therapy for treating cancer in a subject, the method comprising:
a) determining the expression or activity level of at least one cancer gene or a candidate cancer gene located in an amplified MCR in Table 1 in a biological sample from the subject prior to providing at least a portion of the therapy to the subject; and
b) determining the expression or activity level of the gene in a biological sample from the subject following provision of the portion of the therapy,
wherein a decreased expression or activity of the gene in step (a), as compared to that of (b), is indicative of the therapy's efficacy for treating the cancer in the subject.

146. A method of assessing the efficacy of a therapy for treating cancer in a subject, the method comprising:
a) determining the expression or activity level of at least one cancer gene or a candidate cancer gene located in a deleted MCR in Table 1 in a biological sample from the subject prior to providing at least a portion of the therapy to the subject; and
b) determining the expression or activity level of the gene in a biological sample from the subject following provision of the portion of the therapy,
wherein an increased expression or activity of the gene in step (a), as compared to that of (b), is indicative of the therapy's efficacy for treating the cancer in the subject.

147-148. (canceled)

149. A method of treating a subject afflicted with cancer comprising administering to the subject an agent that decreases the expression or activity level of at least one cancer gene or candidate cancer gene located in am amplified MCR in Table 1.

150. A method of treating a subject afflicted with cancer comprising administering to the subject an agent that increases the expression or activity level of at least one cancer gene or candidate cancer gene located in a deleted MCR in Table 1.

151-153. (canceled)

154. A method of assessing whether a subject is afflicted with cancer or at risk for developing cancer, the method comprising: determining the copy number of at least one minimal common region (MCR) listed in Table 5 in a biological sample from the subject; wherein a change of copy number of the MCR in the sample, as compared to the normal copy number of the MCR, indicates that the subject is afflicted with cancer or at risk for developing cancer.

155-156. (canceled)
